(19) United States
(12) Patent Application Publication
(10) Pub. No.: US 2026/0077004 A1
(43) Pub. Date: Mar. 19, 2026

(54) COMPOSITIONS AND METHODS FOR TREATMENT OF CARDIAC DISORDERS

(71) Applicant: University of Massachusetts, Westborough, MA (US)

(72) Inventors: Kevin Donahue, Worcester, MA (US); Phillip Tai, Worcester, MA (US); Guangping Gao, Worcester, MA (US)

(73) Assignee: University of Massachusetts, Westborough, MA (US)

(21) Appl. No.: 19/248,947

(22) Filed: Jun. 25, 2025

Related U.S. Application Data
(63) Continuation of application No. PCT/US2024/039783, filed on Jul. 26, 2024.
(60) Provisional application No. 63/515,856, filed on Jul. 27, 2023.

(51) Int. Cl. A61K 35/76; A61K 38/00; A61K 48/00; C07K 14/005; C07K 14/705; C12N 15/86

(52) U.S. Cl. CPC A61K 35/76; A61K 48/0058; C07K 14/005; C07K 14/705; C12N 15/86; A61K 38/00; C12N 2750/14143; C12N 2830/008

(57) ABSTRACT

Some aspects of the disclosure provide truncated potassium voltage-gated channel subfamily H member 2 (KCNH2) proteins. In some aspects, the disclosure provides nucleic acid regulatory elements comprising (i) an atrial natriuretic peptide (Anf) promoter and (ii) an enhancer element, wherein the enhancer element is not a naturally-occurring Anf enhancer element.

### RELATED APPLICATION

This application claims the benefit under 35 U.S.C. § 365 (c) and § 120 and is a continuation of International Patent Application Number PCT/US2024/039783, filed Jul. 26, 2024, titled “COMPOSITIONS AND METHODS FOR TREATMENT OF CARDIAC DISORDERS”, which claims priority under 35 U.S.C. § 119 (e) to U.S. provisional patent application 63/515,856, filed on Jul. 27, 2023, the entire content of each of which is incorporated by reference herein.

### REFERENCE TO AN ELECTRONIC SEQUENCE LISTING

The contents of the electronic sequence listing (U012070179US01-SEQ-KZM.xml; Size: 78,482 bytes; and Date of Creation: Jun. 24, 2025) is herein incorporated by reference in its entirety.

### FEDERALLY SPONSORED RESEARCH

This invention was made with government support under HL130376, HL134185 and HL158143 awarded by the National Institutes of Health. The government has certain rights in the invention.

### BACKGROUND

Atrial Fibrillation (AF) is the most commonly sustained cardiac arrhythmia. AF greatly affects quality of life and increases the risk of hospitalization, morbidity, and mortality for those afflicted with the condition. Genome wide association studies have identified KCNH2 as a genetic locus associated with AF, highlighting the potential of gene therapy as a method for treating AF in patients. However, the option of gene therapy in these patients is limited, as rAAV (the most promising and safest method of gene therapy available) is limited by the size of transgenes that an rAAV can carry. The packaging of AAV is limited to <5 kb, which excludes larger therapeutic genes from conventional vector designs.

### SUMMARY

Aspects of the disclosure relate to compositions and methods useful for treating atrial-restricted cardiac diseases. The disclosure is based, in part, on isolated nucleic acids (e.g., rAAV vectors) and rAAVs engineered to express transgenes encoding KCNH2 or variants thereof. In some embodiments, the transgenes encode a dominant-negative variant of KCNH2, such as KCNH2 comprising a G628S amino acid substitution. In some embodiments, the dominant-negative KCNH2 is truncated, allowing it to fit in an AAV vector. In some aspects, the disclosure is based on expression promoters that confer atrial-specific gene expression. In some embodiments, the promoters are hybrid promoters, comprising elements from multiple different regulatory elements (e.g., promoters and/or enhancer regions). In some embodiments, the atrial-specific promoters are packaged into rAAVs to confer atrial-specific transgene expression following rAAV transduction. In some embodiments, the transgenes encoding KCNH2 are under the control of the atrial-specific promoters disclosed herein. In some embodiments, a transgene encoding a truncated, functional G628S KCNH2 variant under the control of a hybrid atrial-specific promoter disclosed herein is packaged into an rAAV, which is used to treat atrial fibrillation.

Accordingly, in some aspects, provided herein are truncated potassium voltage-gated channel subfamily H member 2 (KCNH2) proteins comprising one or more dominant-negative mutations relative to a wild-type KCNH2 protein and having a length of 500 to 1,150 amino acids.

In some embodiments, the one or more dominant-negative KCNH2 mutations comprises one or more amino acid substitutions.

In some embodiments, the one or more amino acid substitutions is at position N470, T473, A561, I593, G626, F627, and/or G628 relative to amino acid position numbering of a wild-type KCNH2 protein (e.g., SEQ ID NO: 19).

In some embodiments, the amino acid substitution at position G628 is G628S, G628A, G628R, G628N, G628D, G628C, G628Q, G628E, G628H, G628I, G628L, G628K, G628M, G628F, G628P, G628S, G628T, G628W, G628Y, or G628V (e.g., SEQ ID NO: 22).

In some embodiments, the KCNH2 amino acid substitution at position N470 is N470D or N470E (e.g., SEQ ID NOs: 24 or 26).

In some embodiments, the amino acid substitution at position T473 is T473P (e.g., SEQ ID NO: 28).

In some embodiments, the amino acid substitution at position A561 is A561V (e.g., SEQ ID NO: 30).

In some embodiments, the amino acid substitution at position I593 is I593R or I593K (e.g., SEQ ID NOs: 32 or 34).

In some embodiments, the amino acid substitution at position G626 is G626S, G626A, G626R, G626N, G626D, G626C, G626Q, G626E, G626H, G626I, G626L, G626K, G626M, G626F, G626P, G626T, G626W, G626Y, or G626V.

In some embodiments, the amino acid substitution at position F627 is F627S, F627A, F627R, F627N, F627D, F627C, F627Q, F627E, F627H, F627I, F627L, F627K, F627M, F627G, F627P, F627T, F627W, F627Y, or F627V.

In some embodiments, the one or more dominant-negative KCNH2 mutations comprises a deletion.

In some embodiments, the deletion comprises a deletion of residues 500-508 relative to amino acid position numbering of a wild-type KCNH2 protein (e.g., SEQ ID NO: 35).

In some embodiments, the truncated KCNH2 protein is truncated by at least 10, 20, 30, 50, 100, 150, 200, 250, or more amino acids compared to the amino acid sequence set forth in SEQ ID NO: 19.

In some embodiments, the truncated KCNH2 protein consists of 500-1,000 amino acids, 500-900 amino acids, or 500-800 amino acids.

In some embodiments, the truncated KCNH2 protein consists of 650-950 amino acids. In some embodiments, the truncated KCNH2 protein consists of 910-930 amino acids.

In some embodiments, the truncated KCNH2 protein comprises at least 80%, 85%, 90%, 95%, 97%, 98%, or 99% identity to the amino acid sequence of SEQ ID NO: 21.

In some embodiments, the truncated KCNH2 protein comprises or consists of the amino acid sequence of SEQ ID NO: 22.

In some embodiments, the truncated KCNH2 protein suppresses delayed rectifier potassium current (I_{Kr}) channel function.

In some embodiments, provided herein are isolated nucleic acids encoding a truncated KCNH2 protein.

In some embodiments, the isolated nucleic acid comprises a nucleic acid sequence that is at least 70% identical to the nucleic acid sequence set forth in SEQ ID NOs: 1 or 2.

In some embodiments, the isolated nucleic acid comprises a nucleic acid sequence that is at least 60%, 70%, 80%, 90%, 95%, 99%, or 99.9% identical to the nucleic acid sequence set forth in SEQ ID NOs: 1 or 2.

In some embodiments, the isolated nucleic acid comprises a nucleic acid sequence that is 100% identical to the nucleic acid sequence set forth in SEQ ID NO: 2.

In some embodiments, the isolated nucleic acid further comprises a Kozak sequence.

In some embodiments, the isolated nucleic acid further comprises one or more regulatory elements.

In some embodiments, the one or more regulatory elements comprises a promoter. In some embodiments, a promoter is an atrial cell-specific promoter. In some embodiments, an atrial cell-specific promoter comprises an Anf1 promoter sequence (e.g., SEQ ID NO: 3).

In some embodiments, the one or more regulatory elements comprises an enhancer, optionally wherein the enhancer comprises a CMV enhancer sequence (SEQ ID NO: 4).

In some embodiments, the isolated nucleic acid further comprises one or more introns, optionally wherein the one or more introns is positioned between the one or more regulatory element and a nucleic acid sequence encoding the truncated KCNH2 protein.

In some embodiments, the isolated nucleic acid further comprises a 3′ untranslated region (3′ UTR).

In some embodiments, the isolated nucleic acid further comprises a 5′ untranslated region (5′ UTR).

In some embodiments, the isolated nucleic acid further comprises adeno-associated virus (AAV) inverted terminal repeats (ITRs) flanking a nucleotide sequence encoding the truncated KCNH2 protein.

In some embodiments, the AAV ITRs are of serotype AAV1, AAV2, AAV6, AAV8, or AAV9.

In some embodiments, provided herein are vectors comprising the isolated nucleic acids described herein.

In some embodiments, the vector is a plasmid or a viral vector.

In some embodiments, the viral vector is an adenoviral vector, adeno-associated viral vector, a lentiviral vector, a retroviral vector, or a Baculovirus vector.

In some embodiments, provided herein are host cells comprising the truncated KCNH2 proteins, the isolated nucleic acids, and/or the vectors described herein.

In some embodiments, the host cell is a mammalian cell, yeast cell, bacterial cell, or insect cell.

In some embodiments, provided herein are recombinant adeno-associated viruses (rAAVs) comprising:
(i) an isolated nucleic acid encoding the truncated KCNH2 protein described herein; and
(ii) an adeno-associated virus (AAV) capsid protein.

In some embodiments, the capsid protein has a tropism for heart tissue. In some embodiments, the heart tissue is atrial tissue.

In some embodiments, the capsid protein is of a serotype selected from AAV1, AAV2, AAV3, AAV4, AAV5, AAV6, AAV7, AAV8, AAV9, and a variant of any of the foregoing.

In some embodiments, the capsid protein is of serotype AAV1.

In some embodiments, the rAAV is formulated for delivery to heart tissue of a subject.

In some embodiments, provided herein are compositions comprising the truncated KCNH2 proteins, the isolated nucleic acids, the vectors, and/or the rAAVs described herein.

In some embodiments, the composition further comprises a pharmaceutically acceptable excipient.

In some embodiments, provided herein are methods of administering a truncated KCNH2 protein to a subject, the method comprising administering to the subject the isolated nucleic acids, the rAAVs, and/or the compositions described herein.

In some embodiments, provided herein are methods of decreasing activity of wild-type KCNH2 in a subject, the method comprising administering to the subject the isolated nucleic acids, the rAAVs, and/or compositions described herein.

In some embodiments, the subject has a cardiac disorder.

In some embodiments, the subject has atrial fibrillation.

In some embodiments, provided herein are methods of treating atrial fibrillation in a subject in need thereof, comprising administering to the subject the compositions described herein.

In some embodiments, the subject is human.

In some embodiments, the administering is via gene painting.

In some embodiments, the administration is via injection, optionally intravenous injection.

In some embodiments, provided herein are methods of decreasing activity of wild-type KCNH2 in a cell, comprising administering to the cell the truncated KCNH2 proteins, the isolated nucleic acids, the vectors, the rAAVs, and/or the compositions described herein.

In some embodiments, the cell is cultured in vitro.

In some embodiments, provided herein are nucleic acid regulatory elements comprising (i) an atrial natriuretic peptide (Anf) promoter and (ii) an enhancer element, wherein the enhancer element is not a naturally occurring Anf enhancer element.

In some embodiments, the naturally occurring Anf enhancer element consists of the nucleic acid sequence set forth in SEQ ID NO: 6.

In some embodiments, the Anf promoter is a mouse Anf promoter or a human Anf promoter.

In some embodiments, the mouse Anf promoter comprises a nucleic acid sequence that is at least 70%, 80%, 90%, 95%, 97%, 98%, or 99% identical to the nucleic acid sequence set forth in SEQ ID NO: 3.

In some embodiments, the mouse Anf promoter comprises or consists of the nucleic acid sequence set forth in SEQ ID NO: 3.

In some embodiments, the human Anf promoter comprises a nucleic acid sequence that is at least 70%, 80%, 90%, 95%, 97%, 98%, or 99% identical to the nucleic acid sequence set forth in SEQ ID NO: 8.

In some embodiments, the human Anf promoter comprises or consists of the nucleic acid sequence as set forth in SEQ ID NO: 8.

In some embodiments, the enhancer element is a CMV enhancer.

In some embodiments, the CMV enhancer comprises a nucleic acid sequence that is at least 70%, 80%, 90%, 95%, 97%, 98%, or 99% identical to the nucleic acid sequence set forth in SEQ ID NO: 4.

In some embodiments, the CMV enhancer comprises or consists of the nucleic acid sequence as set forth in SEQ ID NO: 4.

In some embodiments, nucleic acid regulatory elements described herein comprise a nucleic acid that is at least 70%, 80%, 90%, 95%, 97%, 98%, or 99% identical to the nucleic acid sequence set forth in SEQ ID NOs: 5 or 9.

In some embodiments, the nucleic acid regulatory element comprises or consists of the nucleic acid sequence as set forth in SEQ ID NOs: 5 or 9.

In some embodiments, the nucleic acid regulatory elements described herein are operably linked to a nucleic acid sequence encoding a protein.

In some embodiments, the protein is a protein associated with one or more atrial-restricted cardiac diseases, optionally wherein the protein is a protein associated with atrial fibrillation.

In some embodiments, the protein is a voltage-gated channel protein.

In some embodiments, the voltage-gated protein is a potassium voltage-gated channel subfamily H member 2 protein (KCNH2 protein).

In some embodiments, the KCNH2 protein comprises an amino acid sequence that is at least 70%, 80%, 90%, 95%, 97%, 98%, or 99% identical to the amino acid sequence set forth in SEQ ID NO: 19 or 20.

In some embodiments, the KCNH2 protein comprises or consists of the nucleic acid sequence as set forth in SEQ ID NO: 19 or 20.

In some embodiments, the nucleic acid sequence encoding a protein is at least 60%, 70%, 80%, 90%, 95% or 99% identical to the sequence set forth in SEQ ID NOs: 1 or 2.

In some embodiments, the nucleic acid sequence encoding a protein comprises or consists of the nucleic acid sequence as set forth in SEQ ID NO: 1 or 2.

In some embodiments, the protein associated with one or more atrial-restricted cardiac diseases is Calcium/Calmodulin Dependent Protein Kinase II Inhibitor 2 (CAMK2N2), Sarcoplasmic/Endoplasmic Reticulum Calcium ATPase 2A (SERCA2A), Potassium Two-Pore Domain Channel Subfamily K Member 2 (KCNK2), Connexin 40, or Connexin 43.

In some embodiments, isolated nucleic acids described herein further comprise adeno-associated virus (AAV) inverted terminal repeats (ITRs).

In some embodiments, the AAV ITRs are of serotype AAV1, AAV2, AAV6, AAV8, or AAV9.

In some embodiments, provided herein are vectors comprising the isolated nucleic acids described herein.

In some embodiments, the vector is a plasmid or a viral vector.

In some embodiments, the viral vector is an adenoviral vector, adeno-associated viral vector, a lentiviral vector, a retroviral vector, or a Baculovirus vector.

In some embodiments, provided herein are recombinant adeno-associated viruses (rAAVs) comprising:
(i) the isolated nucleic acids described herein; and
(ii) an adeno-associated virus (AAV) capsid protein.

In some embodiments, the capsid protein has a tropism for heart tissue, optionally wherein the heart tissue is atrial tissue.

In some embodiments, the capsid protein is of a serotype selected from AAV1, AAV2, AAV3, AAV4, AAV5, AAV6, AAV7, AAV8, AAV9 and a variant of any of the foregoing.

In some embodiments, the rAAV is formulated for delivery to the heart.

In some embodiments, provided herein are compositions comprising the isolated nucleic acids, the vectors, and/or the rAAVs described herein.

In some embodiments, the compositions further comprise a pharmaceutically acceptable excipient.

In some embodiments, provided herein are methods for expressing a protein in an atrial cardiomyocyte comprising administering the isolated nucleic acids described herein or the compositions described herein to the atrial cardiomyocyte.

In some embodiments, the expression of the protein is increased by at least 10%, 20%, 30%, 50%, 75%, or 100% relative to a control cardiomyocyte.

In some embodiments, provided herein are methods for expressing a protein in an atrial cardiomyocyte of a subject comprising administering the isolated nucleic acids or the compositions described herein to the subject.

In some embodiments, the expression of the protein is higher in the atrial cardiomyocyte relative to a control cell in the subject, optionally wherein the control cell is a ventricular cardiomyocyte or a liver cell from the same subject.

In some embodiments, the expression of the protein is increased by at least 10%, 20%, 30%, 50%, 75%, or 100% in the atrial cardiomyocyte relative to the control cell.

In some embodiments, provided herein are methods of treating an atrial-restricted cardiac disease in a subject in need thereof, comprising administering to the subject the compositions described herein. In some embodiments, the atrial-restricted cardiac disease is atrial fibrillation.

In some embodiments, the subject has a cardiac disorder. In some embodiments, the subject has atrial fibrillation. In some embodiments, the subject is human.

In some embodiments, the administration is via gene painting. In some embodiments, the administration is intravenous, intramuscular, subcutaneous, or intraarterial administration.

### BRIEF DESCRIPTION OF DRAWINGS

FIG. 1 demonstrates the dominant-negative activity of truncated KCNH2 protein. The delayed rectifier potassium current (I_{Kr}) current measured in CHO cells expressing wild-type KCNH2 and transfected with vectors encoding GFP (control, left), full-length G628S KCNH2 protein (middle), or truncated G628S KCNH2 protein (right) showed a loss of tail current and sustained current with the addition of either full-length or truncated G628S KCNH2 protein.

FIG. 2 depicts the identification and sequence of a 1.58-kb atrial-specific Anf regulatory element. FIG. 2 shows the ENCODE gene regulation tracks of DNase-hypersensitivity (DHS) and histone modifications at the mouse Anf gene, revealing two distinct regulatory regions (enhancer region and promoter) in heart tissue. H3K4 m1, H3K4m3, H3K9a, H3K27a, H3K27m3 and H3K36m3 denote specific histone modifications and their locations.

FIGS. 3A-3B demonstrate the activity of the 1.58-kb Anf regulatory element compared to a CMV enhancer/chicken beta-actin ubiquitous promoter (CB6) in ventricular cardiomyocytes (H9C2) and atrial cardiomyocytes (HL-1). FIG. 3A shows the relative light units (RLU) detected from cells expressing firefly luciferase (FLuc) under the control of a CB6 ubiquitous promoter (CB6-FLuc; left) or 1.58-kb Anf regulatory element (ANF-FLuc; right). FIG. 3B shows the RLU from cells expressing the ANF-Luc as a percentage of the RLU from cells expressing the CB6-Luc in the same cell type, indicating the atrial specificity of the 1.58-kb Anf regulatory element.

FIG. 4 depicts schematics showing the different regions that make up the regulatory elements tested in the present application. Construct 1 (CMV-e/CMV-p) contains both the CMV enhancer and promoter regions. Construct 2 (0.97-kb Anf promoter) contains the Anf promoter identified through ENCODE and ChIP-Seq analyses (see the full 1.58-kb Anf regulatory element in FIGS. 2 and 3). Construct 3 contains a hybrid regulatory element with a CMV enhancer region and the Anf promoter region (CMV-e/Anf-p). Construct 4 contains a hybrid regulatory element with an Anf enhancer region and a CMV promoter region (Anf-e/CMV-p).

FIGS. 5A-5B demonstrate the enhanced activity and specificity of the engineered hybrid regulatory elements in comparison to the naturally occurring CMV and Anf promoters. FIG. 5A shows the relative light units (RLU) detected from cells expressing constructs 1-4 from FIG. 4 in H9C2 and HL-1 cells. FIG. 5B shows the RLU detected from rabbit hearts expressing FLuc driven by CB6, CMV-e/CMV-p, hybrid Anf-e/CMV-p and hybrid CMV-e/Anf-p regulatory elements.

### DETAILED DESCRIPTION

Aspects of the disclosure provide compositions and methods for treatment of diseases, disorders and conditions associated with the heart (e.g., cardiac disorders such as Atrial Fibrillation (AF)). The disclosure is based, in part, on compositions and methods for improved delivery of potassium voltage-gated channel subfamily H member 2 protein (KCNH2 protein) through the use of truncated KCNH2 constructs; and compositions and methods for improved nucleic acid delivery to the heart using synthetic atrial-specific regulatory elements. In some embodiments, isolated nucleic acids described herein encode a truncated dominant-negative form of KCNH2 (e.g., G628S KCNH2). In some embodiments, the isolated nucleic acids (e.g., transgenes or rAAV vectors) comprise engineered regulatory elements that confer high levels of atrial-specific gene expression.

### Potassium Voltage-Gated Channel Subfamily H Member 2 (KCNH2)

The disclosure relates, in some aspects, to truncated dominant-negative KCNH2 proteins. KCNH2 is a voltage-gated potassium channel encoded by the KCNH2 gene in humans (RefSeqGene NG_008916.1), which is expressed in cardiac muscle, nerve cells, and microglia, and plays a key role in cardiac myocyte repolarization. In some embodiments, wild-type KCNH2 protein comprises the amino acid sequence set forth in SEQ ID NO: 19. Without wishing to be bound by any particular theory, KCNH2 (also known as hERG) has been characterized as the pore-forming subunit of the rapid component of the delayed rectifier K⁺ current, making it an important component in controlling cardiac rhythm. A mutant variant of KCHN2, G628S, has been shown to have a dominant-negative effect on KCNH2 current, making it useful as a method to control cardiac rhythm. Other dominant-negative mutant variants of KCNH2 contemplated herein include substitution mutations at amino acid positions N470, A561, I593, G626, and/or F627 relative to wild-type KCNH2 (e.g., SEQ ID NO: 19). In some embodiments, a dominant-negative mutant variant of KCNH2 has a deletion of residues I500-F508 relative to wild-type KCNH2 (e.g., SEQ ID NO: 19)

Full-length wild-type KCNH2 (e.g., SEQ ID NO: 19) and G628S KCNH2 (e.g., SEQ ID NO: 20) are encoded by transcripts that are 3480 nucleotides in length (e.g., SEQ ID NO: 12-wild-type KCNH2, and SEQ ID NO: 13-G628S KCNH2). When combined with regulatory elements, the length of this gene strains the packaging capability of AAV vectors. The inventors realized that delivery of either the wild-type or G628S mutant using a recombinant AAV would be difficult, if not impossible at large scale. Aspects of the disclosure relate to truncated variants of G628S KCNH2 that maintain the dominant-negative functionality of the G628S KCNH2 mutant while reducing the total size of the G628S KCNH2 gene and protein, thereby enabling its delivery via recombinant AAV vectors.

In some embodiments, the disclosure relates to truncated dominant-negative mutant KCNH2 proteins (e.g., G628S KCNH2). In some embodiments, the truncated KCNH2 protein comprises (i) one or more amino acid substitutions that provide a dominant-negative phenotype (e.g., an amino acid substitution at position G628 relative to amino acid position numbering of a wild-type KCNH2 protein); and (ii) a truncation at a terminal end relative to a wild-type KCNH2 protein (e.g., a wild-type KCNH2 protein comprising the amino acid sequence of SEQ ID NO: 19). In some embodiments, the one or more amino acid substitutions that provide a dominant-negative phenotype is an amino acid substitution at any amino acid position if said substitution provides the dominant-negative phenotype (e.g., the dominant-negative phenotype described for G628S KCNH2 full-length mutant). As used herein, a “dominant-negative” mutation is an amino acid substitution or deletion that adversely affects the product of the wild-type protein or gene (e.g., KCNH2). For example, in some embodiments, a dominant-negative KCNH2 protein decreases the activity of wild-type KCNH2.

In some embodiments, the truncated KCNH2 protein comprises an amino acid substitution at position G628 (relative to amino acid positioning of a wild-type KCNH2 protein such as the KCNH2 protein having the amino acid sequence of SEQ ID NO: 19). In some embodiments, the truncated KCNH2 protein comprises an amino acid substitution at position G628 and at least one additional position. In some embodiments, the truncated KCNH2 protein comprises (a) an amino acid substitution at position G628; and (b) 1, 2, 3, 4, 5, 6, 7, 8, 9, or 10 additional amino acid substitutions relative to a wild-type KCNH2 protein.

In some embodiments, the amino acid substitution at position G628 introduces an amino acid having a polar side chain at position G628 (numbering relative to a wild-type KCNH2 protein having the amino acid sequence of SEQ ID NO: 19). In some embodiments, the amino acid substitution at position G628 introduces a serine, tyrosine, threonine, asparagine, or glutamine at position G628. In some embodiments, the truncated KCNH2 protein comprises a G628S, G628Y, G628T. G628N, or G628Q substitution.

In some embodiments, the truncated KCNH2 protein comprises a truncation at the N-terminal end relative to a wild-type KCNH2 protein (e.g., a KCNH2 protein having the amino acid sequence of SEQ ID NO: 19). In some embodiments, the truncated KCNH2 protein comprises a truncation at the C-terminal end relative to a wild-type KCNH2 protein (e.g., a KCNH2 protein having the amino acid sequence of SEQ ID NO: 19). In some embodiments, the truncated KCNH2 protein comprises a truncation at the N-terminal end relative to a wild-type KCNH2 protein (e.g., a KCNH2 protein having the amino acid sequence of SEQ ID NO: 19) and a truncation at the C-terminal end relative to a wild-type KCNH2 protein (e.g., a KCNH2 protein having the amino acid sequence of SEQ ID NO: 19).

In some embodiments, a wild-type KCNH2 protein having the sequence of SEQ ID NO: 19 consists of 1,159 amino acids. Thus, in some embodiments, a truncated variant of the wild-type KCNH2 protein having the sequence of SEQ ID NO: 19 consists of 1,158 amino acids or fewer. In some embodiments, the truncated KCNH2 protein consists of 500-1,158 amino acids, 500-1,000 amino acids, 500-900 amino acids, 500-800 amino acids, 500-700 amino acids, 500-600 amino acids, 600-1,000 amino acids, 600-900 amino acids, 650-1,000 amino acids, 650-950 amino acids, 700-950 amino acids, 800-950 amino acids, 900-950 amino acids, 910-950 amino acids, 910-930 amino acids, or 915-925 amino acids. In some embodiments, a truncated KCNH2 protein consists of 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, or 940 amino acids.

In some embodiments, the truncated KCNH2 protein comprises a truncation of 100-300 amino acids at the N-terminal end of a wild-type KCNH2 protein (e.g., having the amino acid sequence of SEQ ID NO: 19). In some embodiments, the truncated KCNH2 protein comprises a truncation of 200-300 amino acids, 200-250 amino acids, 250-300 amino acids, 220-260 amino acids, or 230-250 amino acids at the N-terminal end of a wild-type KCNH2 protein (e.g., having the amino acid sequence of SEQ ID NO: 19). In some embodiments, the truncated KCNH2 protein comprises a truncation of 100-300 amino acids at the C-terminal end of a wild-type KCNH2 protein (e.g., having the amino acid sequence of SEQ ID NO: 19). In some embodiments, the truncated KCNH2 protein comprises a truncation of 200-300 amino acids, 200-250 amino acids, 250-300 amino acids, 220-260 amino acids, or 230-250 amino acids at the C-terminal end of a wild-type KCNH2 protein (e.g., having the amino acid sequence of SEQ ID NO: 19). In some embodiments, the truncated KCNH2 protein comprises a truncation of 100-300 amino acids at the N-terminal end and a truncation of 100-300 amino acids at the C-terminal end of a wild-type KCNH2 protein (e.g., having the amino acid sequence of SEQ ID NO: 19). In some embodiments, the truncated KCNH2 protein comprises a truncation of 200-300 amino acids, 200-250 amino acids, 250-300 amino acids, 220-260 amino acids, or 230-250 amino acids at the N-terminal end and a truncation of 200-300 amino acids, 200-250 amino acids, 250-300 amino acids, 220-260 amino acids, or 230-250 amino acids at the C-terminal end of a wild-type KCNH2 protein (e.g., having the amino acid sequence of SEQ ID NO: 19).

In some embodiments, the truncated KCNH2 protein comprises a truncation of 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids at the N-terminal end of a wild-type KCNH2 protein (e.g., having the amino acid sequence of SEQ ID NO: 19). In some embodiments, the truncated KCNH2 protein comprises a truncation of 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids at the C-terminal end of a wild-type KCNH2 protein (e.g., having the amino acid sequence of SEQ ID NO: 19). In some embodiments, the truncated KCNH2 protein comprises a truncation of 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids at the C-terminal end and a truncation of 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids at the N-terminal end of a wild-type KCNH2 protein (e.g., having the amino acid sequence of SEQ ID NO: 19). In some embodiments, the truncated KCNH2 protein is post-translationally processed in the same way as a wild-type KCNH2 protein (e.g., having the amino acid sequence of SEQ ID NO: 19). In some embodiments, the truncated KCNH2 protein is at least 90%, at least 95%, at least 96%, at least 97% at least 98%, at least 99%, at least 99.5%, or is 100% identical to a C-terminal region of a wild-type KCNH2 protein (e.g., having the amino acid sequence of SEQ ID NO: 19). In some embodiments, a C-terminal region of a wild-type KCNH2 protein encompasses any amino acid beyond amino acid position 660 relative to amino acid position numbering of the wild-type KCNH2 protein. In some embodiments, the truncated KCNH2 protein comprises a C-terminal cyclic nucleotide binding (cNBD) domain. In some embodiments, the cNBD domain comprises the amino acids belonging to amino acid positions 742-824 relative to amino acid numbering of a wild-type KCNH2 protein. In some embodiments, the truncated KCNH2 protein comprises one or more C-terminal RXR motifs. In some embodiments, the one or more RXR motifs in the truncated KCNH2 protein are RGR motifs. In some embodiments, the RGR motifs in the truncated KCNH2 protein are at amino acid positions 920-922 and 1005-1007 relative to amino acid numbering of a wild-type KCNH2 protein (e.g., having the amino acid sequence of SEQ ID NO: 19).

In some embodiments, a truncated dominant-negative mutant KCNH2 protein comprises at least 80%, 85%, 90%, 95%, 97%, 98%, or 99% identity to the amino acid sequence of SEQ ID NO: 21.

In some embodiments, a truncated dominant-negative KCNH2 protein contains a substitution mutation at amino acid position G628 with reference to a truncated wild-type KCNH2 protein (e.g., having an amino acid sequence of SEQ ID NO: 21). Without wishing to be bound by any theory, the dominant-negative effect of the G628S KCNH2 mutation is caused by size hindrance introduced by the substitution of glycine with an amino acid containing a larger side chain (e.g., serine). Thus, in some embodiments, substitution of G628 with any amino acid containing a side chain larger than a single hydrogen (i.e., any amino acid besides glycine) will confer a dominant-negative effect.

In some embodiments, a truncated dominant-negative mutant KCNH2 protein (e.g., G628S KCNH2) comprises or consists of the amino acid sequence set forth in SEQ ID NO: 22. In some embodiments, a truncated dominant-negative mutant KCNH2 protein (e.g., G628S KCNH2) comprises at least 70%, 80%, 90%, 95%, or 99% identical to the amino acid sequence set forth in SEQ ID NO: 22. In some embodiments, a G628S KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 22.

In some embodiments, a truncated dominant-negative mutant KCNH2 protein (e.g., N470D KCNH2) comprises or consists of the amino acid sequence set forth in SEQ ID NO: 24. In some embodiments, a truncated dominant-negative mutant KCNH2 protein (e.g., N470D KCNH2) comprises at least 70%, 80%, 90%, 95%, or 99% identical to the amino acid sequence set forth in SEQ ID NO: 24. In some embodiments, a N470D KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 24.

In some embodiments, a truncated dominant-negative mutant KCNH2 protein (e.g., N470E KCNH2) comprises or consists of the amino acid sequence set forth in SEQ ID NO: 26. In some embodiments, a truncated dominant-negative mutant KCNH2 protein (e.g., N470E KCNH2) comprises at least 70%, 80%, 90%, 95%, or 99% identical to the amino acid sequence set forth in SEQ ID NO: 26. In some embodiments, a N470E KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 26.

In some embodiments, a truncated dominant-negative mutant KCNH2 protein (e.g., T473P KCNH2) comprises or consists of the amino acid sequence set forth in SEQ ID NO: 28. In some embodiments, a truncated dominant-negative mutant KCNH2 protein (e.g., T473P KCNH2) comprises at least 70%, 80%, 90%, 95%, or 99% identical to the amino acid sequence set forth in SEQ ID NO: 28. In some embodiments, a T473P KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 28.

In some embodiments, a truncated dominant-negative mutant KCNH2 protein (e.g., A561V KCNH2) comprises or consists of the amino acid sequence set forth in SEQ ID NO: 30. In some embodiments, a truncated dominant-negative mutant KCNH2 protein (e.g., A561V KCNH2) comprises at least 70%, 80%, 90%, 95%, or 99% identical to the amino acid sequence set forth in SEQ ID NO: 30. In some embodiments, an A561V KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 30.

In some embodiments, a truncated dominant-negative mutant KCNH2 protein (e.g., I593R KCNH2) comprises or consists of the amino acid sequence set forth in SEQ ID NO: 32. In some embodiments, a truncated dominant-negative mutant KCNH2 protein (e.g., I593R KCNH2) comprises at least 70%, 80%, 90%, 95%, or 99% identical to the amino acid sequence set forth in SEQ ID NO: 32. In some embodiments, an I593R KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 32.

In some embodiments, a truncated dominant-negative mutant KCNH2 protein (e.g., I593K KCNH2) comprises or consists of the amino acid sequence set forth in SEQ ID NO: 34. In some embodiments, a truncated dominant-negative mutant KCNH2 protein (e.g., I593K KCNH2) comprises at least 70%, 80%, 90%, 95%, or 99% identical to the amino acid sequence set forth in SEQ ID NO: 34. In some embodiments, an I593K KCNH2 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 34.

In some embodiments, a truncated dominant-negative mutant KCNH2 protein (e.g., KCNH2 4500-508) comprises or consists of the amino acid sequence set forth in SEQ ID NO: 35. In some embodiments, a truncated dominant-negative mutant KCNH2 protein (e.g., KCNH2 Δ500-508) comprises at least 70%, 80%, 90%, 95%, or 99% identical to the amino acid sequence set forth in SEQ ID NO: 35. In some embodiments, a KCNH2 Δ500-508 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NO: 35.

“Homology” refers to the percent identity between two polynucleotides or two polypeptide moieties. The term “substantial homology”, when referring to a nucleic acid or fragment thereof, indicates that, when optimally aligned with appropriate nucleic acid insertions or deletions with another nucleic acid (or its complementary strand), there is nucleotide sequence identity in about 90% to 100% of the aligned sequences. When referring to a polypeptide, or fragment thereof, the term “substantial homology” indicates that, when optimally aligned with appropriate gaps, insertions, or deletions with another polypeptide, there is amino acid identity in about 90% to 100% of the aligned sequences. The term “highly conserved” means at least 80% identity, preferably at least 90% identity, and more preferably over 97% identity. In some cases, “highly conserved” may refer to 100% identity. Identity is readily determined by one of skill in the art by, for example, the use of algorithms and computer programs known by those of skill in the art.

As used herein, alignments between sequences of nucleic acids or polypeptides are performed using any of a variety of publicly or commercially available Multiple Sequence Alignment programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences. e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. In some embodiments, alignments are used to identify corresponding amino acids between two peptides or proteins. A “corresponding amino acid” is an amino acid of a protein or peptide sequence that has been aligned with an amino acid of another protein or peptide sequence. In some embodiments, corresponding amino acids are identical. In some embodiments, corresponding amino acids are non-identical. In some embodiments, a corresponding amino acid that is a non-identical amino acid is referred to as a variant amino acid.

Alternatively, for nucleic acids homology can be determined by hybridization of polynucleotides under conditions that form stable duplexes between homologous regions, followed by digestion with single-stranded-specific nuclease(s), and size determination of the digested fragments. DNA sequences that are substantially homologous can be identified, for example, in a Southern hybridization experiment under conditions as defined for that particular system.

Mutations contemplated herein, with respect to an amino acid sequence, include, without limitation, substitutions, deletions, and additions. An amino acid “substitution” is a change in a single amino acid relative to a reference amino acid sequence. An amino acid substitution may result in a change in charge of the side chain of the amino acid position (e.g., from negatively charged to positively charged). In some embodiments, an amino acid substitution results in a change in polarity or hydrophobicity of the side chain of the amino acid position. In some embodiments, an amino acid substitution is a conservative substitution (e.g., a change from valine to alanine). In some embodiments, an amino acid substitution results in a different amino acid at that position that has an “equivalent” charge, polarity, and or chemical class (defined by the amino acid side chain).

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. In some embodiments, an isolated nucleic acid is substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that in some embodiments, conservative amino acid substitutions are made to provide functionally equivalent variants, or homologs of the truncated proteins described herein. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

### Atrial-Specific Regulatory Elements

Aspects of the disclosure relate to novel atrial-specific regulatory elements; and nucleic acids and vectors, for example viral vectors, that comprise said atrial-specific regulatory elements. In some embodiments, hybrid atrial-specific regulatory elements comprise (i) an atrial natriuretic peptide (Anf) promoter and (ii) an enhancer element, wherein the enhancer element is not a naturally-occurring Anf enhancer element. In some embodiments, such hybrid promoters are effective in driving expression of transgenes within cardiac tissues (e.g., the atrium of the heart).

In some embodiments, the Anf promoter is derived from an Anf promoter belonging to any species. In some embodiments, the Anf promoter is an animal Anf promoter (e.g., a mammalian Anf promoter or an amphibian Anf promoter). For example, in some embodiments, the Anf promoter is a human, mouse, rat, monkey, dog, cat, horse, cow, or Xenopus Anf promoter. In some embodiments, the Anf promoter is a mouse Anf promoter. In other embodiments, the Anf promoter is a human Anf promoter. In some embodiments, the Anf promoter is as described in Nemer, M. et al., The atrial natriuretic factor promoter is a downstream target for Nkx-2.5 in the myocardium. Mol Cell Biol. 1996 September; 16 (9): 4648-4655.; or Small, E. M. and Krieg, P. A., Transgenic analysis of the atrial natriuretic factor (ANF) promoter: Nkx2-5 and GATA-4 binding sites are required for atrial specific expression of ANF. Dev Biol. 2003 Sep. 1; 261 (1): 116-31, the contents of each of which are incorporated herein by reference.

In some embodiments, the Anf promoter comprises or consists of the nucleic acid sequence set forth in SEQ ID NO: 3. In some embodiments, the Anf promoter comprises a nucleic acid sequence that is at least 60%, at least 70%, 80%, 90%, 95%, 97%, 98%, or 99% identical to the nucleic acid sequence set forth in SEQ ID NO: 3. In some embodiments, the Anf promoter comprises or consists of the nucleic acid sequence set forth in SEQ ID NO: 8. In some embodiments, the Anf promoter comprises a nucleic acid sequence that is at least 60%, at least 70%, 80%, 90%, 95%, 97%, 98%, or 99% identical to the nucleic acid sequence set forth in SEQ ID NO: 8.

In some embodiments, the hybrid atrial-specific regulatory elements comprise one or more enhancer elements that are not naturally occurring Anf enhancer elements. In some embodiments, the hybrid atrial-specific regulatory elements comprise one or more enhancer elements that are not naturally linked to an Anf promoter or Anf gene. Thus, in some embodiments, the enhancer element of the hybrid regulatory element is any other enhancer element that is known to a person of ordinary skill in the art (that is not naturally linked to an Anf promoter or Anf gene). Such enhancers are typically short (50-1500 base pairs) regions of DNA that can be bound by transcriptional proteins (e.g., transcriptional activator proteins) to increase the likelihood that transcription of the downstream transgene will occur. As used herein, the term “enhancer element” refers to a nucleic acid sequence that when bound by an activator protein, activates or increases transcription of a gene or genes. Enhancer sequences can be upstream (i.e., 5′) or downstream (i.e., 3′) relative to the genes they regulate. In some embodiments, an enhancer is as described in Bejerano, G. et. al., Enhancers: five essential questions., Nature Reviews Genetics volume 14, pages 288-295 (2013), the contents of which are incorporated herein by reference. Examples of enhancer sequences include cytomegalovirus (CMV) enhancer sequence and the SV40 enhancer sequence.

In some embodiments, the enhancer element is a cytomegalovirus (CMV) enhancer, chicken beta-actin (CBA) enhancer, a retroviral Rous sarcoma virus (RSV) enhancer (e.g., an RSV long terminal repeat (LTR) enhancer), a Simian vacuolating virus 40 (SV40) enhancer, a dihydrofolate reductase enhancer, a beta-actin enhancer, a phosphoglycerol kinase (PGK) enhancer, or an EF1alpha enhancer. In some embodiments, the CMV enhancer comprises a nucleic acid sequence that is at least 70%, 80%, 90%, 95%, 97%, 98%, or 99% identical to the nucleic acid sequence set forth in SEQ ID NO: 4. In some embodiments, the CMV enhancer comprises or consists of the nucleic acid sequence as set forth in SEQ ID NO: 4.

In some embodiments, a nucleic acid sequence encoding a gene product is operably linked to an atrial-specific regulatory element (e.g., as described in SEQ ID NOs: 5 or 9). In some embodiments, a hybrid regulatory element comprises a mammalian Anf1 promoter, or variant thereof (e.g., as described in SEQ ID NOs: 3 and 8). In some embodiments, the hybrid regulatory element comprises a CMV enhancer, or variant thereof (e.g., as described in SEQ ID NO: 4). In some embodiments, a hybrid regulatory element variant comprises a nucleic acid sequence that has at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with the nucleic acid sequence set forth in one of SEQ ID NOs: 5 or 9. In some embodiments, a hybrid regulatory element comprises a promoter comprising a nucleic acid sequence that has at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with the nucleic acid sequence set forth in one of SEQ ID NOs: 3 or 8. In some embodiments, a hybrid regulatory element comprises an enhancer comprising a nucleic acid sequence that has at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with the nucleic acid sequence set forth in one of SEQ ID NO: 4. In some embodiments, a hybrid regulatory element is preferred when tissue-specific expression is desired with high, non-native levels of transgene expression (e.g., atrial cell-specific expression above native expression levels). Without wishing to be bound by any theory, use of a hybrid regulatory element comprising a CMV enhancer/Anf1 promoter (CMV-e/Anf1-p) in isolated nucleic acids and rAAV vectors described herein regulates expression of gene products from the vectors, and reduces toxicity, for example cytotoxicity or hepatotoxicity, in a subject relative to expression of the gene products from isolated nucleic acids and rAAV vectors comprising other regulatory elements, for example CMV promoter, chicken-beta actin (CBA) promoter, CB6 promoter, etc. In a further embodiment, other expression control elements, such as polyadenylation sites and/or Kozak consensus sequences are used to mimic the expression of a transgene.

In some embodiments, a mammalian Anf1 promoter preferentially drives expression of a gene product encoded by a nucleic acid in certain tissues. In some embodiments, a mammalian Anf1 promoter preferentially drives expression of a gene product in heart tissue. In some embodiments, a mammalian Anf1 promoter preferentially drive expression of a gene product in cardiac atrial cells. In some embodiments, the disclosure provides a nucleic acid comprising a tissue-specific mammalian Anf1 promoter (e.g., a human Anf1 promoter or a mouse Anf1 promoter) operably linked to a nucleic acid sequence (e.g., a transgene encoding a protein, such as a therapeutic protein, e.g., G628S KCNH2). As used herein, “tissue-specific regulatory element”, “tissue-specific promoter”, or “tissue-specific enhancer” refers to a DNA region that preferentially regulates (e.g., drives or up-regulates) gene expression in a particular cell type relative to other cell types. In some embodiments, a cell-type-specific regulatory element is specific for any cell type, such as heart cells (e.g., atrial cells), central nervous system (CNS) cells, liver cells (e.g., hepatocytes), muscle cells, etc. In some embodiments, a tissue-specific regulatory element is an atrial cell-specific regulatory element. In some embodiments, the mammalian Anf1 promoter (or Anf1 promoter variant) is tissue-specific to heart cells (e.g., atrial cells).

In some embodiments, the nucleic acid sequence encoding truncated G628S KCNH2 is operably linked to a hybrid regulatory element. In certain embodiments, a hybrid regulatory element (e.g., a CMV-e/Anf1-p) is configured to drive high levels of tissue-specific expression of a gene product (e.g., truncated G628S KCNH2 in atrial cells). In some embodiments, the hybrid regulatory element (e.g., CMV-e/Anf1-p) comprises a sequence that is set forth in SEQ ID NOs: 5 or 9. In some embodiments, the hybrid regulatory element (e.g., CMV-e/Anf1-p) consists of the sequence set forth in SEQ ID NOs: 5 or 9.

In some embodiments, the hybrid regulatory element nucleic acid (e.g., CMV-e/Anf1-p) comprises the sequence set forth in SEQ ID NOs: 5 or 9. In some embodiments, the hybrid regulatory element nucleic acid (e.g., CMV-e/Anf1-p) nucleic acid has a sequence having at least 10% sequence identity, at least 20% sequence identity, at least 30% sequence identity, at least 40% sequence identity, at least 50% sequence identity, at least 55% sequence identity, at least 60% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, or 100% sequence identity with SEQ ID NOs: 5 or 9. In certain embodiments, the hybrid regulatory element nucleic acid (e.g., CMV-e/Anf1-p) has a sequence having 100% sequence identity with SEQ ID NOs: 5 or 9. In some embodiments, the promoter of the hybrid regulatory element nucleic acid (e.g., Anf1-p) comprises the sequence set forth in SEQ ID NOs: 3 or 8. In some embodiments, the promoter of the hybrid regulatory element nucleic acid (e.g., Anf1-p) has a sequence having at least 10% sequence identity, at least 20% sequence identity, at least 30% sequence identity, at least 40% sequence identity, at least 50% sequence identity, at least 55% sequence identity, at least 60% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, or 100% sequence identity with SEQ ID NOs: 3 or 8. In certain embodiments, the promoter of the hybrid regulatory element nucleic acid (e.g., Anf1-p) has a sequence having 100% sequence identity with SEQ ID NOs: 3 or 8. In some embodiments, the enhancer of the hybrid regulatory element nucleic acid (e.g., CMV-e) comprises the sequence set forth in SEQ ID NO: 4. In some embodiments, the enhancer of the hybrid regulatory element nucleic acid (e.g., CMV-e) has a sequence having at least 10% sequence identity, at least 20% sequence identity, at least 30% sequence identity, at least 40% sequence identity, at least 50% sequence identity, at least 55% sequence identity, at least 60% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, or 100% sequence identity with SEQ ID NO: 4. In certain embodiments, the enhancer of the hybrid regulatory element nucleic acid (e.g., CMV-e) has a sequence having 100% sequence identity with SEQ ID NO: 4. In certain embodiments, a hybrid regulatory element nucleic acid (e.g., CMV-e/Anf1-p) has unexpectedly beneficial performance in conferring high levels of transgene expression in heart cells (e.g., atrial cells).

In some embodiments, a “regulatory element” comprises both promoter and enhancer elements. A “promoter” refers to a DNA sequence recognized by the synthetic machinery of the cell, or introduced synthetic machinery, required to initiate the specific transcription of a gene. An “enhancer” refers to a DNA sequence recognized by proteins that work to enhance transcription of the gene to which it is linked (e.g., transcription factors). The phrases “operably linked”, “operatively positioned”, “under control”, or “under transcriptional control” mean that the regulatory element (e.g., promoter and/or enhancer) is in the correct location and orientation in relation to the nucleic acid to control RNA polymerase initiation and expression of the transgene to which it is linked.

In some embodiments, the hybrid regulatory element is engineered such that it comprises a promoter and an enhancer element derived from different sources. In some embodiments, the hybrid regulatory element comprises a mammalian promoter and a viral enhancer (e.g., SEQ ID NOs: 5 and 9). In some embodiments, the mammalian promoter is derived from a mouse or human promoter. In some embodiments, the viral enhancer is derived from a human cytomegalovirus (CMV) enhancer. In some embodiments, the hybrid regulatory element comprises a viral promoter and a mammalian enhancer.

In some embodiments, the regulatory element (e.g., promoter or enhancer) imparts tissue-specific gene expression capabilities. In some cases, the tissue-specific regulatory sequences bind tissue-specific transcription factors that induce transcription in a tissue-specific manner. Such tissue-specific regulatory sequences (e.g., promoters, enhancers, etc.) are well known in the art.

In some embodiments, the hybrid regulatory element is inducible. Inducible regulatory elements allow regulation of gene expression and can be regulated by exogenously supplied compounds, environmental factors such as temperature, or the presence of a specific physiological state, e.g., acute phase, a particular differentiation state of the cell, or in replicating cells only. Inducible regulatory elements and inducible systems are available from a variety of commercial sources including, without limitation, Invitrogen, Clontech, and Ariad. Many other systems have been described and can be readily selected by one of skill in the art. Examples of inducible regulatory elements include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible regulatory elements which may be useful in this context are those which are regulated by a specific physiological state, e.g., temperature, acute phase, a particular differentiation state of the cell, or in replicating cells only.

### Isolated Nucleic Acids

Aspects of the disclosure provide nucleic acids (e.g., isolated nucleic acids) encoding the truncated dominant-negative KCNH2 proteins described herein. Additionally, some aspects of the disclosure provide nucleic acids (e.g., isolated nucleic acids) comprising the hybrid atrial-specific regulatory elements described herein. In some embodiments, the disclosure provides nucleic acids (e.g., isolated nucleic acids) comprising a hybrid atrial-specific regulatory element described herein operably linked to a transgene encoding a truncated dominant-negative KCNH2 protein described herein.

A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the disclosure relates to a nucleic acid sequence encoding a truncated dominant-negative KCNH2 (e.g., truncated G628S KCNH2). In some embodiments, the nucleic acid sequence encoding truncated dominant-negative KCNH2 (e.g., truncated G628S KCNH2) is codon-optimized (e.g., codon-optimized for expression in mammalian cells, such as human cells). In some embodiments, an isolated nucleic acid encoding truncated dominant-negative KCNH2 comprises at least 50%, at least 55%, at least 60%, at least 65%, at least 70%, at least 75%, at least 80%, at least 85%, at least 90%, at least 95%, at least 96%, at least 97%, at least 98%, at least 99%, at least 99.9%, or 100% sequence identity with SEQ ID NO: 2. In some embodiments, the nucleic acid sequence encoding truncated dominant-negative KCNH2 comprises up to 20 nucleotides that are different from the sequence set forth in SEQ ID NO: 2. In some embodiments, the gene encoding truncated dominant-negative KCNH2 comprises more than 20 nucleotides that are different from the sequence set forth in SEQ ID NO: 2.

In some embodiments, the nucleic acid sequence encoding truncated dominant-negative KCNH2 comprises insertions relative to SEQ ID NO: 2. In some embodiments, the nucleic acid sequence encoding truncated dominant-negative KCNH2 comprises insertions relative to SEQ ID NO: N that do not introduce a frameshift mutation. In some embodiments, an insertion in the nucleic acid sequence relative to SEQ ID NO: 2 involves the insertion of multiples of 3 nucleotides (e.g., 3, 6, 9, 12, 15, 18, etc.). In some embodiments, an insertion in the nucleic acid sequence relative to SEQ ID NO: 2 leads to an increase in the total number of amino acid residues in the resultant truncated dominant-negative KCNH2 (e.g., an increase of 1-3, 1-5, 3-10, 5-10, 5-15, or 10-20 amino acid residues).

In some embodiments, the nucleic acid sequence encoding truncated dominant-negative KCNH2 comprises deletions relative to SEQ ID NO: 2. In some embodiments, the nucleic acid sequence encoding truncated dominant-negative KCNH2 comprises deletions relative to SEQ ID NO: 2 that do not introduce a frameshift mutation. In some embodiments, a deletion in the nucleic acid sequence relative to SEQ ID NO: 2 involves the deletion of multiples of 3 nucleotides (e.g., 3, 6, 9, 12, 15, 18, etc.). In some embodiments, a deletion in the nucleic acid encoding truncated dominant-negative KCNH2 leads to a decrease in the total number of amino acid residues in the resultant truncated dominant-negative KCNH2 protein (e.g., a decrease of 1-3, 1-5, 3-10, 5-10, 5-15, or 10-20 amino acid residues).

In some embodiments, the nucleic acid sequence encoding truncated dominant-negative KCNH2 is a codon-optimized sequence (e.g., codon-optimized for expression in mammalian cells). In some embodiments, a codon-optimized sequence encoding truncated dominant-negative KCNH2 comprises reduced GC content relative to a wild-type sequence that has not been codon-optimized. In some embodiments, a codon-optimized sequence encoding truncated dominant-negative KCNH2 comprises a 1-5%, 3-5%, 3-10%, 5-10%, 5-15%, 10-20%, 15-30%, 20-40%, 25-50%, or 30-60% reduction in GC content relative to a wild-type sequence that has not been codon-optimized. In some embodiments, a codon-optimized sequence encoding truncated dominant-negative KCNH2 comprises fewer guanine and/or cytosine nucleobases relative to a wild-type sequence that has not been codon-optimized. In some embodiments, a codon-optimized sequence encoding truncated dominant-negative KCNH2 comprises fewer CpG dinucleotide islands relative to a wild-type sequence that has not been codon-optimized. In some embodiments, a codon-optimized sequence encoding truncated dominant-negative KCNH2 comprises 1-3, 3-5, 3-10, 5-10, 5-15, 10-20, 15-30, 20-40, 25-50, or 30-60 fewer CpG dinucleotide islands relative to a wild-type sequence that has not been codon-optimized.

In some aspects, the disclosure provides nucleic acids (e.g., isolated nucleic acids) comprising a transgene operably linked to a hybrid regulatory element as described herein. For example, in some embodiments, a nucleic acid comprises a transgene operably linked to a regulatory element comprising (i) an atrial natriuretic peptide (Anf) promoter and (ii) an enhancer element, wherein the enhancer element is not a naturally-occurring Anf enhancer element. In some embodiments, a nucleic acid comprises a transgene operably linked to a regulatory element comprising an atrial natriuretic peptide (Anf) promoter. In some embodiments, a nucleic acid comprises a transgene operably linked to a regulatory element comprising an enhancer element, wherein the enhancer element is not a naturally-occurring Anf enhancer element. In some embodiments, the transgene encodes any protein or nucleic acid that would derive benefit from being delivered preferentially to cardiac tissue. For example, in some embodiments, the transgene encodes KCNH2, Connexin 40, or Connexin 43.

In some embodiments, the nucleic acid sequence encoding truncated dominant-negative KCNH2 is operably linked to a hybrid regulatory element. In some embodiments, the nucleic acid sequence encoding truncated dominant-negative KCNH2 is operably linked to a CMV-e/Anf1-p hybrid regulatory element as described herein (e.g., SEQ ID NOs: 14 or 15). In some embodiments, an isolated nucleic acid encoding truncated dominant-negative KCNH2 driven by a CMV-e/Anf1-p hybrid regulatory element comprises at least 10%, at least 20%, at least 30%, at least 40%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.9%, or 100% sequence identity with SEQ ID NOs: 14 or 15. In some embodiments, an isolated nucleic acid encoding truncated dominant-negative KCNH2 driven by a CMV-e/Anf1-p hybrid regulatory element comprises up to 20 nucleotides that are different from the sequences set forth in SEQ ID NOs: 14 or 15. In some embodiments, an isolated nucleic acid encoding truncated dominant-negative KCNH2 driven by a CMV-e/Anf1-p hybrid regulatory element comprises more than 20 nucleotides that are different from the sequences set forth in SEQ ID NOs: 14 or 15.

In some aspects, the disclosure relates to isolated nucleic acids comprising a transgene (e.g., truncated G628S KCNH2) operably linked to a regulatory element via a chimeric intron. In some embodiments, the intronic sequence ranges from about 100 to 120 (e.g., 100, 101, 102, 103, 104, 105, 106, 107, 108, 109, 110, 111, 112, 113, 114, 115, 116, 117, 118, 119, or 120) nucleotides in length. In some embodiments, a chimeric intron is adjacent to one or more untranslated sequences (e.g., an untranslated sequence located between the promoter sequence and the chimeric intron sequence and/or an untranslated sequence located between the chimeric intron and the first codon of the transgene sequence). In some embodiments, each of the one or more untranslated sequences are non-coding sequences from a rabbit beta-globulin gene (e.g., untranslated sequence from rabbit beta-globulin exon 1, exon 2, etc.).

Some nucleic acids further comprise additional expression control sequences such as appropriate transcription initiation and termination sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. In some embodiments, a nucleic acid regulatory element comprises one or more expression control sequences (e.g., transcription initiation and termination sequences;

efficient RNA processing signals such as splicing and polyA signals; sequences that stabilize cytoplasmic mRNA; sequences that enhance translation efficiency (e.g., Kozak consensus sequence); sequences that enhance protein stability; and/or sequences that enhance secretion of the encoded product).

For nucleic acids encoding proteins, a polyadenylation sequence generally is inserted following the transgene sequences and before the 3′ AAV ITR sequence. In some embodiments, an rAAV construct useful in the present disclosure also contains an intron, desirably located between the promoter/enhancer sequence and the transgene. One possible intron sequence is derived from SV-40 and is referred to as the SV-40 T intron sequence.

In some embodiments, a nucleic acid comprises an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional, and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931.; and Klump, H et al., Gene Therapy, 2001; 8:811-817).

Recombinant Adeno-Associated Viruses (rAAVs)

In some embodiments, isolated nucleic acids of the disclosure are recombinant adeno-associated virus (rAAVs) vectors. rAAV vectors of the disclosure are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule or other gene product, of interest. The nucleic acid coding sequence is operatively linked to regulatory components in a manner which permits transgene transcription, translation, and/or expression in a cell of a target tissue.

In some embodiments, an isolated nucleic acid as described by the disclosure comprises a region (e.g., a first region) comprising a first adeno-associated virus (AAV) inverted terminal repeat (ITR), or a variant thereof and a second region comprising a transgene encoding truncated dominant-negative KCNH2. In some embodiments, the isolated nucleic acid (e.g., the recombinant AAV vector) is packaged into a capsid protein and administered to a subject and/or delivered to a selected target cell. In some embodiments, the transgene comprises a region encoding, for example, a protein and/or an expression control sequence (e.g., a poly-A tail), as described elsewhere in the disclosure.

The instant disclosure provides a vector comprising a single, cis-acting wild-type ITR. In some embodiments, the ITR is a 5′ ITR. In some embodiments, the ITR is a 3′ ITR. Generally, ITR sequences are about 145 bp in length. Preferably, substantially the entire sequences encoding the ITR(s) is used in the molecule, although some degree of minor modification of these sequences is permissible. In some embodiments, an ITR is mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. In some embodiments, AAV ITR sequences are obtained from any known AAV, including presently identified mammalian AAV types. In some embodiments, an ITR sequence is an AAV1, AAV2, AAV3, AAV4, AAV5, AAV6, AAV7, AAV8, AAVrh8, AAV9, AAV10, and/or AAVsrh10 ITR sequence.

In some embodiments, a rAAV vector comprises a nucleic acid sequence encoding a truncated dominant-negative KCNH2 protein or a portion thereof (e.g., under the control of a hybrid regulatory element (e.g., CMV-e/Anf1-p)). In some embodiments, the rAAV vector is a self-complementary vector that comprises a nucleic acid sequence encoding a truncated dominant-negative KCNH2 protein or portion thereof.

In some embodiments, the isolated nucleic acids and/or rAAVs of the present disclosure are modified and/or selected to enhance the targeting of the isolated nucleic acids and/or rAAVs to a target tissue (e.g., heart tissue). Non-limiting methods of modifications and/or selections include AAV capsid serotypes (e.g., AAV1), tissue-specific promoters, and/or targeting peptides. In some embodiments, the isolated nucleic acids and rAAVs of the present disclosure comprise AAV capsid serotypes with enhanced targeting to heart tissues (e.g., AAV1). In some embodiments, the isolated nucleic acids and rAAVs of the present disclosure comprise tissue-specific regulatory elements. In some embodiments, the isolated nucleic acids and rAAVs of the present disclosure comprise AAV capsid serotypes with enhanced targeting to heart tissue and tissue-specific regulatory elements.

In some aspects, the disclosure provides isolated AAVs. As used herein with respect to AAVs, the term “isolated” refers to an AAV that has been artificially obtained or produced. In some embodiments, isolated AAVs are produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises an AAV1, AAV2, AAV6, AAV8, or AAV9 capsid protein, or a protein having substantial homology thereto. In some embodiments, the rAAV comprises an AAV1 capsid protein.

In some embodiments, AAV variants described herein are useful for delivering gene therapy to cardiac cells (e.g., heart tissue, e.g., the atrium of the heart). Accordingly, in some embodiments, AAV variants described herein are useful for the treatment of cardiovascular disorders. As used herein, a “cardiovascular disorder” is a disease or condition of the cardiovascular system. In some embodiments, a cardiovascular disease affects the heart, circulatory system, arteries, veins, blood vessels and/or capillaries. In some embodiments, a cardiovascular disorder is of a genetic origin, either inherited or acquired through a somatic mutation. Non-limiting examples of cardiovascular disorders include rheumatic heart disease, valvular heart disease, hypertensive heart disease, aneurysm, atherosclerosis, hypertension (e.g., high blood pressure), peripheral arterial disease (PAD), ischemic heart disease, angina, coronary heart disease, coronary artery disease, myocardial infarction, cerebral vascular disease, transient ischemic attack, inflammatory heart disease, cardiomyopathy, pericardial disease, congenital heart disease, heart failure, stroke, and myocarditis due to Chagas disease.

In some embodiments, the rAAVs of the disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeats (ITRs) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs are useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US Patent Application Publication Number US 2003/0138772, the contents of which are incorporated herein by reference in their entirety). Typically, the methods involve culturing a host cell which contains a nucleic acid sequence encoding an AAV capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. Typically, capsid proteins are structural proteins encoded by the cap gene of an AAV. In some embodiments, AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, capsid proteins protect a viral genome, deliver a genome and/or interact with a host cell. In some aspects, capsid proteins deliver the viral genome to a host in a tissue specific manner.

In some embodiments, the AAV capsid protein is of an AAV serotype selected from the group consisting of AAV1, AAV2, AAV6, AAV8, and AAV9. In some embodiments, the AAV capsid protein is of an AAV1 serotype. In some embodiments, the AAV capsid protein is of an AAV2 serotype. In some embodiments, the AAV capsid protein is of an AAV6 serotype. In some embodiments, the AAV capsid protein is of an AAV8 serotype. In some embodiments, the AAV capsid protein is of an AAV9 serotype.

In certain embodiments, the disclosure relates to rAAV vectors comprising artificial transcription elements. As used here, the term “artificial transcription element” refers, in some embodiments, to a synthetic sequence enabling the controlled transcription of DNA by an RNA polymerase to produce an RNA transcript. Transcriptionally active elements of the present disclosure are generally smaller than 500 bp, preferably smaller than 200 bp, more preferably smaller than 100, most preferably smaller than 50 bp. In some embodiments, an artificial transcription element comprises two or more nucleic acid sequences from transcriptionally active elements. Transcriptionally active elements are generally recognized in the art and include, for example, promoter, enhancer sequence, TATA box, G/C box, CCAAT box, specificity protein 1 (Sp1) binding site, Inr region, CRE (CAMP regulatory element), activating transcription factor 1 (ATF1) binding site, ATF1-CRE binding site, APBβ box, APBα box, CArG box, CCAC box and those disclosed by U.S. Pat. No. 6,346,415. Combinations of the foregoing transcriptionally active elements are also contemplated.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

In some embodiments, components to be cultured in the host cell to package a rAAV vector in an AAV capsid are provided to the host cell in trans. Alternatively, in some embodiments, any one or more of the required components (e.g., recombinant AAV vector, rep sequences, cap sequences, and/or helper functions) are provided by a stable host cell which has been engineered to contain one or more of the required components using methods known to those of skill in the art. Most suitably, such a stable host cell will contain the required component(s) under the control of an inducible promoter. However, in some embodiments, the required component(s) is under the control of a constitutive promoter. Examples of suitable inducible and constitutive promoters are provided herein, in the discussion of regulatory elements suitable for use with the transgene. In still another alternative, a selected stable host cell contains selected component(s) under the control of a constitutive promoter and other selected component(s) under the control of one or more inducible promoters. For example, in some embodiments, a stable host cell is generated which is derived from 293 cells (which contain E1 helper functions under the control of a constitutive promoter), but which contain the rep and/or cap proteins under the control of inducible promoters. In other embodiments, still other stable host cells are generated by one of skill in the art.

In some embodiments, the disclosure relates to a host cell that comprises a nucleic acid comprising a sequence set forth in SEQ ID NO: 2 that is operably linked to a regulatory element. In some embodiments, the disclosure relates to a composition comprising the host cell described above. In some embodiments, the composition comprising the host cell above further comprises a cryopreservative.

In some embodiments, the recombinant AAV vector, rep sequences, cap sequences, and helper functions useful for producing the rAAV of the disclosure are delivered to the packaging host cell using any appropriate genetic element (vector). In some embodiments, the selected genetic element is delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs are produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell (e.g., a non-human primate, rodent, or human cell). In some embodiments, the host cell is a mammalian cell, a yeast cell, a bacterial cell, an insect cell, a plant cell, or a fungal cell. In some embodiments, a host cell is used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a regulatory element. The term “expression vector” or “construct” means any type of genetic construct containing a nucleic acid in which part or all of the nucleic acid encoding sequence is capable of being transcribed. In some embodiments, expression includes transcription of the nucleic acid, for example, to generate a biologically active polypeptide product or inhibitory RNA (e.g., shRNA, miRNA, miRNA inhibitor) from a transcribed gene.

The foregoing methods for packaging recombinant vectors in desired AAV capsids to produce the rAAVs of the disclosure are not meant to be limiting and other suitable methods will be apparent to the skilled artisan.

rAAV Vector: Transgene Coding Sequences

In some embodiments, the disclosure provides an rAAV comprising a transgene encoding a truncated dominant-negative KCNH2 protein. Also contemplated herein are methods of treating atrial fibrillation by delivering a transgene to a subject using the rAAVs described herein. In some embodiments, the disclosure relates to a method for treating atrial fibrillation, the method comprising administering a rAAV to a subject. In some embodiments, the rAAV comprises a hybrid regulatory element. In some embodiments, the rAAV comprises a chimeric intron. In some embodiments, the rAAV comprises an artificial transcription element. In some embodiments, the promoter, chimeric intron, or artificial transcription element is operably linked to a transgene. In some embodiments, the transgene encodes a truncated dominant-negative KCNH2 protein.

In some embodiments, one or more bindings sites for one or more of miRNAs are incorporated in a transgene of a rAAV vector, to inhibit the expression of the transgene in one or more tissues of a subject harboring the transgene. The skilled artisan will appreciate that in some embodiments, binding sites are selected to control the expression of a transgene in a tissue specific manner. For example, in some embodiments, binding sites for the liver-specific miR-122 is incorporated into a transgene to inhibit expression of that transgene in the liver. In some embodiments, the target sites in the mRNA are in the 5′ UTR, the 3′ UTR, or in the coding region. Typically, the target site is in the 3′ UTR of the mRNA. Furthermore, in some embodiments, the transgene is designed such that multiple miRNAs regulate the mRNA by recognizing the same or multiple sites. In some embodiments, the presence of multiple miRNA binding sites results in the cooperative action of multiple RISCs and provide highly efficient inhibition of expression. In some embodiments, the target site sequence comprises a total of 5-100, 10-60, or more nucleotides. In some embodiments, the target site sequence comprises at least 5 nucleotides of the sequence of a target gene binding site.

In some embodiments, a transgene comprises one or more (e.g., 1, 2, 3, 4, 5, or more) miRNA binding sites that de-target expression of a transgene from immune cells (e.g., antigen presenting cells (APCs), such as macrophages, dendrites, etc.). Incorporation of miRNA binding sites for immune-associated miRNAs may de-target transgene (e.g., one or more inhibitory nucleic acids) expression from APCs and thus reduce or eliminate immune responses (cellular and/or humoral) produced in the subject against products of the transgene, for example, as described in US 2018/0066279, the entire contents of which are incorporated herein by reference.

rAAV Administration Methods

In some embodiments, the rAAVs are delivered to a subject in compositions according to any appropriate methods known in the art. In some embodiments, the rAAV, preferably suspended in a physiologically compatible carrier (i.e., in a composition), is administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

In some embodiments, delivery of the rAAVs to a mammalian subject is by, for example, intramuscular injection or by administration into the bloodstream of the mammalian subject. In some embodiments, administration into the bloodstream is via injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

In some embodiments, a method for delivering a transgene to atrial cells in a subject comprises administering rAAV by a single route or by multiple routes. In some embodiments, the rAAVs are delivered via direct “gene painting” as described in U.S. Patent Publication 2012/0251498, the entire contents of which is incorporated herein by reference. This method applies a virus with poloxamer directly onto atria, resulting in diffuse epicardial gene transfer with negligible penetration into other regions of the heart. In some embodiments, a method for delivering a transgene to atrial cells in a subject comprises co-administering an effective amount of rAAV by two different administration routes, e.g., by gene painting and by administration into the bloodstream. In some embodiments, co-administration is performed at approximately the same time, or at different times.

Aspects of the disclosure relate to compositions comprising a recombinant AAV comprising at least one modified genetic regulatory sequence or element. In some embodiments, the composition further comprises a pharmaceutically acceptable carrier.

In some embodiments, the compositions of the disclosure comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some aspects, the disclosure relates to a composition (e.g., a pharmaceutical composition) comprising an rAAV comprising a nucleic acid encoding a truncated dominant-negative KCNH2 protein, optionally under the control of a hybrid regulatory element (e.g., CMV-e/Anf1-p).

In some embodiments, suitable carriers are readily selected by one of skill in the art in view of the indication for which the rAAV is directed. For example, in some embodiments, one suitable carrier includes saline, which is optionally formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure contain, in some embodiments, other conventional pharmaceutical ingredients, such as preservatives, or chemical stabilizers in addition to the rAAV and carrier(s). Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. In some embodiments, routes of administration are combined, if desired.

The dose of rAAV virions required to achieve a particular “therapeutic effect.” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of 4.68×10⁷ is appropriate. In some embodiments, a dosage of 4.68×10⁸ genome copies is appropriate. In some embodiments, a dosage of 4.68×10⁹ genome copies is appropriate. In some embodiments, a dosage of 1.17×10¹⁰ genome copies is appropriate. In some embodiments, a dosage of 2.34×10¹⁰ genome copies is appropriate. In some embodiments, a dosage of 3.20×10¹¹ genome copies is appropriate. In some embodiments, a dosage of 1.2×10¹³ genome copies is appropriate. In some embodiments, a dosage of about 1×10¹⁴ vector genome (vg) copies is appropriate.

In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, and any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations contain, in some embodiments, at least about 0.1% of the active compound or more, although the percentage of the active ingredient(s) may, of course, be varied and may conveniently be between about 1 or 2% and about 70% or 80% or more of the weight or volume of the total formulation. Naturally, in some embodiments, the amount of active compound in each therapeutically useful composition is prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, in some embodiments, a variety of dosages and treatment regimens is desirable.

In certain circumstances it will be desirable to deliver the rAAV-based therapeutic constructs in suitably formulated pharmaceutical compositions disclosed herein either intravenously, intramuscularly, or orally, intraperitoneally. In some embodiments, the administration modalities as described in U.S. Pat. Nos. 5,543,158; 5,641,515 and 5,399,363 (each specifically incorporated herein by reference in its entirety) are to deliver the rAAVs.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. In some embodiments, proper fluidity is maintained, for example, using a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. In some embodiments, for example, one dosage is dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein are also formulated, in some embodiments, in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a host.

In some embodiments, delivery vehicles such as liposomes, nanocapsules, microparticles, microspheres, lipid particles, vesicles, and the like, are used for the introduction of the compositions of the present disclosure into suitable host cells. In particular, in some embodiments, the rAAV vector delivered transgenes are formulated for delivery either encapsulated in a lipid particle, a liposome, a vesicle, a nanosphere, or a nanoparticle or the like.

In some embodiments, such formulations are preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core.

Alternatively, in some embodiments, nanocapsule formulations of the rAAV are used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the disclosure relates to administration of one or more additional therapeutic agents to a subject who has been administered an rAAV or pharmaceutical composition as described herein.

### Methods of Delivery to Cardiac Tissue

Aspects of the present disclosure provide methods for treating cardiac diseases or disorders such as Atrial Fibrillation (AF). AF is the most common sustained cardiac arrhythmia, affecting an estimated 33.5 million people worldwide. AF causes irregular and often abnormally fast heart rate, leading to increased risk of heart failure, stroke, dementia, and death. A main underlying mechanism for AF includes triggering events that start the arrhythmia, and maintenance conditions that sustain the abnormal rhythm. Triggers generally are single or repetitive atrial premature beats that occur when depolarizations in individual cells spread throughout the atria. A variety of mechanisms can sustain AF, including continuation of the triggering after depolarizations, and either focal or broad reentrant electric circuits.

Accordingly, in some embodiments, the disclosure provides isolated nucleic acids, rAAVs, compositions, and methods useful in treating cardiac diseases or disorders such as AF. In some embodiments, the isolated nucleic acids, rAAVs, compositions, and methods are for the treatment of AF. In some embodiments, methods for treating cardiac diseases or disorders such as AF in a subject comprise administering an isolated nucleic acid, an rAAV, or a composition as described herein that comprises a transgene encoding truncated dominant-negative KCNH2, optionally under the control of a hybrid regulatory element that confers atrial-specific transgene expression (e.g., CMV-e/Anf1-p).

In some aspects, the disclosure provides a method of inhibiting the delayed rectifier potassium current (IKr) channel function of wild-type KCNH2 channels in atrial cells of a subject, comprising administering the isolated nucleic acids, rAAVs, or the compositions described herein to a subject having or suspected of having cardiac disease or disorder such as AF. As used herein, a subject having or suspected of having AF, in some embodiments, exhibits chest pain, dizziness, fatigue, lightheadedness, reduced ability to exercise, shortness of breath, weakness, and/or heart palpitations.

In some embodiments, delivery of a truncated dominant-negative KCNH2 protein to a cell or subject (e.g., via an rAAV) functions to decrease the activity of wild-type KCNH2 protein in the cell or subject. In some embodiments, the activity of wild-type KCNH2 protein is decreased by at least 10%, 20%, 30%, 40%, 50%, 60%, 70%, 80%, 90%, or more, relative to wild-type KCNH2 protein.

In some embodiments, administering the isolated nucleic acids, the rAAVs, or the compositions described herein to a subject promotes expression of truncated dominant-negative KCNH2 by between 2-fold and 100-fold (e.g., 2-fold, 5-fold, 10-fold, 20-fold, 50-fold, 75-fold, 100-fold, etc.) compared to a control subject. In some embodiments, administering the isolated nucleic acids, the rAAVs, or the compositions described herein to a subject promotes expression of truncated dominant-negative KCNH2 in heart tissue of a subject by between 2-fold and 100-fold (e.g., 2-fold, 5-fold, 10-fold, 20-fold, 50-fold, 75-fold, 100-fold, etc.) compared to a control subject. As used herein, in some embodiments, a “control” subject refers to a subject that is not administered the isolated nucleic acids, the rAAVs, or the compositions described herein; or a healthy subject. In some embodiments, a control subject is the same subject that is administered the isolated nucleic acids, the rAAVs, or the compositions described herein (e.g., prior to the administration). In some embodiments, administering the isolated nucleic acids, the rAAVs, or the compositions described to a subject promotes expression of truncated dominant-negative KCNH2 by 2-fold compared to a control. In some embodiments, administering the isolated nucleic acids, the rAAVs, or the compositions described to a subject promotes expression of truncated dominant-negative KCNH2 by 100-fold compared to a control. In some embodiments, administering the isolated nucleic acids, the rAAVs, or the compositions described to a subject promotes expression of truncated dominant-negative KCNH2 by 5-fold compared to a control. In some embodiments, administering the isolated nucleic acids, the rAAVs, or the compositions described to a subject promotes expression of truncated dominant-negative KCNH2 by 10-fold compared to a control. In some embodiments, administering the isolated nucleic acids, the rAAVs, or the compositions described to a subject promotes expression of truncated dominant-negative KCNH2 by 5-fold to 100-fold compared to control (e.g., 5-fold to 10-fold, 10-fold to 15-fold, 10-fold to 20-fold, 15-fold to 25-fold, 20-fold to 30-fold, 25-fold to 35-fold, 30-fold to 40-fold, 35-fold to 45-fold, 40-fold to 60-fold, 50-fold to 75-fold, 60-fold to 80-fold, 75-fold to 100-fold compared to a control).

In some embodiments, administering the isolated nucleic acids, the rAAVs, or the compositions described herein to a subject promotes expression of truncated dominant-negative KCNH2 in a subject (e.g., promotes expression of truncated dominant-negative KCNH2 in the heart of a subject) by between a 5% and 200% increase (e.g., 5-50%, 25-75%, 50-100%, 75-125%, 100-200%, or 100-150% etc.) compared to a control subject.

In some aspects, the disclosure provides a method of treating a subject having a cardiac disease or disorder such as AF, the method comprising administering to the subject an effective amount of an rAAV comprising a capsid containing a nucleic acid engineered to express truncated dominant-negative KCNH2 in the atria of the subject.

In some aspects, the disclosure provides a method of increasing the expression of a protein in an atrial cardiomyocyte comprising administering a nucleic acid comprising a hybrid regulatory element as described herein to the atrial cardiomyocyte. In some embodiments, the expression of the protein is increased relative to a control cardiomyocyte (e.g., increased by at least 10%, 20%, 30%, 50%, 75%, or 100% relative to the control cardiomyocyte). In some embodiments, the expression of the protein is higher in the atrial cardiomyocyte relative to a control cell in the subject, optionally wherein the control cell is a ventricular cardiomyocyte or a liver cell from the same subject.

As used herein, the term “treating” refers to the application or administration of a composition (e.g., an isolated nucleic acid or rAAV as described herein) to a subject who has a cardiac disease or disorder such as AF with the purpose to cure, heal, alleviate, relieve, alter, remedy, ameliorate, improve, or affect the disorder or symptoms of the cardiac disease or disorder.

Alleviating a cardiac disease or disorder includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, in some embodiments, development also refers to progression that is undetectable. For purpose of this disclosure, development or progression refers to the biological course of the symptoms. “Development” includes occurrence, recurrence, and onset.

In some embodiments, subject is a human, a mouse, a rat, a pig, a dog, a cat, or a non-human primate. In some embodiments, a subject has or is suspected of having a cardiac disease or disorder such as AF.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., to the heart, e.g., via gene painting), oral, intravenous, or intramuscular. In some embodiments, routes of administration are combined, if desired.

### EXAMPLES

In order that the disclosure described herein may be more fully understood, the following examples are set forth. The examples described in this application are offered to illustrate the methods, devices, and systems provided herein and are not to be construed in any way as limiting their scope.

### Example 1. Generation of a Functional Truncated Dominant-Negative G628S Mutant KCNH2 Protein

While the dominant-negative G628S mutation of the KCNH2 potassium channel shows promise as a treatment for atrial fibrillation, the DNA sequence of full-length KCNH2 is too long (3480 bp) to be packaged into AAV vectors when combined with regulatory elements. This example describes an exemplary truncated form of G628S KCNH2 (as shown in SEQ ID NO: 22) that retains its dominant-negative functionality while also fitting into an AAV vector.

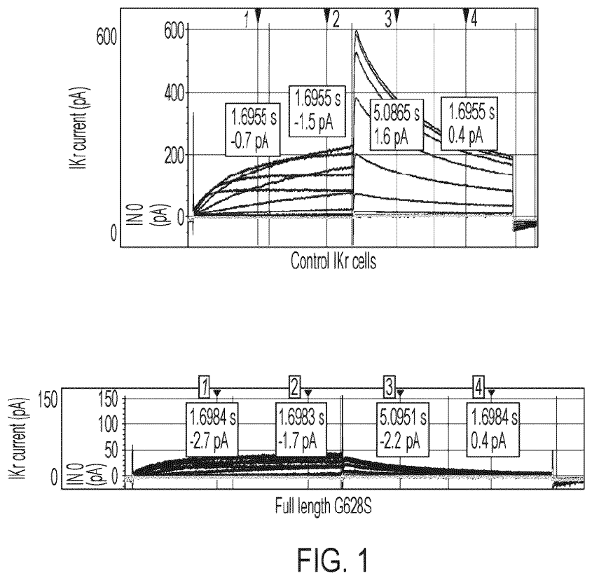

Expression and function of a plasmid encoding the exemplary truncated G628S KCNH2 (SEQ ID NO: 2) was tested by transfecting constructs encoding either full-length or truncated G628S KCNH2 into CHO cells that constitutively express wild-type KCNH2 and KCN2, the two proteins that provide delayed rectifier potassium current (IKr) channel function of wild-type potassium channels. Paired experiments were performed, using transfection of the gene for green fluorescent protein (GFP) as a control for each experiment. For the full-length G628S KCNH2 experiment, control cells showed an average sustained current of 171 pA and a tail current of 237 pA (FIG. 1, left), and transfection with full-length G628S KCNH2 resulted in an average of 65% reduction in sustained current and 58% reduction in tail current relative to the control cells (FIG. 1, middle). For the truncated G628S KCNH2 experiment, control cells showed an average sustained current of 185 pA and tail current of 190 pA, and transfection with truncated G628S KCNH2 resulted in an average of 70% reduction in sustained current and 78% reduction in tail current relative to the control cells (FIG. 1, right). Because the CHO cells intrinsically express the wild-type components of the I_{Kr} channel, the loss of current with expression of either full-length or truncated KCNH2 verifies the dominant-negative activity of this mutation. Comparison between the levels of current loss caused by the full-length or truncated channels shows that the truncated G628S KCNH2 retains similar, if not higher, channel block capabilities than the full-length G628S KCNH2 mutation.

Accordingly, as demonstrated by the data, the exemplary truncated G628S KCNH2 retained dominant-negative activity against wild-type KCNH2 in these cells.

### Example 2. Epigenetic Interrogation of the Mouse Anf Promoter Identifies Atrial-Specific Regulatory Element

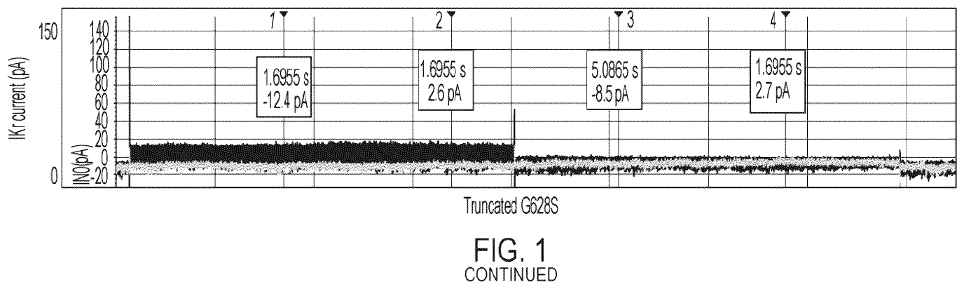

Current atrial-specific regulatory elements, such as those from the atrial natriuretic factor (Anf) and sarcolipin genes, either lack robust transgene expression or are not fully restricted to the atria. Therefore, datasets from the Encyclopedia of DNA Elements (ENCODE) were mined to delineate the native Anf regulatory element region in mouse heart, as the epigenetic and transcriptional regulatory marks are best characterized for multiple cell and tissue types in the mouse genome. The mouse genome (mm9 build) was thus queried for genome-wide DNase-hypersensitivity (DHS, DNase-seq) and histone modifications (Chromatin Immunoprecipitation-sequencing [ChIP-seq]), which are indicative of promoter and enhancer regions, respectively. Through this method, a predicted 1.58-kilobase regulatory element was identified (SEQ ID NO: 17) that defines Anf expression in the heart (FIG. 2). Based on epigenetic marks, the region was subdivided into a presumptive 0.6 kilobase enhancer (Anf-e; SEQ ID NO: 6) and a 0.97 kilobase promoter (Anf-p; SEQ ID NO: 3).

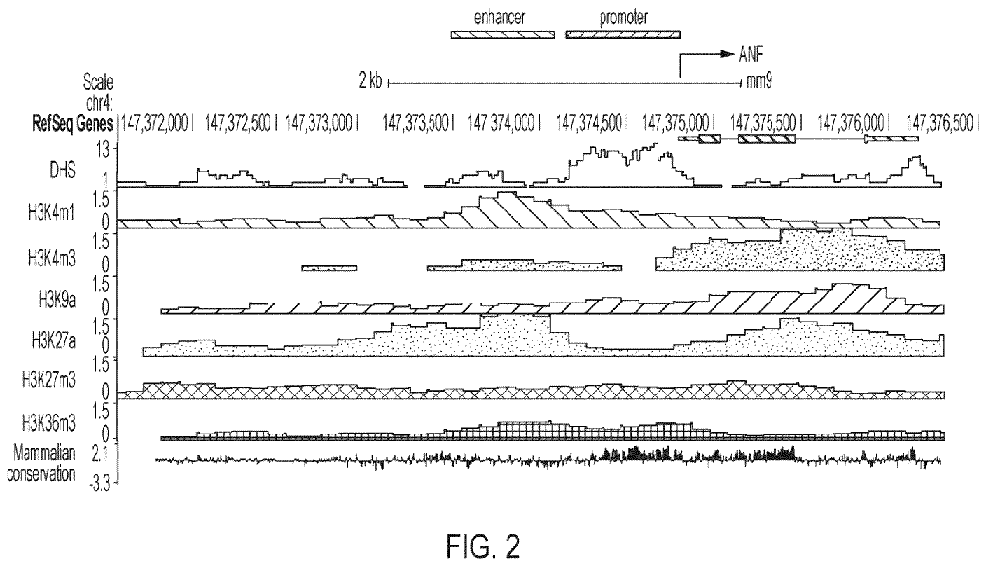

The full 1.58 kilobase Anf regulatory element was cloned into a firefly luciferase (FLuc) expression plasmid and tested against a CMV enhancer/chicken beta-actin ubiquitous promoter (CB6), which confers high levels of transgene expression but with low specificity. The 1.58-kb Anf regulatory element (ANF-FLuc) and CB6 (CB6-FLuc) constructs were transfected into ventricular cardiomyocytes (H9C2) and atrial cardiomyocytes (HL-1) and FLuc luminescence was measured as a readout of promoter activity. The 1.58-kb ANF-FLuc showed 100-fold less FLuc expression compared to CB6-FLuc. However, the 1.58-kb Anf regulatory element conferred more transgene expression in HL-1 cells than in H9C2 cells, indicating that the 1.58-kb Anf regulatory element has atrial-dependent activity (FIGS. 3A-3B).

### Example 3. Hybrid Anf-p/CMV-e Promoter Confers High Levels of Atrial-Specific Gene Expression

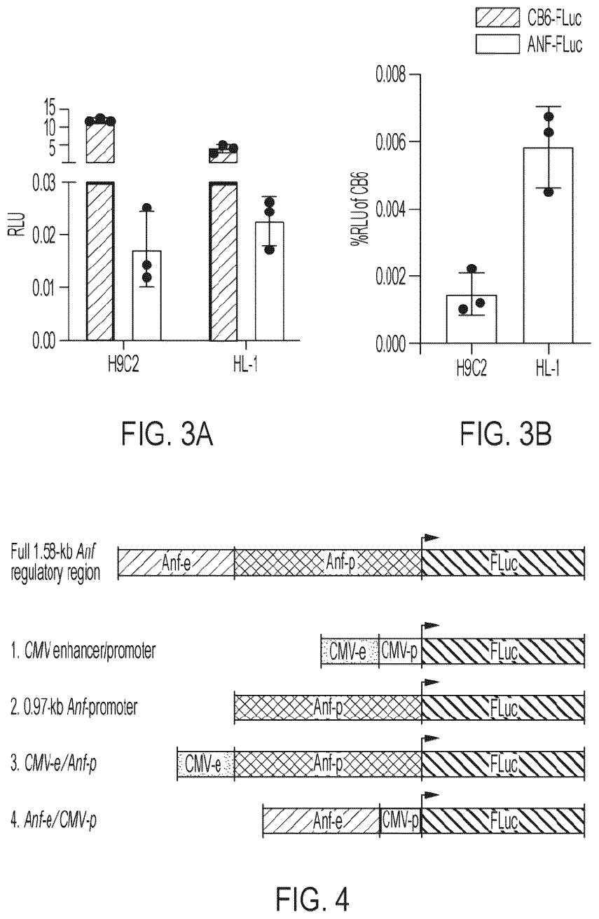

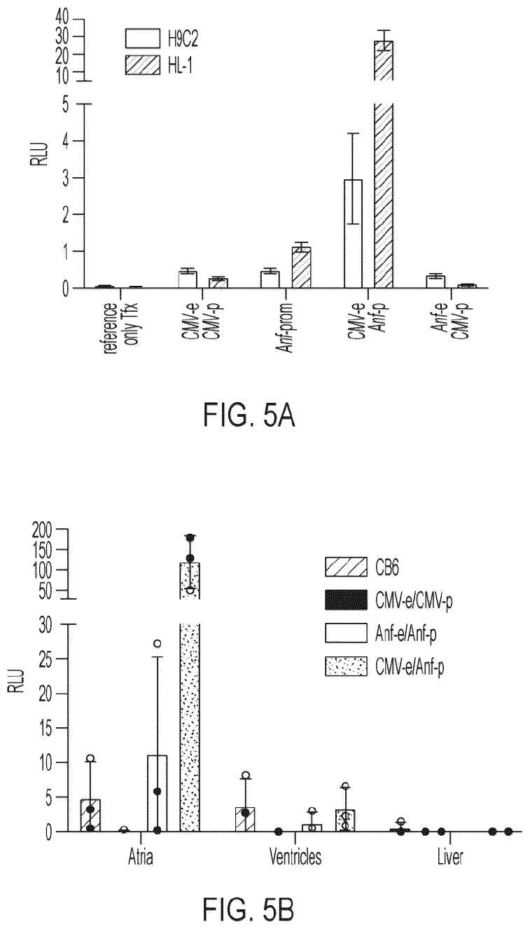

Because the 1.58-kb Anf regulatory element showed substantially less activity than the ubiquitous CB6 promoter, hybrid regulatory elements were constructed that were derived from the Anf enhancer-promoter sequences delineated in Example 2 (FIG. 2). Two hybrid Anf-derived regulatory elements were designed: (1) a construct with the CMV enhancer and the Anf promoter (CMV-e/Anf1-p; SEQ ID NO: 9), and (2) a construct with the Anf enhancer and the CMV promoter (Anf1-e/CMV-p; SEQ ID NO: 16) (FIG. 4, constructs 3 and 4, respectively). These constructs, along with a full-length CMV promoter (CMV-e/CMV-p; SEQ ID NO: 18) and a 0.97-kb Anf promoter (without the Anf-e region; SEQ ID NO: 3) (FIG. 4, constructs 1 and 2, respectively) were cloned into an FLuc expression plasmid, transfected into H9C2 and HL-1 cells, and tested for activity through measurements of FLuc luminescence. The hybrid CMV-e/Anf-p regulatory element conferred 10-fold higher transcriptional activity in HL-1 cells than in H9C2 cells (FIG. 5A). Importantly, combining the CMV-e region with Anf-p increased Anf-p activity more than 20-fold compared to Anf-p alone.

Constructs 1, 3 and 4 from FIG. 4 and a fourth construct with a CB6 promoter were packaged into AAV1 capsids to test their activity in vivo. Vectors were applied to rabbit hearts by atrial and ventricular gene painting at a dose of 1E11 vector particles (vp) per animal, and animals were sacrificed three weeks later. Heart tissue was harvested and subjected to luciferase assay as a readout of promoter activity. Liver tissue was measured as control.

As shown in FIG. 5B, the hybrid Anf-p/CMV-e regulatory element achieved high-level gene expression, out-performing all other regulatory elements, including CB6, and retained atrial selectivity.

### Representative Sequences

TABLE 1
Nucleic Acid Sequences

| SEQ | | |
|---|---|---|
| ID | | |
| NO | Name | Sequence (5' to 3') |
| 1 | Wild-type | ATGCCGGTGCGGAGGGGCCACGTCGCGCCGCAGAACACCTTCCTGGAC |
| | KCNH2 | ACCATCATCCGCAAGTTTGAGGGCCAGAGCCGTAAGTTCATCATCGCCA |
| | (truncated) | ACGCTCGGGTGGAGAACTGCGCCGTCATCTACTGCAACGACGGCTTCTG |
| | | CGAGCTGTGCGGCTACTCGCGGGCCGAGGTGATGCAGCGACCCTGCAC |
| | | CTGCGACTTCCTGCACGGGCCGCGCACGCAGCGCCGCGCTGCCGCGCA |
| | | GATCGCGCAGGCACTGCTGGGCGCCGAGGAGCGCAAAGTGGAAATCGC |
| | | CTTCTACCGGAAAGATGGGAGCTGCTTCCTATGTCTGGTGGATGTGGTG |
| | | CCCGTGAAGAACGAGGATGGGGCTGTCATCATGTTCATCCTCAATTTCG |
| | | AGGTGGTGATGGAGAAGGACATGGTGGGGTCCCCGGCTCATGACACCA |
| | | ACCACCGGGGCCCCCCCACCAGCTGGCTGGCCCCAGGCCGCGCCAAGA |
| | | CCTTCCGCCTGAAGCTGCCCGCGCTGCTGGCGCTGACGGCCCGGGAGTC |
| | | GTCGGTGCGGTCGGGCGGCGCGGGCGGCGCGGGCGCCCCGGGGGCCGT |
| | | GGTGGTGGACGTGGACCTGACGCCCGCGGCACCCAGCAGCGAGTCGCT |
| | | GGCCCTGGACGAAGTGACAGCCATGGACAACCACGTGGCAGGGCTCGG |
| | | GCCCGCGGAGGAGCGGCGTGCGCTGGTGGGTCCCGGCTCTCCGCCCCG |
| | | CAGCGCGCCCGGCCAGCTCCCATCGCCCCGGGCGCACAGCCTCAACCC |
| | | CGACGCCTCGGGCTCCAGCTGCAGCCTGGCCCGGACGCGCTCCCGAGA |
| | | AAGCTGCGCCAGCGTGCGCCGCGCCTCGTCGGCCGACGACATCGAGGC |
| | | CATGCGCGCCGGGGTGCTGCCCCCGCCACCGCGCCACGCCAGCACCGG |
| | | GGCCATGCACCCACTGCGCAGCGGCTTGCTCAACTCCACCTCGGACTCC |
| | | GACCTCGTGCGCTACCGCACCATTAGCAAGATTCCCCAAATCACCCTCA |
| | | ACTTTGTGGACCTCAAGGGCGACCCCTTCTTGGCTTCGCCCACCAGTGA |
| | | CCGTGAGATCATAGCACCTAAGATAAAGGAGCGAACCCACAATGTCAC |
| | | TGAGAAGGTCACCCAGGTCCTGTCCCTGGGCGCCGACGTGCTGCCTGAG |
| | | TACAAGCTGCAGGCACCGCGCATCCACCGCTGGACCATCCTGCATTACA |
| | | GCCCCTTCAAGGCCGTGTGGGACTGGCTCATCCTGCTGCTGGTCATCTA |
| | | CACGGCTGTCTTCACACCCTACTCGGCTGCCTTCCTGCTGAAGGAGACG |
| | | GAAGAAGGCCCGCCTGCTACCGAGTGTGGCTACGCCTGCCAGCCGCTG |
| | | GCTGTGGTGGACCTCATCGTGGACATCATGTTCATTGTGGACATCCTCA |
| | | TCAACTTCCGCACCACCTACGTCAATGCCAACGAGGAGGTGGTCAGCC |
| | | ACCCCGGCCGCATCGCCGTCCACTACTTCAAGGGCTGGTTCCTCATCGA |
| | | CATGGTGGCCGCCATCCCCTTCGACCTGCTCATCTTCGGCTCTGGCTCTG |
| | | AGGAGCTGATCGGGCTGCTGAAGACTGCGCGGCTGCTGCGGCTGGTGC |
| | | GCGTGGCGCGGAAGCTGGATCGCTACTCAGAGTACGGCGCGGCCGTGC |
| | | TGTTCTTGCTCATGTGCACCTTTGCGCTCATCGCGCACTGGCTAGCCTGC |
| | | ATCTGGTACGCCATCGGCAACATGGAGCAGCCACACATGGACTCACGC |
| | | ATCGGCTGGCTGCACAACCTGGGCGACCAGATAGGCAAACCCTACAAC |
| | | AGCAGCGGCCTGGGCGGCCCCTCCATCAAGGACAAGTATGTGACGGCG |
| | | CTCTACTTCACCTTCAGCAGCCTCACCAGTGTGGGCTTCGGCAACGTCT |
| | | CTCCCAACACCAACTCAGAGAAGATCTTCTCCATCTGCGTCATGCTCAT |
| | | TGGCTCCCTCATGTATGCTAGCATCTTCGGCAACGTGTCGGCCATCATC |
| | | CAGCGGCTGTACTCGGGCACAGCCCGCTACCACACACAGATGCTGCGG |
| | | GTGCGGGAGTTCATCCGCTTCCACCAGATCCCCAATCCCCTGCGCCAGC |
| | | GCCTCGAGGAGTACTTCCAGCACGCCTGGTCCTACACCAACGGCATCGA |
| | | CATGAACGCGGTGCTGAAGGGCTTCCCTGAGTGCCTGCAGGCTGACATC |
| | | TGCCTGCACCTGAACCGCTCACTGCTGCAGCACTGCAAACCCTTCCGAG |
| | | GGGCCACCAAGGGCTGCCTTCGGGCCCTGGCCATGAAGTTCAAGACCA |
| | | CACATGCACCGCCAGGGGACACACTGGTGCATGCTGGGGACCTGCTCA |
| | | CCGCCCTGTACTTCATCTCCCGGGGCTCCATCGAGATCCTGCGGGGCGA |
| | | CGTCGTCGTGGCCATCCTGGGGAAGAATGACATCTTTGGGGAGCCTCTG |
| | | AACCTGTATGCAAGGCCTGGCAAGTCGAACGGGGATGTGCGGGCCCTC |
| | | ACCTACTGTGACCTACACAAGATCCATCGGGACGACCTGCTGGAGGTG |
| | | CTGGACATGTACCCTGAGTTCTCCGACCACTTCTGGTCCAGCCTGGAGA |
| | | TCACCTTCAACCTGCGAGATACCAACATGATCCCGGGCTCCCCCGGCAG |
| | | TACGGAGTTAGAGGGTGGCTTCAGTCGGCAACGCAAGCGCAAGTTGTC |
| | | CTTCCGCAGGCGCACGGACAAGGACACGGAGCAGCCAGGGGAGGTGTC |
| | | GGCCTTGGGGCCGGGCCGGGCGGGGGCAGGGCCGAGTAGCCGGGGCTA |
| | | G |
| 2 | G628S KCNH2 | ATGCCGGTGCGGAGGGGCCACGTCGCGCCGCAGAACACCTTCCTGGAC |
| | (truncated) | ACCATCATCCGCAAGTTTGAGGGCCAGAGCCGTAAGTTCATCATCGCCA |
| | | ACGCTCGGGTGGAGAACTGCGCCGTCATCTACTGCAACGACGGCTTCTG |
| | | CGAGCTGTGCGGCTACTCGCGGGCCGAGGTGATGCAGCGACCCTGCAC |
| | | CTGCGACTTCCTGCACGGGCCGCGCACGCAGCGCCGCGCTGCCGCGCA |
| | | GATCGCGCAGGCACTGCTGGGCGCCGAGGAGCGCAAAGTGGAAATCGC |
| | | CTTCTACCGGAAAGATGGGAGCTGCTTCCTATGTCTGGTGGATGTGGTG |
| | | CCCGTGAAGAACGAGGATGGGGCTGTCATCATGTTCATCCTCAATTTCG |
| | | AGGTGGTGATGGAGAAGGACATGGTGGGGTCCCCGGCTCATGACACCA |
| | | ACCACCGGGGCCCCCCCACCAGCTGGCTGGCCCCAGGCCGCGCCAAGA |
| | | CCTTCCGCCTGAAGCTGCCCGCGCTGCTGGCGCTGACGGCCCGGGAGTC |
| | | GTCGGTGCGGTCGGGCGGCGCGGGCGGCGCGGGCGCCCCGGGGGCCGT |
| | | GGTGGTGGACGTGGACCTGACGCCCGCGGCACCCAGCAGCGAGTCGCT |
| | | GGCCCTGGACGAAGTGACAGCCATGGACAACCACGTGGCAGGGCTCGG |
| | | GCCCGCGGAGGAGCGGCGTGCGCTGGTGGGTCCCGGCTCTCCGCCCCG |
| | | CAGCGCGCCCGGCCAGCTCCCATCGCCCCGGGCGCACAGCCTCAACCC |
| | | CGACGCCTCGGGCTCCAGCTGCAGCCTGGCCCGGACGCGCTCCCGAGA |
| | | AAGCTGCGCCAGCGTGCGCCGCGCCTCGTCGGCCGACGACATCGAGGC |
| | | CATGCGCGCCGGGGTGCTGCCCCCGCCACCGCGCCACGCCAGCACCGG |
| | | GGCCATGCACCCACTGCGCAGCGGCTTGCTCAACTCCACCTCGGACTCC |
| | | GACCTCGTGCGCTACCGCACCATTAGCAAGATTCCCCAAATCACCCTCA |
| | | ACTTTGTGGACCTCAAGGGCGACCCCTTCTTGGCTTCGCCCACCAGTGA |
| | | CCGTGAGATCATAGCACCTAAGATAAAGGAGCGAACCCACAATGTCAC |
| | | TGAGAAGGTCACCCAGGTCCTGTCCCTGGGCGCCGACGTGCTGCCTGAG |
| | | TACAAGCTGCAGGCACCGCGCATCCACCGCTGGACCATCCTGCATTACA |
| | | GCCCCTTCAAGGCCGTGTGGGACTGGCTCATCCTGCTGCTGGTCATCTA |
| | | CACGGCTGTCTTCACACCCTACTCGGCTGCCTTCCTGCTGAAGGAGACG |
| | | GAAGAAGGCCCGCCTGCTACCGAGTGTGGCTACGCCTGCCAGCCGCTG |
| | | GCTGTGGTGGACCTCATCGTGGACATCATGTTCATTGTGGACATCCTCA |
| | | TCAACTTCCGCACCACCTACGTCAATGCCAACGAGGAGGTGGTCAGCC |
| | | ACCCCGGCCGCATCGCCGTCCACTACTTCAAGGGCTGGTTCCTCATCGA |
| | | CATGGTGGCCGCCATCCCCTTCGACCTGCTCATCTTCGGCTCTGGCTCTG |
| | | AGGAGCTGATCGGGCTGCTGAAGACTGCGCGGCTGCTGCGGCTGGTGC |
| | | GCGTGGCGCGGAAGCTGGATCGCTACTCAGAGTACGGCGCGGCCGTGC |
| | | TGTTCTTGCTCATGTGCACCTTTGCGCTCATCGCGCACTGGCTAGCCTGC |
| | | ATCTGGTACGCCATCGGCAACATGGAGCAGCCACACATGGACTCACGC |
| | | ATCGGCTGGCTGCACAACCTGGGCGACCAGATAGGCAAACCCTACAAC |
| | | AGCAGCGGCCTGGGCGGCCCCTCCATCAAGGACAAGTATGTGACGGCG |
| | | CTCTACTTCACCTTCAGCAGCCTCACCAGTGTGGGCTTCTCCAACGTCTC |
| | | TCCCAACACCAACTCAGAGAAGATCTTCTCCATCTGCGTCATGCTCATT |
| | | GGCTCCCTCATGTATGCTAGCATCTTCGGCAACGTGTCGGCCATCATCC |
| | | AGCGGCTGTACTCGGGCACAGCCCGCTACCACACACAGATGCTGCGGG |
| | | TGCGGGAGTTCATCCGCTTCCACCAGATCCCCAATCCCCTGCGCCAGCG |
| | | CCTCGAGGAGTACTTCCAGCACGCCTGGTCCTACACCAACGGCATCGAC |
| | | ATGAACGCGGTGCTGAAGGGCTTCCCTGAGTGCCTGCAGGCTGACATCT |
| | | GCCTGCACCTGAACCGCTCACTGCTGCAGCACTGCAAACCCTTCCGAGG |
| | | GGCCACCAAGGGCTGCCTTCGGGCCCTGGCCATGAAGTTCAAGACCAC |
| | | ACATGCACCGCCAGGGGACACACTGGTGCATGCTGGGGACCTGCTCAC |
| | | CGCCCTGTACTTCATCTCCCGGGGCTCCATCGAGATCCTGCGGGGCGAC |
| | | GTCGTCGTGGCCATCCTGGGGAAGAATGACATCTTTGGGGAGCCTCTGA |
| | | ACCTGTATGCAAGGCCTGGCAAGTCGAACGGGGATGTGCGGGCCCTCA |
| | | CCTACTGTGACCTACACAAGATCCATCGGGACGACCTGCTGGAGGTGCT |
| | | GGACATGTACCCTGAGTTCTCCGACCACTTCTGGTCCAGCCTGGAGATC |
| | | ACCTTCAACCTGCGAGATACCAACATGATCCCGGGCTCCCCCGGCAGTA |
| | | CGGAGTTAGAGGGTGGCTTCAGTCGGCAACGCAAGCGCAAGTTGTCCT |
| | | TCCGCAGGCGCACGGACAAGGACACGGAGCAGCCAGGGGAGGTGTCG |
| | | GCCTTGGGGCCGGGCCGGGCGGGGGCAGGGCCGAGTAGCCGGGGCTAG |
| 3 | Anf1 promoter | CCCCCCCTCAATCCCTTACCTCCTCCTTCTATTTCCCTGGGAAGGGTGGG |
| | (mouse) | ACCACCACATATTTCATGCTCAGAGTCTGGGCTTTGGAAAGAAGAAAA |
| | | GTGGTTTCATCCTCCAAACCCATCCTGTTGGCACCTTGGACACGAGTCT |
| | | TGGGAGGCAGTAAAAATAAACTTTAGAGGAAATATGCTCTTCTAACAT |
| | | CCCTTGGTGTTTACACGTGTAGCTGAATTCTTTAGAGCCTGTATCATGTT |
| | | GGCTTCCTGGCTGACTTCATACTCTAAAAAAAAAATAATAGCTCTTTCA |
| | | CCTGACTGCTAACAGGGACATCTAGGGTGGGGGTGGGCTGTCTGGGGC |
| | | CAGAGGTCCACCCACGAGGCCGATGAATCAGGTGTGAAGCTAGCTCCA |
| | | GCATGTGTACTCCCTGGCCAGCCTAGCTGGCCTCCCAGCTGCCTGTCAT |
| | | TGCCTCCTCTCCCGCCCTTATTTGGAGCCCCTGACAGCTGAGCAGCAAG |
| | | CAGGGGGAGCTGGGTGCAGGCCAGCCGTCACCCTCTGCTTCCCTGCATG |
| | | GGTCCTGTTGCCAGGGAGAAAGAATCCTGAGGCGAGCGCCCAGGAAGA |
| | | TAACCAAGGACTCTTTTCTGCTCCTCTCACACCTTTGAAGTGGGGGCCT |
| | | CTTGAGGCAAATCAGCAAGAATGTGACTCTTGCAGCTGAGGGTCTGGG |
| | | GGAGGGGGGTGAGTGGAGCTGCTCAAGGCAAAGGGGCCGTGACAAGC |
| | | TTTGCCGAACTGATAACTTTAAAAGGGCATCTTCTGCTGGCTCCTCACT |
| | | CCATCGCTTTATCGCTGCAAGTGACAGAATGGGGAGGGTTCTAGCCCCC |
| | | CTGCCTTCTCAAAGAGCTGGGGGGCTATAAAAACGGGAGATGCTGGCA |
| | | GCTAGGAGACAGTGACGGACAAAAGCTGAGAGAGAGAGAGAAAGAAA |
| | | CCAGAGTGGGCAGAGACAGCAAACATCAGATCGTGCCCCGACCCACGC |
| | | CAGC |
| 4 | CMV enhancer | CGTTACATAACTTACGGTAAATGGCCCGCCTGGCTGACCGCCCAACGAC |
| | | CCCCGCCCATTGACGTCAATAATGACGTATGTTCCCATAGTAACGCCAA |
| | | TAGGGACTTTCCATTGACGTCAATGGGTGGAGTATTTACGGTAAACTGC |
| | | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATG |
| 5 | CMV-e/Anf1-p | CGTTACATAACTTACGGTAAATGGCCCGCCTGGCTGACCGCCCAACGAC |
| | (mouse) hybrid | CCCCGCCCATTGACGTCAATAATGACGTATGTTCCCATAGTAACGCCAA |
| | regulatory | TAGGGACTTTCCATTGACGTCAATGGGTGGAGTATTTACGGTAAACTGC |
| | element | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATGCCCCCCCTCAATCCCTTACCTCCTCCTTCTATTTCCCTGG |
| | | GAAGGGTGGGACCACCACATATTTCATGCTCAGAGTCTGGGCTTTGGAA |
| | | AGAAGAAAAGTGGTTTCATCCTCCAAACCCATCCTGTTGGCACCTTGGA |
| | | CACGAGTCTTGGGAGGCAGTAAAAATAAACTTTAGAGGAAATATGCTC |
| | | TTCTAACATCCCTTGGTGTTTACACGTGTAGCTGAATTCTTTAGAGCCTG |
| | | TATCATGTTGGCTTCCTGGCTGACTTCATACTCTAAAAAAAAAATAATA |
| | | GCTCTTTCACCTGACTGCTAACAGGGACATCTAGGGTGGGGGTGGGCTG |
| | | TCTGGGGCCAGAGGTCCACCCACGAGGCCGATGAATCAGGTGTGAAGC |
| | | TAGCTCCAGCATGTGTACTCCCTGGCCAGCCTAGCTGGCCTCCCAGCTG |
| | | CCTGTCATTGCCTCCTCTCCCGCCCTTATTTGGAGCCCCTGACAGCTGAG |
| | | CAGCAAGCAGGGGGAGCTGGGTGCAGGCCAGCCGTCACCCTCTGCTTC |
| | | CCTGCATGGGTCCTGTTGCCAGGGAGAAAGAATCCTGAGGCGAGCGCC |
| | | CAGGAAGATAACCAAGGACTCTTTTCTGCTCCTCTCACACCTTTGAAGT |
| | | GGGGGCCTCTTGAGGCAAATCAGCAAGAATGTGACTCTTGCAGCTGAG |
| | | GGTCTGGGGGAGGGGGGTGAGTGGAGCTGCTCAAGGCAAAGGGGCCGT |
| | | GACAAGCTTTGCCGAACTGATAACTTTAAAAGGGCATCTTCTGCTGGCT |
| | | CCTCACTCCATCGCTTTATCGCTGCAAGTGACAGAATGGGGAGGGTTCT |
| | | AGCCCCCCTGCCTTCTCAAAGAGCTGGGGGGCTATAAAAACGGGAGAT |
| | | GCTGGCAGCTAGGAGACAGTGACGGACAAAAGCTGAGAGAGAGAGAG |
| | | AAAGAAACCAGAGTGGGCAGAGACAGCAAACATCAGATCGTGCCCCG |
| | | ACCCACGCCAGC |
| 6 | Anf1 enhancer | CTCAGCCGTTCTTCAAGTCTTTGCCCAACTCAAAGACCATCTCCAGGAG |
| | (mouse) | GTAGGTCATGTGACACACACAGCAAGGAGCCCGAGTTCCCCCAGTAGG |
| | | GCTGCTCGCCTAGTCCTCTCTGCTAATAATAATCCCCAGGCACACAATT |
| | | AAGCAGTGAGCGCATCCCAGGGACTCATCCGGAGGCGACAGTGTGAAC |
| | | TTGCACAGATTTCAGACTTCAAGGAGAGATTGGCCAACCCAAATTGGTT |
| | | GCCTACAACCCCCAGCTTTCTCAAAGGGCTCTCTTTTTCTATCTTAGAGA |
| | | AAACATCACCGCAGACACCTCTTGTGACTTCCAGCCACATGACAACTCC |
| | | ACCAGGCAGCCACAGGGTCAATGGGCGTGGAAGTCTTCCAATAGCCCA |
| | | TTAAGACGGTCAGAAAGCAGAGAGCAGCAGAAAATGCACACACTTATC |
| | | ACACACACAAAGCATACATATGTCGAAAATGGCTACACTGGGGTGTGG |
| | | AGACCACTTGTTAAAGGTGGACTGCCTGCTCATGCTCCTGAGGCTGCCA |
| | | GAAGAATTCATGGCTCACTTCCTTGGTCTCAAAGGTCATGAAGGACATT |
| | | ATGGAGAATGGTGTATGTGT |
| 7 | CMV promoter | GTGATGCGGTTTTGGCAGTACATCAATGGGCGTGGATAGCGGTTTGACT |
| | | CACGGGGATTTCCAAGTCTCCACCCCATTGACGTCAATGGGAGTTTGTT |
| | | TTGGCACCAAAATCAACGGGACTTTCCAAAATGTCGTAACAACTCCGCC |
| | | CCATTGACGCAAATGGGCGGTAGGCGTGTACGGTGGGAGGTCTATATA |
| | | AGCAGAGCTCATCGCTATTACC |
| 8 | Anf1 promoter | CCTCCTTCACCCCCACCTCCCTTCCTCCATCGGTCAAGTTGCCACGGGCA |
| | (human) | CAAGCCCAGGAAGGGCAGGGGGATGTTAACAAACTTCAGACACGAAG |
| | | AGTCTGAGCATCTATAAACAGCAACGGAAGAAATGAAATTGGCTGCGT |
| | | CCTCTAAGCCTGTCCCCGCAGCATGCTGGAGGAGGGTCGCGGGGGACA |
| | | TGGAAGAGGAGGAGCTTTGGAGAGAGGATGCTTGTGCTCCCCCGCCTTT |
| | | TCTTGCTATTTCTATTTGGGGGTTGGATTCCTGGGAGCTTCATCACATTT |
| | | GGTTCTCAGCTGACTTTATATACTAAAAAATAACTTCCTTTCGCCTGACC |
| | | ATGGAGAGGGACTGCCAGGGGTGAAGGCAGCCCTGTCTGAGGCCAGAG |
| | | GTCTGCCCACGTGGCGGATGAGGCAGGTGTGAGGCCAGCTTGAGCATC |
| | | TGGATCCATTTGTCTCGGGCTGCTGGCTGCCTGCCATTTCCTCCTCTCCA |
| | | CCCTTATTTGGAGGCCCTGACAGCTGAGCCACAAACAAACCAGGGGAG |
| | | CTGGGCACCAGCCAAGCGTCACCCTCTGTTTCCCCGCACGGGTACCAGC |
| | | GTCGAGGAGAAAGAATCCTGAGGCACGGCGGTGAGATAACCAAGGACT |
| | | CTTTTTTACTCTTCTCACACCTTTGAAGTGGGAGCCTCTTGAGTCAAATC |
| | | AGTAAGAATGCGGCTCTTGCAGCTGAGGGTCTGGGGGGCTGTTGGGGC |
| | | TGCCCAAGGCAGAGAGGGGCTGTGACAAGCCCTGCGGGATGATAACTT |
| | | TAAAAGGGCATCTCCTGCTGGCTTCTCACTTGGCAGCTTTATCACTGCA |
| | | AGTGACAGAATGGGGAGGGTTCTGTCTCTCCTGCGTGCTTGGAGAGCTG |
| | | GGGGGCTATAAAAAGAGGCGGCACTGGGCAGCTGGGAGACAGGGACA |
| | | GACGTAGGCCAAGAGAGGGGAACCAGAGAGGAACCAGAGGGGAGAGA |
| | | CAGAGCAGCAAGCAGTGGATTGCTCCTTGACGACGCCAG |
| 9 | CMV-e/Anf1-p | CGTTACATAACTTACGGTAAATGGCCCGCCTGGCTGACCGCCCAACGAC |
| | (human) hybrid | CCCCGCCCATTGACGTCAATAATGACGTATGTTCCCATAGTAACGCCAA |
| | regulatory | TAGGGACTTTCCATTGACGTCAATGGGTGGAGTATTTACGGTAAACTGC |
| | element | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATGCCTCCTTCACCCCCACCTCCCTTCCTCCATCGGTCAAGTT |
| | | GCCACGGGCACAAGCCCAGGAAGGGCAGGGGGATGTTAACAAACTTCA |
| | | GACACGAAGAGTCTGAGCATCTATAAACAGCAACGGAAGAAATGAAAT |
| | | TGGCTGCGTCCTCTAAGCCTGTCCCCGCAGCATGCTGGAGGAGGGTCGC |
| | | GGGGGACATGGAAGAGGAGGAGCTTTGGAGAGAGGATGCTTGTGCTCC |
| | | CCCGCCTTTTCTTGCTATTTCTATTTGGGGGTTGGATTCCTGGGAGCTTC |
| | | ATCACATTTGGTTCTCAGCTGACTTTATATACTAAAAAATAACTTCCTTT |
| | | CGCCTGACCATGGAGAGGGACTGCCAGGGGTGAAGGCAGCCCTGTCTG |
| | | AGGCCAGAGGTCTGCCCACGTGGCGGATGAGGCAGGTGTGAGGCCAGC |
| | | TTGAGCATCTGGATCCATTTGTCTCGGGCTGCTGGCTGCCTGCCATTTCC |
| | | TCCTCTCCACCCTTATTTGGAGGCCCTGACAGCTGAGCCACAAACAAAC |
| | | CAGGGGAGCTGGGCACCAGCCAAGCGTCACCCTCTGTTTCCCCGCACG |
| | | GGTACCAGCGTCGAGGAGAAAGAATCCTGAGGCACGGCGGTGAGATAA |
| | | CCAAGGACTCTTTTTTACTCTTCTCACACCTTTGAAGTGGGAGCCTCTTG |
| | | AGTCAAATCAGTAAGAATGCGGCTCTTGCAGCTGAGGGTCTGGGGGGC |
| | | TGTTGGGGCTGCCCAAGGCAGAGAGGGGCTGTGACAAGCCCTGCGGGA |
| | | TGATAACTTTAAAAGGGCATCTCCTGCTGGCTTCTCACTTGGCAGCTTT |
| | | ATCACTGCAAGTGACAGAATGGGGAGGGTTCTGTCTCTCCTGCGTGCTT |
| | | GGAGAGCTGGGGGGCTATAAAAAGAGGCGGCACTGGGCAGCTGGGAG |
| | | ACAGGGACAGACGTAGGCCAAGAGAGGGGAACCAGAGAGGAACCAGA |
| | | GGGGAGAGACAGAGCAGCAAGCAGTGGATTGCTCCTTGACGACGCCAG |
| 10 | Bridging primer | CTAGTAATGGGGCTGCCCGGTAC |
| | (forward) | |
| 11 | Bridging primer | GATCATTACCCCGACGGGC |
| | (reverse) | |
| 12 | Wild-type | ATGCCGGTGCGGAGGGGCCACGTCGCGCCGCAGAACACCTTCCTGGAC |
| | KCNH2 (full- | ACCATCATCCGCAAGTTTGAGGGCCAGAGCCGTAAGTTCATCATCGCCA |
| | length) | ACGCTCGGGTGGAGAACTGCGCCGTCATCTACTGCAACGACGGCTTCTG |
| | | CGAGCTGTGCGGCTACTCGCGGGCCGAGGTGATGCAGCGACCCTGCAC |
| | | CTGCGACTTCCTGCACGGGCCGCGCACGCAGCGCCGCGCTGCCGCGCA |
| | | GATCGCGCAGGCACTGCTGGGCGCCGAGGAGCGCAAAGTGGAAATCGC |
| | | CTTCTACCGGAAAGATGGGAGCTGCTTCCTATGTCTGGTGGATGTGGTG |
| | | CCCGTGAAGAACGAGGATGGGGCTGTCATCATGTTCATCCTCAATTTCG |
| | | AGGTGGTGATGGAGAAGGACATGGTGGGGTCCCCGGCTCATGACACCA |
| | | ACCACCGGGGCCCCCCCACCAGCTGGCTGGCCCCAGGCCGCGCCAAGA |
| | | CCTTCCGCCTGAAGCTGCCCGCGCTGCTGGCGCTGACGGCCCGGGAGTC |
| | | GTCGGTGCGGTCGGGCGGCGCGGGCGGCGCGGGCGCCCCGGGGGCCGT |
| | | GGTGGTGGACGTGGACCTGACGCCCGCGGCACCCAGCAGCGAGTCGCT |
| | | GGCCCTGGACGAAGTGACAGCCATGGACAACCACGTGGCAGGGCTCGG |
| | | GCCCGCGGAGGAGCGGCGTGCGCTGGTGGGTCCCGGCTCTCCGCCCCG |
| | | CAGCGCGCCCGGCCAGCTCCCATCGCCCCGGGCGCACAGCCTCAACCC |
| | | CGACGCCTCGGGCTCCAGCTGCAGCCTGGCCCGGACGCGCTCCCGAGA |
| | | AAGCTGCGCCAGCGTGCGCCGCGCCTCGTCGGCCGACGACATCGAGGC |
| | | CATGCGCGCCGGGGTGCTGCCCCCGCCACCGCGCCACGCCAGCACCGG |
| | | GGCCATGCACCCACTGCGCAGCGGCTTGCTCAACTCCACCTCGGACTCC |
| | | GACCTCGTGCGCTACCGCACCATTAGCAAGATTCCCCAAATCACCCTCA |
| | | ACTTTGTGGACCTCAAGGGCGACCCCTTCTTGGCTTCGCCCACCAGTGA |
| | | CCGTGAGATCATAGCACCTAAGATAAAGGAGCGAACCCACAATGTCAC |
| | | TGAGAAGGTCACCCAGGTCCTGTCCCTGGGCGCCGACGTGCTGCCTGAG |
| | | TACAAGCTGCAGGCACCGCGCATCCACCGCTGGACCATCCTGCATTACA |
| | | GCCCCTTCAAGGCCGTGTGGGACTGGCTCATCCTGCTGCTGGTCATCTA |
| | | CACGGCTGTCTTCACACCCTACTCGGCTGCCTTCCTGCTGAAGGAGACG |
| | | GAAGAAGGCCCGCCTGCTACCGAGTGTGGCTACGCCTGCCAGCCGCTG |
| | | GCTGTGGTGGACCTCATCGTGGACATCATGTTCATTGTGGACATCCTCA |
| | | TCAACTTCCGCACCACCTACGTCAATGCCAACGAGGAGGTGGTCAGCC |
| | | ACCCCGGCCGCATCGCCGTCCACTACTTCAAGGGCTGGTTCCTCATCGA |
| | | CATGGTGGCCGCCATCCCCTTCGACCTGCTCATCTTCGGCTCTGGCTCTG |
| | | AGGAGCTGATCGGGCTGCTGAAGACTGCGCGGCTGCTGCGGCTGGTGC |
| | | GCGTGGCGCGGAAGCTGGATCGCTACTCAGAGTACGGCGCGGCCGTGC |
| | | TGTTCTTGCTCATGTGCACCTTTGCGCTCATCGCGCACTGGCTAGCCTGC |
| | | ATCTGGTACGCCATCGGCAACATGGAGCAGCCACACATGGACTCACGC |
| | | ATCGGCTGGCTGCACAACCTGGGCGACCAGATAGGCAAACCCTACAAC |
| | | AGCAGCGGCCTGGGCGGCCCCTCCATCAAGGACAAGTATGTGACGGCG |
| | | CTCTACTTCACCTTCAGCAGCCTCACCAGTGTGGGCTTCGGCAACGTCT |
| | | CTCCCAACACCAACTCAGAGAAGATCTTCTCCATCTGCGTCATGCTCAT |
| | | TGGCTCCCTCATGTATGCTAGCATCTTCGGCAACGTGTCGGCCATCATC |
| | | CAGCGGCTGTACTCGGGCACAGCCCGCTACCACACACAGATGCTGCGG |
| | | GTGCGGGAGTTCATCCGCTTCCACCAGATCCCCAATCCCCTGCGCCAGC |
| | | GCCTCGAGGAGTACTTCCAGCACGCCTGGTCCTACACCAACGGCATCGA |
| | | CATGAACGCGGTGCTGAAGGGCTTCCCTGAGTGCCTGCAGGCTGACATC |
| | | TGCCTGCACCTGAACCGCTCACTGCTGCAGCACTGCAAACCCTTCCGAG |
| | | GGGCCACCAAGGGCTGCCTTCGGGCCCTGGCCATGAAGTTCAAGACCA |
| | | CACATGCACCGCCAGGGGACACACTGGTGCATGCTGGGGACCTGCTCA |
| | | CCGCCCTGTACTTCATCTCCCGGGGCTCCATCGAGATCCTGCGGGGCGA |
| | | CGTCGTCGTGGCCATCCTGGGGAAGAATGACATCTTTGGGGAGCCTCTG |
| | | AACCTGTATGCAAGGCCTGGCAAGTCGAACGGGGATGTGCGGGCCCTC |
| | | ACCTACTGTGACCTACACAAGATCCATCGGGACGACCTGCTGGAGGTG |
| | | CTGGACATGTACCCTGAGTTCTCCGACCACTTCTGGTCCAGCCTGGAGA |
| | | TCACCTTCAACCTGCGAGATACCAACATGATCCCGGGCTCCCCCGGCAG |
| | | TACGGAGTTAGAGGGTGGCTTCAGTCGGCAACGCAAGCGCAAGTTGTC |
| | | CTTCCGCAGGCGCACGGACAAGGACACGGAGCAGCCAGGGGAGGTGTC |
| | | GGCCTTGGGGCCGGGCCGGGCGGGGGCAGGGCCGAGTAGCCGGGGCC |
| | | GGCCGGGGGGGCCGTGGGGGGAGAGCCCGTCCAGTGGCCCCTCCAGCC |
| | | CTGAGAGCAGTGAGGATGAGGGCCCAGGCCGCAGCTCCAGCCCCCTCC |
| | | GCCTGGTGCCCTTCTCCAGCCCCAGGCCCCCCGGAGAGCCGCCGGGTGG |
| | | GGAGCCCCTGATGGAGGACTGCGAGAAGAGCAGCGACACTTGCAACCC |
| | | CCTGTCAGGCGCCTTCTCAGGAGTGTCCAACATTTTCAGCTTCTGGGGG |
| | | GACAGTCGGGGCCGCCAGTACCAGGAGCTCCCTCGATGCCCCGCCCCC |
| | | ACCCCCAGCCTCCTCAACATCCCCCTCTCCAGCCCGGGTCGGCGGCCCC |
| | | GGGGCGACGTGGAGAGCAGGCTGGATGCCCTCCAGCGCCAGCTCAACA |
| | | GGCTGGAGACCCGGCTGAGTGCAGACATGGCCACTGTCCTGCAGCTGC |
| | | TACAGAGGCAGATGACGCTGGTCCCGCCCGCCTACAGTGCTGTGACCA |
| | | CCCCGGGGCCTGGCCCCACTTCCACATCCCCGCTGTTGCCCGTCAGCCC |
| | | CCTCCCCACCCTCACCTTGGACTCGCTTTCTCAGGTTTCCCAGTTCATGG |
| | | CGTGTGAGGAGCTGCCCCCGGGGGCCCCAGAGCTTCCCCAAGAAGGCC |
| | | CCACACGACGCCTCTCCCTACCGGGCCAGCTGGGGGCCCTCACCTCCCA |
| | | GCCCCTGCACAGACACGGCTCGGACCCGGGCAGTTAG |
| 13 | G628S KCNH2 | ATGCCGGTGCGGAGGGGCCACGTCGCGCCGCAGAACACCTTCCTGGAC |
| | (full-length) | ACCATCATCCGCAAGTTTGAGGGCCAGAGCCGTAAGTTCATCATCGCCA |
| | | ACGCTCGGGTGGAGAACTGCGCCGTCATCTACTGCAACGACGGCTTCTG |
| | | CGAGCTGTGCGGCTACTCGCGGGCCGAGGTGATGCAGCGACCCTGCAC |
| | | CTGCGACTTCCTGCACGGGCCGCGCACGCAGCGCCGCGCTGCCGCGCA |
| | | GATCGCGCAGGCACTGCTGGGCGCCGAGGAGCGCAAAGTGGAAATCGC |
| | | CTTCTACCGGAAAGATGGGAGCTGCTTCCTATGTCTGGTGGATGTGGTG |
| | | CCCGTGAAGAACGAGGATGGGGCTGTCATCATGTTCATCCTCAATTTCG |
| | | AGGTGGTGATGGAGAAGGACATGGTGGGGTCCCCGGCTCATGACACCA |
| | | ACCACCGGGGCCCCCCCACCAGCTGGCTGGCCCCAGGCCGCGCCAAGA |
| | | CCTTCCGCCTGAAGCTGCCCGCGCTGCTGGCGCTGACGGCCCGGGAGTC |
| | | GTCGGTGCGGTCGGGCGGCGCGGGCGGCGCGGGCGCCCCGGGGGCCGT |
| | | GGTGGTGGACGTGGACCTGACGCCCGCGGCACCCAGCAGCGAGTCGCT |
| | | GGCCCTGGACGAAGTGACAGCCATGGACAACCACGTGGCAGGGCTCGG |
| | | GCCCGCGGAGGAGCGGCGTGCGCTGGTGGGTCCCGGCTCTCCGCCCCG |
| | | CAGCGCGCCCGGCCAGCTCCCATCGCCCCGGGCGCACAGCCTCAACCC |
| | | CGACGCCTCGGGCTCCAGCTGCAGCCTGGCCCGGACGCGCTCCCGAGA |
| | | AAGCTGCGCCAGCGTGCGCCGCGCCTCGTCGGCCGACGACATCGAGGC |
| | | CATGCGCGCCGGGGTGCTGCCCCCGCCACCGCGCCACGCCAGCACCGG |
| | | GGCCATGCACCCACTGCGCAGCGGCTTGCTCAACTCCACCTCGGACTCC |
| | | GACCTCGTGCGCTACCGCACCATTAGCAAGATTCCCCAAATCACCCTCA |
| | | ACTTTGTGGACCTCAAGGGCGACCCCTTCTTGGCTTCGCCCACCAGTGA |
| | | CCGTGAGATCATAGCACCTAAGATAAAGGAGCGAACCCACAATGTCAC |
| | | TGAGAAGGTCACCCAGGTCCTGTCCCTGGGCGCCGACGTGCTGCCTGAG |
| | | TACAAGCTGCAGGCACCGCGCATCCACCGCTGGACCATCCTGCATTACA |
| | | GCCCCTTCAAGGCCGTGTGGGACTGGCTCATCCTGCTGCTGGTCATCTA |
| | | CACGGCTGTCTTCACACCCTACTCGGCTGCCTTCCTGCTGAAGGAGACG |
| | | GAAGAAGGCCCGCCTGCTACCGAGTGTGGCTACGCCTGCCAGCCGCTG |
| | | GCTGTGGTGGACCTCATCGTGGACATCATGTTCATTGTGGACATCCTCA |
| | | TCAACTTCCGCACCACCTACGTCAATGCCAACGAGGAGGTGGTCAGCC |
| | | ACCCCGGCCGCATCGCCGTCCACTACTTCAAGGGCTGGTTCCTCATCGA |
| | | CATGGTGGCCGCCATCCCCTTCGACCTGCTCATCTTCGGCTCTGGCTCTG |
| | | AGGAGCTGATCGGGCTGCTGAAGACTGCGCGGCTGCTGCGGCTGGTGC |
| | | GCGTGGCGCGGAAGCTGGATCGCTACTCAGAGTACGGCGCGGCCGTGC |
| | | TGTTCTTGCTCATGTGCACCTTTGCGCTCATCGCGCACTGGCTAGCCTGC |
| | | ATCTGGTACGCCATCGGCAACATGGAGCAGCCACACATGGACTCACGC |
| | | ATCGGCTGGCTGCACAACCTGGGCGACCAGATAGGCAAACCCTACAAC |
| | | AGCAGCGGCCTGGGCGGCCCCTCCATCAAGGACAAGTATGTGACGGCG |
| | | CTCTACTTCACCTTCAGCAGCCTCACCAGTGTGGGCTTCTCCAACGTCTC |
| | | TCCCAACACCAACTCAGAGAAGATCTTCTCCATCTGCGTCATGCTCATT |
| | | GGCTCCCTCATGTATGCTAGCATCTTCGGCAACGTGTCGGCCATCATCC |
| | | AGCGGCTGTACTCGGGCACAGCCCGCTACCACACACAGATGCTGCGGG |
| | | TGCGGGAGTTCATCCGCTTCCACCAGATCCCCAATCCCCTGCGCCAGCG |
| | | CCTCGAGGAGTACTTCCAGCACGCCTGGTCCTACACCAACGGCATCGAC |
| | | ATGAACGCGGTGCTGAAGGGCTTCCCTGAGTGCCTGCAGGCTGACATCT |
| | | GCCTGCACCTGAACCGCTCACTGCTGCAGCACTGCAAACCCTTCCGAGG |
| | | GGCCACCAAGGGCTGCCTTCGGGCCCTGGCCATGAAGTTCAAGACCAC |
| | | ACATGCACCGCCAGGGGACACACTGGTGCATGCTGGGGACCTGCTCAC |
| | | CGCCCTGTACTTCATCTCCCGGGGCTCCATCGAGATCCTGCGGGGCGAC |
| | | GTCGTCGTGGCCATCCTGGGGAAGAATGACATCTTTGGGGAGCCTCTGA |
| | | ACCTGTATGCAAGGCCTGGCAAGTCGAACGGGGATGTGCGGGCCCTCA |
| | | CCTACTGTGACCTACACAAGATCCATCGGGACGACCTGCTGGAGGTGCT |
| | | GGACATGTACCCTGAGTTCTCCGACCACTTCTGGTCCAGCCTGGAGATC |
| | | ACCTTCAACCTGCGAGATACCAACATGATCCCGGGCTCCCCCGGCAGTA |
| | | CGGAGTTAGAGGGTGGCTTCAGTCGGCAACGCAAGCGCAAGTTGTCCT |
| | | TCCGCAGGCGCACGGACAAGGACACGGAGCAGCCAGGGGAGGTGTCG |
| | | GCCTTGGGGCCGGGCCGGGCGGGGGCAGGGCCGAGTAGCCGGGGCCG |
| | | GCCGGGGGGGCCGTGGGGGGAGAGCCCGTCCAGTGGCCCCTCCAGCCC |
| | | TGAGAGCAGTGAGGATGAGGGCCCAGGCCGCAGCTCCAGCCCCCTCCG |
| | | CCTGGTGCCCTTCTCCAGCCCCAGGCCCCCCGGAGAGCCGCCGGGTGGG |
| | | GAGCCCCTGATGGAGGACTGCGAGAAGAGCAGCGACACTTGCAACCCC |
| | | CTGTCAGGCGCCTTCTCAGGAGTGTCCAACATTTTCAGCTTCTGGGGGG |
| | | ACAGTCGGGGCCGCCAGTACCAGGAGCTCCCTCGATGCCCCGCCCCCA |
| | | CCCCCAGCCTCCTCAACATCCCCCTCTCCAGCCCGGGTCGGCGGCCCCG |
| | | GGGCGACGTGGAGAGCAGGCTGGATGCCCTCCAGCGCCAGCTCAACAG |
| | | GCTGGAGACCCGGCTGAGTGCAGACATGGCCACTGTCCTGCAGCTGCT |
| | | ACAGAGGCAGATGACGCTGGTCCCGCCCGCCTACAGTGCTGTGACCAC |
| | | CCCGGGGCCTGGCCCCACTTCCACATCCCCGCTGTTGCCCGTCAGCCCC |
| | | CTCCCCACCCTCACCTTGGACTCGCTTTCTCAGGTTTCCCAGTTCATGGC |
| | | GTGTGAGGAGCTGCCCCCGGGGGCCCCAGAGCTTCCCCAAGAAGGCCC |
| | | CACACGACGCCTCTCCCTACCGGGCCAGCTGGGGGCCCTCACCTCCCAG |
| | | CCCCTGCACAGACACGGCTCGGACCCGGGCAGTTAG |
| 14 | CMV-e/Anf1-p | CGTTACATAACTTACGGTAAATGGCCCGCCTGGCTGACCGCCCAACGAC |
| | (mouse) + | CCCCGCCCATTGACGTCAATAATGACGTATGTTCCCATAGTAACGCCAA |
| | truncated G628S | TAGGGACTTTCCATTGACGTCAATGGGTGGAGTATTTACGGTAAACTGC |
| | KCNH2 | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATGCCCCCCCTCAATCCCTTACCTCCTCCTTCTATTTCCCTGG |
| | | GAAGGGTGGGACCACCACATATTTCATGCTCAGAGTCTGGGCTTTGGAA |
| | | AGAAGAAAAGTGGTTTCATCCTCCAAACCCATCCTGTTGGCACCTTGGA |
| | | CACGAGTCTTGGGAGGCAGTAAAAATAAACTTTAGAGGAAATATGCTC |
| | | TTCTAACATCCCTTGGTGTTTACACGTGTAGCTGAATTCTTTAGAGCCTG |
| | | TATCATGTTGGCTTCCTGGCTGACTTCATACTCTAAAAAAAAAATAATA |
| | | GCTCTTTCACCTGACTGCTAACAGGGACATCTAGGGTGGGGGTGGGCTG |
| | | TCTGGGGCCAGAGGTCCACCCACGAGGCCGATGAATCAGGTGTGAAGC |
| | | TAGCTCCAGCATGTGTACTCCCTGGCCAGCCTAGCTGGCCTCCCAGCTG |
| | | CCTGTCATTGCCTCCTCTCCCGCCCTTATTTGGAGCCCCTGACAGCTGAG |
| | | CAGCAAGCAGGGGGAGCTGGGTGCAGGCCAGCCGTCACCCTCTGCTTC |
| | | CCTGCATGGGTCCTGTTGCCAGGGAGAAAGAATCCTGAGGCGAGCGCC |
| | | CAGGAAGATAACCAAGGACTCTTTTCTGCTCCTCTCACACCTTTGAAGT |
| | | GGGGGCCTCTTGAGGCAAATCAGCAAGAATGTGACTCTTGCAGCTGAG |
| | | GGTCTGGGGGAGGGGGGTGAGTGGAGCTGCTCAAGGCAAAGGGGCCGT |
| | | GACAAGCTTTGCCGAACTGATAACTTTAAAAGGGCATCTTCTGCTGGCT |
| | | CCTCACTCCATCGCTTTATCGCTGCAAGTGACAGAATGGGGAGGGTTCT |
| | | AGCCCCCCTGCCTTCTCAAAGAGCTGGGGGGCTATAAAAACGGGAGAT |
| | | GCTGGCAGCTAGGAGACAGTGACGGACAAAAGCTGAGAGAGAGAGAG |
| | | AAAGAAACCAGAGTGGGCAGAGACAGCAAACATCAGATCGTGCCCCG |
| | | ACCCACGCCAGCATGCCGGTGCGGAGGGGCCACGTCGCGCCGCAGAAC |
| | | ACCTTCCTGGACACCATCATCCGCAAGTTTGAGGGCCAGAGCCGTAAGT |
| | | TCATCATCGCCAACGCTCGGGTGGAGAACTGCGCCGTCATCTACTGCAA |
| | | CGACGGCTTCTGCGAGCTGTGCGGCTACTCGCGGGCCGAGGTGATGCA |
| | | GCGACCCTGCACCTGCGACTTCCTGCACGGGCCGCGCACGCAGCGCCG |
| | | CGCTGCCGCGCAGATCGCGCAGGCACTGCTGGGCGCCGAGGAGCGCAA |
| | | AGTGGAAATCGCCTTCTACCGGAAAGATGGGAGCTGCTTCCTATGTCTG |
| | | GTGGATGTGGTGCCCGTGAAGAACGAGGATGGGGCTGTCATCATGTTC |
| | | ATCCTCAATTTCGAGGTGGTGATGGAGAAGGACATGGTGGGGTCCCCG |
| | | GCTCATGACACCAACCACCGGGGCCCCCCCACCAGCTGGCTGGCCCCA |
| | | GGCCGCGCCAAGACCTTCCGCCTGAAGCTGCCCGCGCTGCTGGCGCTGA |
| | | CGGCCCGGGAGTCGTCGGTGCGGTCGGGCGGCGCGGGCGGCGCGGGCG |
| | | CCCCGGGGGCCGTGGTGGTGGACGTGGACCTGACGCCCGCGGCACCCA |
| | | GCAGCGAGTCGCTGGCCCTGGACGAAGTGACAGCCATGGACAACCACG |
| | | TGGCAGGGCTCGGGCCCGCGGAGGAGCGGCGTGCGCTGGTGGGTCCCG |
| | | GCTCTCCGCCCCGCAGCGCGCCCGGCCAGCTCCCATCGCCCCGGGCGCA |
| | | CAGCCTCAACCCCGACGCCTCGGGCTCCAGCTGCAGCCTGGCCCGGAC |
| | | GCGCTCCCGAGAAAGCTGCGCCAGCGTGCGCCGCGCCTCGTCGGCCGA |
| | | CGACATCGAGGCCATGCGCGCCGGGGTGCTGCCCCCGCCACCGCGCCA |
| | | CGCCAGCACCGGGGCCATGCACCCACTGCGCAGCGGCTTGCTCAACTCC |
| | | ACCTCGGACTCCGACCTCGTGCGCTACCGCACCATTAGCAAGATTCCCC |
| | | AAATCACCCTCAACTTTGTGGACCTCAAGGGCGACCCCTTCTTGGCTTC |
| | | GCCCACCAGTGACCGTGAGATCATAGCACCTAAGATAAAGGAGCGAAC |
| | | CCACAATGTCACTGAGAAGGTCACCCAGGTCCTGTCCCTGGGCGCCGAC |
| | | GTGCTGCCTGAGTACAAGCTGCAGGCACCGCGCATCCACCGCTGGACC |
| | | ATCCTGCATTACAGCCCCTTCAAGGCCGTGTGGGACTGGCTCATCCTGC |
| | | TGCTGGTCATCTACACGGCTGTCTTCACACCCTACTCGGCTGCCTTCCTG |
| | | CTGAAGGAGACGGAAGAAGGCCCGCCTGCTACCGAGTGTGGCTACGCC |
| | | TGCCAGCCGCTGGCTGTGGTGGACCTCATCGTGGACATCATGTTCATTG |
| | | TGGACATCCTCATCAACTTCCGCACCACCTACGTCAATGCCAACGAGGA |
| | | GGTGGTCAGCCACCCCGGCCGCATCGCCGTCCACTACTTCAAGGGCTGG |
| | | TTCCTCATCGACATGGTGGCCGCCATCCCCTTCGACCTGCTCATCTTCGG |
| | | CTCTGGCTCTGAGGAGCTGATCGGGCTGCTGAAGACTGCGCGGCTGCTG |
| | | CGGCTGGTGCGCGTGGCGCGGAAGCTGGATCGCTACTCAGAGTACGGC |
| | | GCGGCCGTGCTGTTCTTGCTCATGTGCACCTTTGCGCTCATCGCGCACTG |
| | | GCTAGCCTGCATCTGGTACGCCATCGGCAACATGGAGCAGCCACACAT |
| | | GGACTCACGCATCGGCTGGCTGCACAACCTGGGCGACCAGATAGGCAA |
| | | ACCCTACAACAGCAGCGGCCTGGGCGGCCCCTCCATCAAGGACAAGTA |
| | | TGTGACGGCGCTCTACTTCACCTTCAGCAGCCTCACCAGTGTGGGCTTC |
| | | TCCAACGTCTCTCCCAACACCAACTCAGAGAAGATCTTCTCCATCTGCG |
| | | TCATGCTCATTGGCTCCCTCATGTATGCTAGCATCTTCGGCAACGTGTCG |
| | | GCCATCATCCAGCGGCTGTACTCGGGCACAGCCCGCTACCACACACAG |
| | | ATGCTGCGGGTGCGGGAGTTCATCCGCTTCCACCAGATCCCCAATCCCC |
| | | TGCGCCAGCGCCTCGAGGAGTACTTCCAGCACGCCTGGTCCTACACCAA |
| | | CGGCATCGACATGAACGCGGTGCTGAAGGGCTTCCCTGAGTGCCTGCA |
| | | GGCTGACATCTGCCTGCACCTGAACCGCTCACTGCTGCAGCACTGCAAA |
| | | CCCTTCCGAGGGGCCACCAAGGGCTGCCTTCGGGCCCTGGCCATGAAGT |
| | | TCAAGACCACACATGCACCGCCAGGGGACACACTGGTGCATGCTGGGG |
| | | ACCTGCTCACCGCCCTGTACTTCATCTCCCGGGGCTCCATCGAGATCCT |
| | | GCGGGGCGACGTCGTCGTGGCCATCCTGGGGAAGAATGACATCTTTGG |
| | | GGAGCCTCTGAACCTGTATGCAAGGCCTGGCAAGTCGAACGGGGATGT |
| | | GCGGGCCCTCACCTACTGTGACCTACACAAGATCCATCGGGACGACCTG |
| | | CTGGAGGTGCTGGACATGTACCCTGAGTTCTCCGACCACTTCTGGTCCA |
| | | GCCTGGAGATCACCTTCAACCTGCGAGATACCAACATGATCCCGGGCTC |
| | | CCCCGGCAGTACGGAGTTAGAGGGTGGCTTCAGTCGGCAACGCAAGCG |
| | | CAAGTTGTCCTTCCGCAGGCGCACGGACAAGGACACGGAGCAGCCAGG |
| | | GGAGGTGTCGGCCTTGGGGCCGGGCCGGGCGGGGGCAGGGCCGAGTAG |
| | | CCGGGGCTAG |
| 15 | CMV-e/Anf1-p | CGTTACATAACTTACGGTAAATGGCCCGCCTGGCTGACCGCCCAACGAC |
| | (human) + | CCCCGCCCATTGACGTCAATAATGACGTATGTTCCCATAGTAACGCCAA |
| | truncated G628S | TAGGGACTTTCCATTGACGTCAATGGGTGGAGTATTTACGGTAAACTGC |
| | KCNH2 | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATGCCTCCTTCACCCCCACCTCCCTTCCTCCATCGGTCAAGTT |
| | | GCCACGGGCACAAGCCCAGGAAGGGCAGGGGGATGTTAACAAACTTCA |
| | | GACACGAAGAGTCTGAGCATCTATAAACAGCAACGGAAGAAATGAAAT |
| | | TGGCTGCGTCCTCTAAGCCTGTCCCCGCAGCATGCTGGAGGAGGGTCGC |
| | | GGGGGACATGGAAGAGGAGGAGCTTTGGAGAGAGGATGCTTGTGCTCC |
| | | CCCGCCTTTTCTTGCTATTTCTATTTGGGGGTTGGATTCCTGGGAGCTTC |
| | | ATCACATTTGGTTCTCAGCTGACTTTATATACTAAAAAATAACTTCCTTT |
| | | CGCCTGACCATGGAGAGGGACTGCCAGGGGTGAAGGCAGCCCTGTCTG |
| | | AGGCCAGAGGTCTGCCCACGTGGCGGATGAGGCAGGTGTGAGGCCAGC |
| | | TTGAGCATCTGGATCCATTTGTCTCGGGCTGCTGGCTGCCTGCCATTTCC |
| | | TCCTCTCCACCCTTATTTGGAGGCCCTGACAGCTGAGCCACAAACAAAC |
| | | CAGGGGAGCTGGGCACCAGCCAAGCGTCACCCTCTGTTTCCCCGCACG |
| | | GGTACCAGCGTCGAGGAGAAAGAATCCTGAGGCACGGCGGTGAGATAA |
| | | CCAAGGACTCTTTTTTACTCTTCTCACACCTTTGAAGTGGGAGCCTCTTG |
| | | AGTCAAATCAGTAAGAATGCGGCTCTTGCAGCTGAGGGTCTGGGGGGC |
| | | TGTTGGGGCTGCCCAAGGCAGAGAGGGGCTGTGACAAGCCCTGCGGGA |
| | | TGATAACTTTAAAAGGGCATCTCCTGCTGGCTTCTCACTTGGCAGCTTT |
| | | ATCACTGCAAGTGACAGAATGGGGAGGGTTCTGTCTCTCCTGCGTGCTT |
| | | GGAGAGCTGGGGGGCTATAAAAAGAGGCGGCACTGGGCAGCTGGGAG |
| | | ACAGGGACAGACGTAGGCCAAGAGAGGGGAACCAGAGAGGAACCAGA |
| | | GGGGAGAGACAGAGCAGCAAGCAGTGGATTGCTCCTTGACGACGCCAG |
| | | ATGCCGGTGCGGAGGGGCCACGTCGCGCCGCAGAACACCTTCCTGGAC |
| | | ACCATCATCCGCAAGTTTGAGGGCCAGAGCCGTAAGTTCATCATCGCCA |
| | | ACGCTCGGGTGGAGAACTGCGCCGTCATCTACTGCAACGACGGCTTCTG |
| | | CGAGCTGTGCGGCTACTCGCGGGCCGAGGTGATGCAGCGACCCTGCAC |
| | | CTGCGACTTCCTGCACGGGCCGCGCACGCAGCGCCGCGCTGCCGCGCA |
| | | GATCGCGCAGGCACTGCTGGGCGCCGAGGAGCGCAAAGTGGAAATCGC |
| | | CTTCTACCGGAAAGATGGGAGCTGCTTCCTATGTCTGGTGGATGTGGTG |
| | | CCCGTGAAGAACGAGGATGGGGCTGTCATCATGTTCATCCTCAATTTCG |
| | | AGGTGGTGATGGAGAAGGACATGGTGGGGTCCCCGGCTCATGACACCA |
| | | ACCACCGGGGCCCCCCCACCAGCTGGCTGGCCCCAGGCCGCGCCAAGA |
| | | CCTTCCGCCTGAAGCTGCCCGCGCTGCTGGCGCTGACGGCCCGGGAGTC |
| | | GTCGGTGCGGTCGGGCGGCGCGGGCGGCGCGGGCGCCCCGGGGGCCGT |
| | | GGTGGTGGACGTGGACCTGACGCCCGCGGCACCCAGCAGCGAGTCGCT |
| | | GGCCCTGGACGAAGTGACAGCCATGGACAACCACGTGGCAGGGCTCGG |
| | | GCCCGCGGAGGAGCGGCGTGCGCTGGTGGGTCCCGGCTCTCCGCCCCG |
| | | CAGCGCGCCCGGCCAGCTCCCATCGCCCCGGGCGCACAGCCTCAACCC |
| | | CGACGCCTCGGGCTCCAGCTGCAGCCTGGCCCGGACGCGCTCCCGAGA |
| | | AAGCTGCGCCAGCGTGCGCCGCGCCTCGTCGGCCGACGACATCGAGGC |
| | | CATGCGCGCCGGGGTGCTGCCCCCGCCACCGCGCCACGCCAGCACCGG |
| | | GGCCATGCACCCACTGCGCAGCGGCTTGCTCAACTCCACCTCGGACTCC |
| | | GACCTCGTGCGCTACCGCACCATTAGCAAGATTCCCCAAATCACCCTCA |
| | | ACTTTGTGGACCTCAAGGGCGACCCCTTCTTGGCTTCGCCCACCAGTGA |
| | | CCGTGAGATCATAGCACCTAAGATAAAGGAGCGAACCCACAATGTCAC |
| | | TGAGAAGGTCACCCAGGTCCTGTCCCTGGGCGCCGACGTGCTGCCTGAG |
| | | TACAAGCTGCAGGCACCGCGCATCCACCGCTGGACCATCCTGCATTACA |
| | | GCCCCTTCAAGGCCGTGTGGGACTGGCTCATCCTGCTGCTGGTCATCTA |
| | | CACGGCTGTCTTCACACCCTACTCGGCTGCCTTCCTGCTGAAGGAGACG |
| | | GAAGAAGGCCCGCCTGCTACCGAGTGTGGCTACGCCTGCCAGCCGCTG |
| | | GCTGTGGTGGACCTCATCGTGGACATCATGTTCATTGTGGACATCCTCA |
| | | TCAACTTCCGCACCACCTACGTCAATGCCAACGAGGAGGTGGTCAGCC |
| | | ACCCCGGCCGCATCGCCGTCCACTACTTCAAGGGCTGGTTCCTCATCGA |
| | | CATGGTGGCCGCCATCCCCTTCGACCTGCTCATCTTCGGCTCTGGCTCTG |
| | | AGGAGCTGATCGGGCTGCTGAAGACTGCGCGGCTGCTGCGGCTGGTGC |
| | | GCGTGGCGCGGAAGCTGGATCGCTACTCAGAGTACGGCGCGGCCGTGC |
| | | TGTTCTTGCTCATGTGCACCTTTGCGCTCATCGCGCACTGGCTAGCCTGC |
| | | ATCTGGTACGCCATCGGCAACATGGAGCAGCCACACATGGACTCACGC |
| | | ATCGGCTGGCTGCACAACCTGGGCGACCAGATAGGCAAACCCTACAAC |
| | | AGCAGCGGCCTGGGCGGCCCCTCCATCAAGGACAAGTATGTGACGGCG |
| | | CTCTACTTCACCTTCAGCAGCCTCACCAGTGTGGGCTTCTCCAACGTCTC |
| | | TCCCAACACCAACTCAGAGAAGATCTTCTCCATCTGCGTCATGCTCATT |
| | | GGCTCCCTCATGTATGCTAGCATCTTCGGCAACGTGTCGGCCATCATCC |
| | | AGCGGCTGTACTCGGGCACAGCCCGCTACCACACACAGATGCTGCGGG |
| | | TGCGGGAGTTCATCCGCTTCCACCAGATCCCCAATCCCCTGCGCCAGCG |
| | | CCTCGAGGAGTACTTCCAGCACGCCTGGTCCTACACCAACGGCATCGAC |
| | | ATGAACGCGGTGCTGAAGGGCTTCCCTGAGTGCCTGCAGGCTGACATCT |
| | | GCCTGCACCTGAACCGCTCACTGCTGCAGCACTGCAAACCCTTCCGAGG |
| | | GGCCACCAAGGGCTGCCTTCGGGCCCTGGCCATGAAGTTCAAGACCAC |
| | | ACATGCACCGCCAGGGGACACACTGGTGCATGCTGGGGACCTGCTCAC |
| | | CGCCCTGTACTTCATCTCCCGGGGCTCCATCGAGATCCTGCGGGGCGAC |
| | | GTCGTCGTGGCCATCCTGGGGAAGAATGACATCTTTGGGGAGCCTCTGA |
| | | ACCTGTATGCAAGGCCTGGCAAGTCGAACGGGGATGTGCGGGCCCTCA |
| | | CCTACTGTGACCTACACAAGATCCATCGGGACGACCTGCTGGAGGTGCT |
| | | GGACATGTACCCTGAGTTCTCCGACCACTTCTGGTCCAGCCTGGAGATC |
| | | ACCTTCAACCTGCGAGATACCAACATGATCCCGGGCTCCCCCGGCAGTA |
| | | CGGAGTTAGAGGGTGGCTTCAGTCGGCAACGCAAGCGCAAGTTGTCCT |
| | | TCCGCAGGCGCACGGACAAGGACACGGAGCAGCCAGGGGAGGTGTCG |
| | | GCCTTGGGGCCGGGCCGGGCGGGGGCAGGGCCGAGTAGCCGGGGCTAG |
| 16 | Anf1-e | CTCAGCCGTTCTTCAAGTCTTTGCCCAACTCAAAGACCATCTCCAGGAG |
| | (mouse)/CMV-p | GTAGGTCATGTGACACACACAGCAAGGAGCCCGAGTTCCCCCAGTAGG |
| | hybrid regulatory | GCTGCTCGCCTAGTCCTCTCTGCTAATAATAATCCCCAGGCACACAATT |
| | element | AAGCAGTGAGCGCATCCCAGGGACTCATCCGGAGGCGACAGTGTGAAC |
| | | TTGCACAGATTTCAGACTTCAAGGAGAGATTGGCCAACCCAAATTGGTT |
| | | GCCTACAACCCCCAGCTTTCTCAAAGGGCTCTCTTTTTCTATCTTAGAGA |
| | | AAACATCACCGCAGACACCTCTTGTGACTTCCAGCCACATGACAACTCC |
| | | ACCAGGCAGCCACAGGGTCAATGGGCGTGGAAGTCTTCCAATAGCCCA |
| | | TTAAGACGGTCAGAAAGCAGAGAGCAGCAGAAAATGCACACACTTATC |
| | | ACACACACAAAGCATACATATGTCGAAAATGGCTACACTGGGGTGTGG |
| | | AGACCACTTGTTAAAGGTGGACTGCCTGCTCATGCTCCTGAGGCTGCCA |
| | | GAAGAATTCATGGCTCACTTCCTTGGTCTCAAAGGTCATGAAGGACATT |
| | | ATGGAGAATGGTGTATGTGTGTGATGCGGTTTTGGCAGTACATCAATGG |
| | | GCGTGGATAGCGGTTTGACTCACGGGGATTTCCAAGTCTCCACCCCATT |
| | | GACGTCAATGGGAGTTTGTTTTGGCACCAAAATCAACGGGACTTTCCAA |
| | | AATGTCGTAACAACTCCGCCCCATTGACGCAAATGGGCGGTAGGCGTG |
| | | TACGGTGGGAGGTCTATATAAGCAGAGCTCATCGCTATTACC |
| 17 | Anf1-e/Anf1-p | CTCAGCCGTTCTTCAAGTCTTTGCCCAACTCAAAGACCATCTCCAGGAG |
| | (mouse)(1.58-kb | GTAGGTCATGTGACACACACAGCAAGGAGCCCGAGTTCCCCCAGTAGG |
| | regulatory | GCTGCTCGCCTAGTCCTCTCTGCTAATAATAATCCCCAGGCACACAATT |
| | element) | AAGCAGTGAGCGCATCCCAGGGACTCATCCGGAGGCGACAGTGTGAAC |
| | | TTGCACAGATTTCAGACTTCAAGGAGAGATTGGCCAACCCAAATTGGTT |
| | | GCCTACAACCCCCAGCTTTCTCAAAGGGCTCTCTTTTTCTATCTTAGAGA |
| | | AAACATCACCGCAGACACCTCTTGTGACTTCCAGCCACATGACAACTCC |
| | | ACCAGGCAGCCACAGGGTCAATGGGCGTGGAAGTCTTCCAATAGCCCA |
| | | TTAAGACGGTCAGAAAGCAGAGAGCAGCAGAAAATGCACACACTTATC |
| | | ACACACACAAAGCATACATATGTCGAAAATGGCTACACTGGGGTGTGG |
| | | AGACCACTTGTTAAAGGTGGACTGCCTGCTCATGCTCCTGAGGCTGCCA |
| | | GAAGAATTCATGGCTCACTTCCTTGGTCTCAAAGGTCATGAAGGACATT |
| | | ATGGAGAATGGTGTATGTGTCCCCCCCTCAATCCCTTACCTCCTCCTTCT |
| | | ATTTCCCTGGGAAGGGTGGGACCACCACATATTTCATGCTCAGAGTCTG |
| | | GGCTTTGGAAAGAAGAAAAGTGGTTTCATCCTCCAAACCCATCCTGTTG |
| | | GCACCTTGGACACGAGTCTTGGGAGGCAGTAAAAATAAACTTTAGAGG |
| | | AAATATGCTCTTCTAACATCCCTTGGTGTTTACACGTGTAGCTGAATTCT |
| | | TTAGAGCCTGTATCATGTTGGCTTCCTGGCTGACTTCATACTCTAAAAA |
| | | AAAAATAATAGCTCTTTCACCTGACTGCTAACAGGGACATCTAGGGTGG |
| | | GGGTGGGCTGTCTGGGGCCAGAGGTCCACCCACGAGGCCGATGAATCA |
| | | GGTGTGAAGCTAGCTCCAGCATGTGTACTCCCTGGCCAGCCTAGCTGGC |
| | | CTCCCAGCTGCCTGTCATTGCCTCCTCTCCCGCCCTTATTTGGAGCCCCT |
| | | GACAGCTGAGCAGCAAGCAGGGGGAGCTGGGTGCAGGCCAGCCGTCAC |
| | | CCTCTGCTTCCCTGCATGGGTCCTGTTGCCAGGGAGAAAGAATCCTGAG |
| | | GCGAGCGCCCAGGAAGATAACCAAGGACTCTTTTCTGCTCCTCTCACAC |
| | | CTTTGAAGTGGGGGCCTCTTGAGGCAAATCAGCAAGAATGTGACTCTTG |
| | | CAGCTGAGGGTCTGGGGGAGGGGGGTGAGTGGAGCTGCTCAAGGCAAA |
| | | GGGGCCGTGACAAGCTTTGCCGAACTGATAACTTTAAAAGGGCATCTTC |
| | | TGCTGGCTCCTCACTCCATCGCTTTATCGCTGCAAGTGACAGAATGGGG |
| | | AGGGTTCTAGCCCCCCTGCCTTCTCAAAGAGCTGGGGGGCTATAAAAAC |
| | | GGGAGATGCTGGCAGCTAGGAGACAGTGACGGACAAAAGCTGAGAGA |
| | | GAGAGAGAAAGAAACCAGAGTGGGCAGAGACAGCAAACATCAGATCG |
| | | TGCCCCGACCCACGCCAGC |
| 18 | CMV-e/CMV-p | CGTTACATAACTTACGGTAAATGGCCCGCCTGGCTGACCGCCCAACGAC |
| | regulatory | CCCCGCCCATTGACGTCAATAATGACGTATGTTCCCATAGTAACGCCAA |
| | element | TAGGGACTTTCCATTGACGTCAATGGGTGGAGTATTTACGGTAAACTGC |
| | | CCACTTGGCAGTACATCAAGTGTATCATATGCCAAGTACGCCCCCTATT |
| | | GACGTCAATGACGGTAAATGGCCCGCCTGGCATTATGCCCAGTACATG |
| | | ACCTTATGGGACTTTCCTACTTGGCAGTACATCTACGTATTAGTCATCGC |
| | | TATTACCATGGTGATGCGGTTTTGGCAGTACATCAATGGGCGTGGATAG |
| | | CGGTTTGACTCACGGGGATTTCCAAGTCTCCACCCCATTGACGTCAATG |
| | | GGAGTTTGTTTTGGCACCAAAATCAACGGGACTTTCCAAAATGTCGTAA |
| | | CAACTCCGCCCCATTGACGCAAATGGGCGGTAGGCGTGTACGGTGGGA |
| | | GGTCTATATAAGCAGAGCTCATCGCTATTACC |

TABLE 2
Amino Acid Sequences

| SEQ | | |
|---|---|---|
| ID | Amino acid | |
| NO | sequence | Sequence (N-term to C-term) |
| 19 | Wild-type | MPVRRGHVAPQNTFLDTIIRKFEGQSRKFIIANARVENCAVIYCNDGFCELC |
| | KCNH2 (full- | GYSRAEVMQRPCTCDFLHGPRTQRRAAAQIAQALLGAEERKVEIAFYRKD |
| | length) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 20 | G628S KCNH2 | MPVRRGHVAPQNTFLDTIIRKFEGQSRKFIIANARVENCAVIYCNDGFCELC |
| | (full-length) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFSNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 21 | Wild-type | MPVRRGHVAPQNTFLDTIIRKFEGQSRKFIIANARVENCAVIYCNDGFCELC |
| | KCNH2 | GYSRAEVMQRPCTCDFLHGPRTQRRAAAQIAQALLGAEERKVEIAFYRKD |
| | (truncated) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 22 | G628S KCNH2 | MPVRRGHVAPQNTFLDTIIRKFEGQSRKFIIANARVENCAVIYCNDGFCELC |
| | (truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFSNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 23 | N470D KCNH2 | MPVRRGHVAPQNTFLDTIIRKFEGQSRKFIIANARVENCAVIYCNDGFCELC |
| | (full-length) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILIDFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 24 | N470D KCNH2 | MPVRRGHVAPQNTFLDTIIRKFEGQSRKFIIANARVENCAVIYCNDGFCELC |
| | (truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILIDFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 25 | N470E | MPVRRGHVAPQNTFLDTIIRKFEGQSRKFIIANARVENCAVIYCNDGFCELC |
| | KCNH2 (full- | GYSRAEVMQRPCTCDFLHGPRTQRRAAAQIAQALLGAEERKVEIAFYRKD |
| | length) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILIEFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 26 | N470E | MPVRRGHVAPQNTFLDTIIRKFEGQSRKFIIANARVENCAVIYCNDGFCELC |
| | KCNH2 | GYSRAEVMQRPCTCDFLHGPRTQRRAAAQIAQALLGAEERKVEIAFYRKD |
| | (truncated) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILIEFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 27 | T473P KCNH2 | MPVRRGHVAPQNTFLDTIIRKFEGQSRKFIIANARVENCAVIYCNDGFCELC |
| | (full-length) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRPTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 28 | T473P KCNH2 | MPVRRGHVAPQNTFLDTIIRKFEGQSRKFIIANARVENCAVIYCNDGFCELC |
| | (truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRPTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 29 | A561V | MPVRRGHVAPQNTFLDTIIRKFEGQSRKFIIANARVENCAVIYCNDGFCELC |
| | KCNH2 (full- | GYSRAEVMQRPCTCDFLHGPRTQRRAAAQIAQALLGAEERKVEIAFYRKD |
| | length) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIVHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 30 | A561V | MPVRRGHVAPQNTFLDTIIRKFEGQSRKFIIANARVENCAVIYCNDGFCELC |
| | KCNH2 | GYSRAEVMQRPCTCDFLHGPRTQRRAAAQIAQALLGAEERKVEIAFYRKD |
| | (truncated) | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIVHWLAC |
| | | IWYAIGNMEQPHMDSRIGWLHNLGDQIGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 31 | I593R KCNH2 | MPVRRGHVAPQNTFLDTIIRKFEGQSRKFIIANARVENCAVIYCNDGFCELC |
| | (full-length) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQRGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 32 | I593R KCNH2 | MPVRRGHVAPQNTFLDTIIRKFEGQSRKFIIANARVENCAVIYCNDGFCELC |
| | (truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQRGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 33 | I593K KCNH2 | MPVRRGHVAPQNTFLDTIIRKFEGQSRKFIIANARVENCAVIYCNDGFCELC |
| | (full-length) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQKGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRGRPGG |
| | | PWGESPSSGPSSPESSEDEGPGRSSSPLRLVPFSSPRPPGEPPGGEPLMEDCE |
| | | KSSDTCNPLSGAFSGVSNIFSFWGDSRGRQYQELPRCPAPTPSLLNIPLSSPG |
| | | RRPRGDVESRLDALQRQLNRLETRLSADMATVLQLLQRQMTLVPPAYSAV |
| | | TTPGPGPTSTSPLLPVSPLPTLTLDSLSQVSQFMACEELPPGAPELPQEGPTR |
| | | RLSLPGQLGALTSQPLHRHGSDPGS |
| 34 | I593K KCNH2 | MPVRRGHVAPQNTFLDTIIRKFEGQSRKFIIANARVENCAVIYCNDGFCELC |
| | (truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLIDMVAAIPFDLLIFGS |
| | | GSEELIGLLKTARLLRLVRVARKLDRYSEYGAAVLFLLMCTFALIAHWLAC |
| | | IWYAIGNMEQPHMDSRIGWLHNLGDQKGKPYNSSGLGGPSIKDKYVTALY |
| | | FTFSSLTSVGFGNVSPNTNSEKIFSICVMLIGSLMYASIFGNVSAIIQRLYSGT |
| | | ARYHTQMLRVREFIRFHQIPNPLRQRLEEYFQHAWSYTNGIDMNAVLKGF |
| | | PECLQADICLHLNRSLLQHCKPFRGATKGCLRALAMKFKTTHAPPGDTLV |
| | | HAGDLLTALYFISRGSIEILRGDVVVAILGKNDIFGEPLNLYARPGKSNGDV |
| | | RALTYCDLHKIHRDDLLEVLDMYPEFSDHFWSSLEITFNLRDTNMIPGSPGS |
| | | TELEGGFSRQRKRKLSFRRRTDKDTEQPGEVSALGPGRAGAGPSSRG |
| 35 | KCNH2 ΔA500- | MPVRRGHVAPQNTFLDTIIRKFEGQSRKFIIANARVENCAVIYCNDGFCELC |
| | 508(truncated) | GYSRAEVMQRPCTCDFLHGPRTQRRAAAQIAQALLGAEERKVEIAFYRKD |
| | | GSCFLCLVDVVPVKNEDGAVIMFILNFEVVMEKDMVGSPAHDTNHRGPPT |
| | | SWLAPGRAKTFRLKLPALLALTARESSVRSGGAGGAGAPGAVVVDVDLTP |
| | | AAPSSESLALDEVTAMDNHVAGLGPAEERRALVGPGSPPRSAPGQLPSPRA |
| | | HSLNPDASGSSCSLARTRSRESCASVRRASSADDIEAMRAGVLPPPPRHAST |
| | | GAMHPLRSGLLNSTSDSDLVRYRTISKIPQITLNFVDLKGDPFLASPTSDREII |
| | | APKIKERTHNVTEKVTQVLSLGADVLPEYKLQAPRIHRWTILHYSPFKAVW |
| | | DWLILLLVIYTAVFTPYSAAFLLKETEEGPPATECGYACQPLAVVDLIVDIM |
| | | FIVDILINFRTTYVNANEEVVSHPGRIAVHYFKGWFLDLLIFGSGSEELIGLL |
| | | KTARLLRLVRVARKLDRYSEYGAAVLFLLMCTFALIAHWLACIWYAIGNM |
| | | EQPHMDSRIGWLHNLGDQIGKPYNSSGLGGPSIKDKYVTALYFTFSSLTSV |
| | | GFGNVSPNTNSEKIFSICVMLIGSLMYASIFGNVSAIIQRLYSGTARYHTQM |
| | | LRVREFIRFHQIPNPLRQRLEEYFQHAWSYTNGIDMNAVLKGFPECLQADI |
| | | CLHLNRSLLQHCKPFRGATKGCLRALAMKFKTTHAPPGDTLVHAGDLLTA |
| | | LYFISRGSIEILRGDVVVAILGKNDIFGEPLNLYARPGKSNGDVRALTYCDL |
| | | HKIHRDDLLEVLDMYPEFSDHFWSSLEITFNLRDTNMIPGSPGSTELEGGFS |
| | | RQRKRKLSFRRRTDKDTEQPGEVSALGPGRAGAGPSSRG |

### Additional Embodiments

Additional embodiments of the present disclosure are encompassed by the following numbered paragraphs:
1. A truncated potassium voltage-gated channel subfamily H member 2 (KCNH2) protein comprising one or more dominant-negative mutations relative to a wild-type KCNH2 protein and having a length of 500 to 1,150 amino acids.
2. The truncated KCNH2 protein of paragraph 1, wherein the one or more dominant-negative mutations comprises one or more amino acid substitutions.
3. The truncated KCNH2 protein of paragraph 2, wherein the one or more amino acid substitutions is at position N470, T473, A561, I593, G626, F627, and/or G628 relative to amino acid position numbering of a wild-type KCNH2 protein (e.g., SEQ ID NO: 19).
4. The truncated KCNH2 protein of paragraph 3, wherein the amino acid substitution at position G628 is G628S, G628A, G628R, G628N, G628D, G628C, G628Q, G628E, G628H, G628I, G628L, G628K, G628M, G628F, G628P, G628S, G628T, G628W, G628Y, or G628V.
5. The truncated KCNH2 protein of paragraph 3, wherein the amino acid substitution at position N470 is N470D or N470E.
6. The truncated KCNH2 protein of paragraph 3, wherein the amino acid substitution at position T473 is T473P.
7. The truncated KCNH2 protein of paragraph 3, wherein the amino acid substitution at position A561 is A561V.
8. The truncated KCNH2 protein of paragraph 3, wherein the amino acid substitution at position I593 is I593R or I593K.
9. The truncated KCNH2 protein of paragraph 3, wherein the amino acid substitution at position G626 is G626S, G626A, G626R, G626N, G626D, G626C, G626Q, G626E, G626H, G626I, G626L, G626K, G626M, G626F, G626P, G626T, G626W, G626Y, or G626V.
10. The truncated KCNH2 protein of paragraph 3, wherein the amino acid substitution at position F627 is F627S, F627A, F627R, F627N, F627D, F627C, F627Q, F627E, F627H, F627I, F627L, F627K, F627M, F627G, F627P, F627T, F627W, F627Y, or F627V.
11. The truncated KCNH2 protein of paragraph 2, wherein the one or more dominant-negative mutations comprises a deletion.
12. The truncated KCNH2 protein of paragraph 11, wherein the deletion comprises a deletion of residues 500-508 relative to amino acid position numbering of a wild-type KCNH2 protein.
13. The truncated KCNH2 protein of any one of paragraphs 1-12, wherein the protein is truncated by at least 10, 20, 30, 50, 100, 150, 200, 250, or more amino acids compared to the amino acid sequence set forth in SEQ ID NO: 19.
14. The truncated KCNH2 protein of any one of paragraphs 1-13, wherein the protein consists of 500-1,000 amino acids, 500-900 amino acids, or 500-800 amino acids.
15. The truncated KCNH2 protein of paragraph 14 consisting of 650-950 amino acids, optionally 910-930 amino acids.
16. The truncated KCNH2 protein of any one of paragraphs 1-15 comprising at least 80%, 85%, 90%, 95%, 97%, 98%, or 99% identity to the amino acid sequence of SEQ ID NO: 21.
17. The truncated KCNH2 protein of any one of paragraphs 1-16 comprising or consisting of the amino acid sequence of SEQ ID NO: 22, 24, 26, 28, 30, 32, 34, or 35.
18. The truncated KCNH2 protein of any one of paragraphs 1-17, wherein the protein suppresses delayed rectifier potassium current (I_{Kr}) channel function.
19. An isolated nucleic acid encoding the truncated KCNH2 protein of any one of paragraphs 1-18.
20. The isolated nucleic acid of paragraph 19 comprising a nucleic acid sequence that is at least 70% identical to the nucleic acid sequence set forth in SEQ ID NOs: 1 or 2.
21. The isolated nucleic acid of paragraph 19 or 20 comprising a nucleic acid sequence that is at least 60%, 70%, 80%, 90%, 95%, 99%, or 99.9% identical to the nucleic acid sequence set forth in SEQ ID NOs: 1 or 2.
22. The isolated nucleic acid of paragraph 21 comprising a nucleic acid sequence that is 100% identical to the nucleic acid sequence set forth in SEQ ID NO: 2.
23. The isolated nucleic acid of any one of paragraphs 19-22, further comprising a Kozak sequence.
24. The isolated nucleic acid of any one of paragraphs 19-23, further comprising one or more regulatory elements.
25. The isolated nucleic acid of paragraph 24, wherein the one or more regulatory elements comprises a promoter, optionally wherein the promoter is an atrial cell-specific promoter, further optionally wherein the atrial cell-specific promoter comprises an Anf1 promoter sequence, optionally wherein the promoter sequence is set forth in SEQ ID NO: 3.
26. The isolated nucleic acid of paragraph 24 or 25, wherein the one or more regulatory elements comprises an enhancer, optionally wherein the enhancer comprises a CMV enhancer sequence, optionally wherein the enhancer sequence is set forth in SEQ ID NO: 4.
27. The isolated nucleic acid of any one of paragraphs 24-26, further comprising one or more introns, optionally wherein the one or more introns is positioned between the one or more regulatory element and a nucleic acid sequence encoding the truncated KCNH2 protein.
28. The isolated nucleic acid of any one of paragraphs 19-27, further comprising a 3′ untranslated region (3′ UTR).
29. The isolated nucleic acid of any one of paragraphs 19-28, further comprising a 5′ untranslated region (5′ UTR).
30. The isolated nucleic acid of any one of paragraphs 19-29, further comprising adeno-associated virus (AAV) inverted terminal repeats (ITRs) flanking a nucleotide sequence encoding the truncated KCNH2 protein.
31. The isolated nucleic acid of paragraph 30, wherein the AAV ITRs are of serotype AAV1, AAV2, AAV6, AAV8, or AAV9.
32. A vector comprising the isolated nucleic acid of any one of paragraphs 19-31.
33. The vector of paragraph 32, wherein the vector is a plasmid or a viral vector.
34. The vector of paragraph 33, wherein the viral vector is an adenoviral vector, adeno-associated viral vector, a lentiviral vector, a retroviral vector, or a Baculovirus vector.
35. A host cell comprising the truncated KCNH2 protein of any one of paragraphs 1-18, the isolated nucleic acid of any one of paragraphs 19-31, and/or the vector of any one of paragraphs 32-34.
36. The host cell of paragraph 35, wherein the host cell is a mammalian cell, yeast cell, bacterial cell, or insect cell.
37. A recombinant adeno-associated virus (rAAV) comprising:
(i) an isolated nucleic acid encoding the truncated KCNH2 protein of any one of paragraphs 1-18; and
(ii) an adeno-associated virus (AAV) capsid protein.
38. The rAAV of paragraph 37, wherein the capsid protein has a tropism for heart tissue, optionally wherein the heart tissue is atrial tissue.
39. The rAAV of paragraph 37 or 38, wherein the capsid protein is of a serotype selected from AAV1, AAV2, AAV3, AAV4, AAV5, AAV6, AAV7, AAV8, AAV9, and a variant of any of the foregoing.
40. The rAAV of paragraph 39, wherein the capsid protein is of serotype AAV1.
41. The rAAV of any one of paragraphs 37-40, wherein the rAAV is formulated for delivery to heart tissue of a subject.
42. A composition comprising the truncated KCNH2 protein of any one of paragraphs 1-18, the isolated nucleic acid of any one of paragraphs 19-31, the vector of any one of paragraphs 32-34, and/or the rAAV of any one of paragraphs 37-41.
43. The composition of paragraph 42, further comprising a pharmaceutically acceptable excipient.
44. A method of administering a truncated KCNH2 protein to a subject, the method comprising administering to the subject the isolated nucleic acid of any one of paragraphs 18-30, the rAAV of any one of paragraphs 36-40, and/or the composition of paragraph 42 or 43.
45. A method of decreasing activity of wild-type KCNH2 in a subject, the method comprising administering to the subject the isolated nucleic acid of any one of paragraphs 1-18, the rAAV of any one of paragraphs 37-41, and/or composition of paragraph 42 or 43.
46. The method of paragraph 44 or 45, wherein the subject has a cardiac disorder.
47. The method of any one of paragraphs 44-46, wherein the subject has atrial fibrillation.
48. A method of treating atrial fibrillation in a subject in need thereof, comprising administering to the subject the composition of paragraph 42 or 43.
49. The method of any one of paragraphs 44-48, wherein the subject is human.
50. The method of any one of paragraphs 44-49, wherein the administering is via gene painting.
51. The method of any one of paragraphs 44-50, wherein the administration is via injection, optionally intravenous injection.
52. A method of decreasing activity of wild-type KCNH2 in a cell, comprising administering to the cell the truncated KCNH2 protein of any one of paragraphs 1-18, the isolated nucleic acid of any one of paragraphs 19-31, the vector of any one of paragraphs 32-34, the rAAV of any one of paragraphs 37-41, and/or the composition of paragraph 42 or 43.
53. The method of paragraph 52, wherein the cell is cultured in vitro.
54. A nucleic acid regulatory element comprising (i) an atrial natriuretic peptide (Anf) promoter and (ii) an enhancer element, wherein the enhancer element is not a naturally-occurring Anf enhancer element.
55. The nucleic acid regulatory element of paragraph 54, wherein the naturally-occurring Anf enhancer element consists of the nucleic acid sequence set forth in SEQ ID NO: 6.
56. The nucleic acid regulatory element of paragraph 54, wherein the Anf promoter is a mouse Anf promoter or a human Anf promoter.
57. The nucleic acid regulatory element of paragraph 56, wherein the mouse Anf promoter comprises a nucleic acid sequence that is at least 70%, 80%, 90%, 95%, 97%, 98%, or 99% identical to the nucleic acid sequence set forth in SEQ ID NO: 3.
58. The nucleic acid regulatory element of paragraph 57, wherein the mouse Anf promoter comprises or consists of the nucleic acid sequence set forth in SEQ ID NO: 3.
59. The nucleic acid regulatory element of paragraph 56, wherein the human Anf promoter comprises a nucleic acid sequence that is at least 70%, 80%, 90%, 95%, 97%, 98%, or 99% identical to the nucleic acid sequence set forth in SEQ ID NO: 8.
60. The nucleic acid regulatory element of paragraph 59, wherein the human Anf promoter comprises or consists of the nucleic acid sequence as set forth in SEQ ID NO: 8.
61. The nucleic acid regulatory element of any one of paragraphs 54-60, wherein the enhancer element is a CMV enhancer.
62. The nucleic acid regulatory element of paragraph 61, wherein the CMV enhancer comprises a nucleic acid sequence that is at least 70%, 80%, 90%, 95%, 97%, 98%, or 99% identical to the nucleic acid sequence set forth in SEQ ID NO: 4.
63. The nucleic acid regulatory element of paragraph 62, wherein the CMV enhancer comprises or consists of the nucleic acid sequence as set forth in SEQ ID NO: 4.
64. The nucleic acid regulatory element of any one of paragraphs 54-63 comprising a nucleic acid that is at least 70%, 80%, 90%, 95%, 97%, 98%, or 99% identical to the nucleic acid sequence set forth in SEQ ID NOs: 5 or 9.
65. The nucleic acid regulatory element of paragraph 64 comprising or consisting of the nucleic acid sequence as set forth in SEQ ID NOs: 5 or 9.
66. An isolated nucleic acid comprising the nucleic acid regulatory element of any one of paragraphs 54-65 operably linked to a nucleic acid sequence encoding a protein.
67. The isolated nucleic acid of paragraph 66, wherein the protein is a protein associated with one or more atrial-restricted cardiac diseases, optionally wherein the protein is a protein associated with atrial fibrillation.
68. The isolated nucleic acid of paragraph 66 or 67, wherein the protein is a voltage-gated channel protein.
69. The isolated nucleic acid of paragraph 68, wherein the voltage-gated protein is a potassium voltage-gated channel subfamily H member 2 protein (KCNH2 protein).
70. The isolated nucleic acid of paragraph 69, wherein the KCNH2 protein comprises an amino acid sequence that is at least 70%, 80%, 90%, 95%, 97%, 98%, or 99% identical to the amino acid sequence set forth in SEQ ID NO: 19 or 20.
71. The isolated nucleic acid of paragraph 70, wherein the KCNH2 protein comprises or consists of the nucleic acid sequence as set forth in SEQ ID NO: 19 or 20.
72. The isolated nucleic acid of any one of paragraphs 66-71, wherein the nucleic acid sequence encoding a protein is at least 60%, 70%, 80%, 90%, 95% or 99% identical to the sequence set forth in SEQ ID NOs: 1 or 2.
73. The isolated nucleic acid of paragraph 72, wherein the nucleic acid sequence encoding a protein comprises or consists of the nucleic acid sequence as set forth in SEQ ID NO: 1 or 2.
74. The isolated nucleic acid of paragraph 67, wherein the protein associated with one or more atrial-restricted cardiac diseases is Calcium/Calmodulin Dependent Protein Kinase II Inhibitor 2 (CAMK2N2), Sarcoplasmic/Endoplasmic Reticulum Calcium ATPase 2A (SERCA2A), Potassium Two-Pore Domain Channel Subfamily K Member 2 (KCNK2), Connexin 40, or Connexin 43.
75. The isolated nucleic acid of any one of paragraphs 66-74 further comprising adeno-associated virus (AAV) inverted terminal repeats (ITRs).
76. The isolated nucleic acid of paragraph 75, wherein the AAV ITRs are of serotype AAV1, AAV2, AAV6, AAV8, or AAV9.
77. A vector comprising the isolated nucleic acid of any one of paragraphs 66-76.
78 The vector of paragraph 77, wherein the vector is a plasmid or a viral vector.
79. The vector of paragraph 78, wherein the viral vector is an adenoviral vector, adeno-associated viral vector, a lentiviral vector, a retroviral vector, or a Baculovirus vector.
80. A recombinant adeno-associated virus (rAAV) comprising:
(i) the isolated nucleic acid of any one of paragraphs 66-76; and
(ii) an adeno-associated virus (AAV) capsid protein.
81. The rAAV of paragraph 80, wherein the capsid protein has a tropism for heart tissue, optionally wherein the heart tissue is atrial tissue.
82. The rAAV of paragraph 80 or 81, wherein the capsid protein is of a serotype selected from AAV1, AAV2, AAV3, AAV4, AAV5, AAV6, AAV7, AAV8, AAV9 and a variant of any of the foregoing.
83. The rAAV of any one of paragraphs 80-82, wherein the rAAV is formulated for delivery to the heart.
84. A composition comprising the isolated nucleic acid of any one of paragraphs 66-76, the vector of any one of paragraphs 77-79, and/or the rAAV of any one of paragraphs 80-83.
85. The composition of paragraph 84, further comprising a pharmaceutically acceptable excipient.
86. A method for expressing a protein in an atrial cardiomyocyte comprising administering the isolated nucleic acid of any one of paragraphs 66-76 or the composition of paragraph 85 to the atrial cardiomyocyte.
87. The method of paragraph 86, wherein the expression of the protein is increased by at least 10%, 20%, 30%, 50%, 75%, or 100% relative to a control cardiomyocyte.
88. A method for expressing a protein in an atrial cardiomyocyte of a subject comprising administering the isolated nucleic acid of any one of paragraphs 66-76 or the composition of paragraph 85 to the subject.
89. The method of paragraph 88, wherein the expression of the protein is higher in the atrial cardiomyocyte relative to a control cell in the subject, optionally wherein the control cell is a ventricular cardiomyocyte or a liver cell from the same subject.
90. The method of paragraph 88 or 89, wherein the expression of the protein is increased by at least 10%, 20%, 30%, 50%, 75%, or 100% in the atrial cardiomyocyte relative to the control cell.
91. A method of treating an atrial-restricted cardiac disease in a subject in need thereof, comprising administering to the subject the composition of paragraph 85.
92. The method of paragraph 91, wherein the atrial-restricted cardiac disease is atrial fibrillation.
93. The method of any one of paragraphs 88-92, wherein the subject has a cardiac disorder.
94. The method of any one of paragraphs 88-93, wherein the subject has atrial fibrillation.
95. The method of any one of paragraphs 88-94, wherein the subject is human.
96. The method of any one of paragraphs 88-95, wherein the administration is via gene painting.
97. The method of any one of paragraphs 88-95, wherein the administration is intravenous, intramuscular, subcutaneous, or intraarterial administration.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an.” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B.” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of.” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either.” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A truncated potassium voltage-gated channel subfamily H member 2 (KCNH2) protein comprising one or more dominant-negative mutations relative to a wild-type KCNH2 protein and having a length of 500 to 1,150 amino acids.

2. The truncated KCNH2 protein of claim 1, wherein the one or more dominant-negative mutations comprises one or more amino acid substitutions.

3. The truncated KCNH2 protein of claim 2, wherein the one or more amino acid substitutions is at position N470, T473, A561, I593, G626, F627, and/or G628 relative to amino acid position numbering of a wild-type KCNH2 protein (e.g., SEQ ID NO: 19).

4. The truncated KCNH2 protein of claim 3, wherein:
(a) the amino acid substitution at position G628 is G628S, G628A, G628R, G628N, G628D, G628C, G628Q, G628E, G628H, G628I, G628L, G628K, G628M, G628F, G628P, G628S, G628T, G628W, G628Y, or G628V; and/or
(b) the amino acid substitution at position N470 is N470D or N470E; and/or
(c) the amino acid substitution at position T473 is T473P; and/or
(d) the amino acid substitution at position A561 is A561V; and/or
(e) the amino acid substitution at position I593 is I593R or I593K; and/or
(f) the amino acid substitution at position G626 is G626S, G626A, G626R, G626N, G626D, G626C, G626Q, G626E, G626H, G626I, G626L, G626K, G626M, G626F, G626P, G626T, G626W, G626Y, or G626V; and/or
(g) the amino acid substitution at position F627 is F627S, F627A, F627R, F627N, F627D, F627C, F627Q, F627E, F627H, F627I, F627L, F627K, F627M, F627G, F627P, F627T, F627W, F627Y, or F627V.

5. The truncated KCNH2 protein of claim 2, wherein the one or more dominant-negative mutations comprises a deletion, optionally wherein the deletion comprises a deletion of residues 500-508 relative to amino acid position numbering of a wild-type KCNH2 protein.

6. The truncated KCNH2 protein of claim 1, wherein the protein is truncated by at least 10, 20, 30, 50, 100, 150, 200, 250, or more amino acids compared to the amino acid sequence set forth in SEQ ID NO: 19.

7. The truncated KCNH2 protein of claim 1, wherein the protein consists of 500-1,000 amino acids, 500-900 amino acids, 500-800 amino acids, or 650-950 amino acids, optionally 910-930 amino acids.

8. The truncated KCNH2 protein of claim 1 comprising at least 80%, 85%, 90%, 95%, 97%, 98%, or 99% identity to the amino acid sequence of SEQ ID NO: 21.

9. The truncated KCNH2 protein of claim 1, comprising or consisting of the amino acid sequence of SEQ ID NO: 22, 24, 26, 28, 30, 32, 34, or 35.

10. An isolated nucleic acid encoding the truncated KCNH2 protein of claim 1.

11. The isolated nucleic acid of claim 10 comprising a nucleic acid sequence that is at least 70%, 80%, 90%, 95%, 99%, 99.9% identical to the nucleic acid sequence set forth in SEQ ID NOs: 1 or 2.

12. The isolated nucleic acid of claim 10, further comprising:
(i) a Kozak sequence; and/or
(ii) one or more regulatory elements, optionally wherein the one or more regulatory elements comprises a promoter and/or an enhancer, optionally wherein the promoter is an atrial cell-specific promoter, further optionally wherein the atrial cell-specific promoter comprises an Anf1 promoter sequence, optionally wherein the promoter sequence is set forth in SEQ ID NO: 3; and/or
(iii) one or more introns, optionally wherein the one or more introns is positioned between the one or more regulatory element and a nucleic acid sequence encoding the truncated KCNH2 protein; and/or
(iv) a 3′ untranslated region (3′ UTR); and/or
(v) a 5′ untranslated region (5′ UTR); and/or
(vi) adeno-associated virus (AAV) inverted terminal repeats (ITRs) flanking a nucleotide sequence encoding the truncated KCNH2 protein.

13-14. (canceled)

15. A recombinant adeno-associated virus (rAAV) comprising:
(i) an isolated nucleic acid encoding the truncated KCNH2 protein of claim 1; and
(ii) an adeno-associated virus (AAV) capsid protein.

16. The rAAV of claim 15, wherein the capsid protein has a tropism for heart tissue, optionally wherein the heart tissue is atrial tissue, and/or the capsid protein is of a serotype selected from AAV1, AAV2, AAV3, AAV4, AAV5, AAV6, AAV7, AAV8, AAV9, and a variant of any of the foregoing.

17. A composition comprising the truncated KCNH2 protein of claim 1.

18. A method of administering a truncated KCNH2 protein to a subject, the method comprising administering to the subject the truncated KCNH2 protein of claim 1.

19-20. (canceled)

21. A nucleic acid regulatory element comprising (i) an atrial natriuretic peptide (Anf) promoter and (ii) an enhancer element, wherein the enhancer element is not a naturally-occurring Anf enhancer element.

22. The nucleic acid regulatory element of claim 21, wherein:
(a) the naturally-occurring Anf enhancer element consists of the nucleic acid sequence set forth in SEQ ID NO: 6; and/or
(b) the Anf promoter is a mouse Anf promoter or a human Anf promoter; and/or
(c) the enhancer element is a CMV enhancer, optionally wherein the CMV enhancer comprises a nucleic acid sequence that is at least 70%, 80%, 90%, 95%, 97%, 98%, or 99% identical to the nucleic acid sequence set forth in SEQ ID NO: 4.

23-26. (canceled)

27. A method for expressing a protein in an atrial cardiomyocyte comprising administering the isolated nucleic acid of claim 21 to the atrial cardiomyocyte, optionally wherein the atrial cardiomyocyte is in a subject.

28. A method of treating an atrial-restricted cardiac disease in a subject in need thereof, comprising administering to the subject the isolated nucleic acid of claim 21, optionally wherein the atrial-restricted cardiac disease is atrial fibrillation and/or the subject has a cardiac disorder or atrial fibrillation.


SEQUENCE LISTING (35 sequences)

SEQ ID NO: 1 - DNA, 2766 bp, synthetic construct
  source 1..2766: mol_type = other DNA
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggctag

SEQ ID NO: 2 - DNA, 2766 bp, synthetic construct
  source 1..2766: mol_type = other DNA
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttctccaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggctag

SEQ ID NO: 3 - DNA, 974 bp, Mus musculus
  source 1..974: mol_type = genomic DNA
ccccccctcaatcccttacctcctccttctatttccctgggaagggtgggaccaccacatatttcatgctcagagtctgggctttggaaagaagaaaagtggtttcatcctccaaacccatcctgttggcaccttggacacgagtcttgggaggcagtaaaaataaactttagaggaaatatgctcttctaacatcccttggtgtttacacgtgtagctgaattctttagagcctgtatcatgttggcttcctggctgacttcatactctaaaaaaaaaataatagctctttcacctgactgctaacagggacatctagggtgggggtgggctgtctggggccagaggtccacccacgaggccgatgaatcaggtgtgaagctagctccagcatgtgtactccctggccagcctagctggcctcccagctgcctgtcattgcctcctctcccgcccttatttggagcccctgacagctgagcagcaagcagggggagctgggtgcaggccagccgtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataactttaaaagggcatcttctgctggctcctcactccatcgctttatcgctgcaagtgacagaatggggagggttctagcccccctgccttctcaaagagctggggggctataaaaacgggagatgctggcagctaggagacagtgacggacaaaagctgagagagagagagaaagaaaccagagtgggcagagacagcaaacatcagatcgtgccccgacccacgccagc

SEQ ID NO: 4 - DNA, 304 bp, unidentified
  source 1..304: mol_type = genomic DNA; note = Cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 5 - DNA, 1278 bp, synthetic construct
  source 1..1278: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgccccccctcaatcccttacctcctccttctatttccctgggaagggtgggaccaccacatatttcatgctcagagtctgggctttggaaagaagaaaagtggtttcatcctccaaacccatcctgttggcaccttggacacgagtcttgggaggcagtaaaaataaactttagaggaaatatgctcttctaacatcccttggtgtttacacgtgtagctgaattctttagagcctgtatcatgttggcttcctggctgacttcatactctaaaaaaaaaataatagctctttcacctgactgctaacagggacatctagggtgggggtgggctgtctggggccagaggtccacccacgaggccgatgaatcaggtgtgaagctagctccagcatgtgtactccctggccagcctagctggcctcccagctgcctgtcattgcctcctctcccgcccttatttggagcccctgacagctgagcagcaagcagggggagctgggtgcaggccagccgtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataactttaaaagggcatcttctgctggctcctcactccatcgctttatcgctgcaagtgacagaatggggagggttctagcccccctgccttctcaaagagctggggggctataaaaacgggagatgctggcagctaggagacagtgacggacaaaagctgagagagagagagaaagaaaccagagtgggcagagacagcaaacatcagatcgtgccccgacccacgccagc

SEQ ID NO: 6 - DNA, 604 bp, Mus musculus
  source 1..604: mol_type = genomic DNA
ctcagccgttcttcaagtctttgcccaactcaaagaccatctccaggaggtaggtcatgtgacacacacagcaaggagcccgagttcccccagtagggctgctcgcctagtcctctctgctaataataatccccaggcacacaattaagcagtgagcgcatcccagggactcatccggaggcgacagtgtgaacttgcacagatttcagacttcaaggagagattggccaacccaaattggttgcctacaacccccagctttctcaaagggctctctttttctatcttagagaaaacatcaccgcagacacctcttgtgacttccagccacatgacaactccaccaggcagccacagggtcaatgggcgtggaagtcttccaatagcccattaagacggtcagaaagcagagagcagcagaaaatgcacacacttatcacacacacaaagcatacatatgtcgaaaatggctacactggggtgtggagaccacttgttaaaggtggactgcctgctcatgctcctgaggctgccagaagaattcatggctcacttccttggtctcaaaggtcatgaaggacattatggagaatggtgtatgtgt

SEQ ID NO: 7 - DNA, 217 bp, unidentified
  source 1..217: mol_type = genomic DNA; note = Cytomegalovirus
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcatcgctattacc

SEQ ID NO: 8 - DNA, 1010 bp, Homo sapiens
  source 1..1010: mol_type = genomic DNA
cctccttcacccccacctcccttcctccatcggtcaagttgccacgggcacaagcccaggaagggcagggggatgttaacaaacttcagacacgaagagtctgagcatctataaacagcaacggaagaaatgaaattggctgcgtcctctaagcctgtccccgcagcatgctggaggagggtcgcgggggacatggaagaggaggagctttggagagaggatgcttgtgctcccccgccttttcttgctatttctatttgggggttggattcctgggagcttcatcacatttggttctcagctgactttatatactaaaaaataacttcctttcgcctgaccatggagagggactgccaggggtgaaggcagccctgtctgaggccagaggtctgcccacgtggcggatgaggcaggtgtgaggccagcttgagcatctggatccatttgtctcgggctgctggctgcctgccatttcctcctctccacccttatttggaggccctgacagctgagccacaaacaaaccaggggagctgggcaccagccaagcgtcaccctctgtttccccgcacgggtaccagcgtcgaggagaaagaatcctgaggcacggcggtgagataaccaaggactcttttttactcttctcacacctttgaagtgggagcctcttgagtcaaatcagtaagaatgcggctcttgcagctgagggtctggggggctgttggggctgcccaaggcagagaggggctgtgacaagccctgcgggatgataactttaaaagggcatctcctgctggcttctcacttggcagctttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccag

SEQ ID NO: 9 - DNA, 1314 bp, synthetic construct
  source 1..1314: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcctccttcacccccacctcccttcctccatcggtcaagttgccacgggcacaagcccaggaagggcagggggatgttaacaaacttcagacacgaagagtctgagcatctataaacagcaacggaagaaatgaaattggctgcgtcctctaagcctgtccccgcagcatgctggaggagggtcgcgggggacatggaagaggaggagctttggagagaggatgcttgtgctcccccgccttttcttgctatttctatttgggggttggattcctgggagcttcatcacatttggttctcagctgactttatatactaaaaaataacttcctttcgcctgaccatggagagggactgccaggggtgaaggcagccctgtctgaggccagaggtctgcccacgtggcggatgaggcaggtgtgaggccagcttgagcatctggatccatttgtctcgggctgctggctgcctgccatttcctcctctccacccttatttggaggccctgacagctgagccacaaacaaaccaggggagctgggcaccagccaagcgtcaccctctgtttccccgcacgggtaccagcgtcgaggagaaagaatcctgaggcacggcggtgagataaccaaggactcttttttactcttctcacacctttgaagtgggagcctcttgagtcaaatcagtaagaatgcggctcttgcagctgagggtctggggggctgttggggctgcccaaggcagagaggggctgtgacaagccctgcgggatgataactttaaaagggcatctcctgctggcttctcacttggcagctttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccag

SEQ ID NO: 10 - DNA, 23 bp, synthetic construct
  source 1..23: mol_type = other DNA
ctagtaatggggctgcccggtac

SEQ ID NO: 11 - DNA, 19 bp, synthetic construct
  source 1..19: mol_type = other DNA
gatcattaccccgacgggc

SEQ ID NO: 12 - DNA, 3480 bp, Homo sapiens
  source 1..3480: mol_type = genomic DNA
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag

SEQ ID NO: 13 - DNA, 3480 bp, synthetic construct
  source 1..3480: mol_type = other DNA
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttctccaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag

SEQ ID NO: 14 - DNA, 4044 bp, synthetic construct
  source 1..4044: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgccccccctcaatcccttacctcctccttctatttccctgggaagggtgggaccaccacatatttcatgctcagagtctgggctttggaaagaagaaaagtggtttcatcctccaaacccatcctgttggcaccttggacacgagtcttgggaggcagtaaaaataaactttagaggaaatatgctcttctaacatcccttggtgtttacacgtgtagctgaattctttagagcctgtatcatgttggcttcctggctgacttcatactctaaaaaaaaaataatagctctttcacctgactgctaacagggacatctagggtgggggtgggctgtctggggccagaggtccacccacgaggccgatgaatcaggtgtgaagctagctccagcatgtgtactccctggccagcctagctggcctcccagctgcctgtcattgcctcctctcccgcccttatttggagcccctgacagctgagcagcaagcagggggagctgggtgcaggccagccgtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataactttaaaagggcatcttctgctggctcctcactccatcgctttatcgctgcaagtgacagaatggggagggttctagcccccctgccttctcaaagagctggggggctataaaaacgggagatgctggcagctaggagacagtgacggacaaaagctgagagagagagagaaagaaaccagagtgggcagagacagcaaacatcagatcgtgccccgacccacgccagcatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttctccaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggctag

SEQ ID NO: 15 - DNA, 4080 bp, synthetic construct
  source 1..4080: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcctccttcacccccacctcccttcctccatcggtcaagttgccacgggcacaagcccaggaagggcagggggatgttaacaaacttcagacacgaagagtctgagcatctataaacagcaacggaagaaatgaaattggctgcgtcctctaagcctgtccccgcagcatgctggaggagggtcgcgggggacatggaagaggaggagctttggagagaggatgcttgtgctcccccgccttttcttgctatttctatttgggggttggattcctgggagcttcatcacatttggttctcagctgactttatatactaaaaaataacttcctttcgcctgaccatggagagggactgccaggggtgaaggcagccctgtctgaggccagaggtctgcccacgtggcggatgaggcaggtgtgaggccagcttgagcatctggatccatttgtctcgggctgctggctgcctgccatttcctcctctccacccttatttggaggccctgacagctgagccacaaacaaaccaggggagctgggcaccagccaagcgtcaccctctgtttccccgcacgggtaccagcgtcgaggagaaagaatcctgaggcacggcggtgagataaccaaggactcttttttactcttctcacacctttgaagtgggagcctcttgagtcaaatcagtaagaatgcggctcttgcagctgagggtctggggggctgttggggctgcccaaggcagagaggggctgtgacaagccctgcgggatgataactttaaaagggcatctcctgctggcttctcacttggcagctttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccagatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttctccaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggctag

SEQ ID NO: 16 - DNA, 821 bp, synthetic construct
  source 1..821: mol_type = other DNA
ctcagccgttcttcaagtctttgcccaactcaaagaccatctccaggaggtaggtcatgtgacacacacagcaaggagcccgagttcccccagtagggctgctcgcctagtcctctctgctaataataatccccaggcacacaattaagcagtgagcgcatcccagggactcatccggaggcgacagtgtgaacttgcacagatttcagacttcaaggagagattggccaacccaaattggttgcctacaacccccagctttctcaaagggctctctttttctatcttagagaaaacatcaccgcagacacctcttgtgacttccagccacatgacaactccaccaggcagccacagggtcaatgggcgtggaagtcttccaatagcccattaagacggtcagaaagcagagagcagcagaaaatgcacacacttatcacacacacaaagcatacatatgtcgaaaatggctacactggggtgtggagaccacttgttaaaggtggactgcctgctcatgctcctgaggctgccagaagaattcatggctcacttccttggtctcaaaggtcatgaaggacattatggagaatggtgtatgtgtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcatcgctattacc

SEQ ID NO: 17 - DNA, 1578 bp, Mus musculus
  source 1..1578: mol_type = genomic DNA
ctcagccgttcttcaagtctttgcccaactcaaagaccatctccaggaggtaggtcatgtgacacacacagcaaggagcccgagttcccccagtagggctgctcgcctagtcctctctgctaataataatccccaggcacacaattaagcagtgagcgcatcccagggactcatccggaggcgacagtgtgaacttgcacagatttcagacttcaaggagagattggccaacccaaattggttgcctacaacccccagctttctcaaagggctctctttttctatcttagagaaaacatcaccgcagacacctcttgtgacttccagccacatgacaactccaccaggcagccacagggtcaatgggcgtggaagtcttccaatagcccattaagacggtcagaaagcagagagcagcagaaaatgcacacacttatcacacacacaaagcatacatatgtcgaaaatggctacactggggtgtggagaccacttgttaaaggtggactgcctgctcatgctcctgaggctgccagaagaattcatggctcacttccttggtctcaaaggtcatgaaggacattatggagaatggtgtatgtgtccccccctcaatcccttacctcctccttctatttccctgggaagggtgggaccaccacatatttcatgctcagagtctgggctttggaaagaagaaaagtggtttcatcctccaaacccatcctgttggcaccttggacacgagtcttgggaggcagtaaaaataaactttagaggaaatatgctcttctaacatcccttggtgtttacacgtgtagctgaattctttagagcctgtatcatgttggcttcctggctgacttcatactctaaaaaaaaaataatagctctttcacctgactgctaacagggacatctagggtgggggtgggctgtctggggccagaggtccacccacgaggccgatgaatcaggtgtgaagctagctccagcatgtgtactccctggccagcctagctggcctcccagctgcctgtcattgcctcctctcccgcccttatttggagcccctgacagctgagcagcaagcagggggagctgggtgcaggccagccgtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataactttaaaagggcatcttctgctggctcctcactccatcgctttatcgctgcaagtgacagaatggggagggttctagcccccctgccttctcaaagagctggggggctataaaaacgggagatgctggcagctaggagacagtgacggacaaaagctgagagagagagagaaagaaaccagagtgggcagagacagcaaacatcagatcgtgccccgacccacgccagc

SEQ ID NO: 18 - DNA, 521 bp, unidentified
  source 1..521: mol_type = genomic DNA; note = Cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcatcgctattacc

SEQ ID NO: 19 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 20 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFSNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 21 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 22 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFSNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 23 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILIDFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 24 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILIDFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 25 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILIEFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 26 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILIEFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 27 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRPTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 28 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRPTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 29 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIVHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 30 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIVHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 31 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQRGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 32 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQRGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 33 - protein, 1159 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQKGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 34 - protein, 921 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQKGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG

SEQ ID NO: 35 - protein, 912 aa, synthetic construct
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRG